(19) United States
(12) Patent Application Publication
(10) Pub. No.: US 2022/0251202 A1
(43) Pub. Date: Aug. 11, 2022

(54) FUSIONS OF MUTANT INTERLEUKIN-2 POLYPEPTIDES WITH ANTIGEN BINDING MOLECULES FOR MODULATING IMMUNE CELL FUNCTION

(71) Applicant: Asher Biotherapeutics, Inc., South San Francisco, CA (US)

(72) Inventors: Ivana DJURETIC, San Bruno, CA (US); Yik Andy YEUNG, South San Francisco, CA (US)

(21) Appl. No.: 17/616,157

(22) PCT Filed: Jun. 5, 2020
(86) PCT No.: PCT/US20/36454

§ 371 Date: Dec. 2, 2021

Related U.S. Application Data
(60) Provisional application No. 62/857,726, filed on Jun. 5, 2019.

(51) Int. Cl. C07K 16/28; C07K 14/55; A61P 35/00

(52) U.S. Cl. CPC C07K 16/2815; C07K 14/55; A61P 35/00; C07K 2317/51; C07K 2317/515; A61K 2039/505; C07K 2317/52; C07K 2319/30; C07K 2317/73; C07K 2317/92; C07K 2317/56

(57) ABSTRACT

Provided herein are fusion proteins that bind human CD8α, human CD8β, or human PD1 and comprise a mutant IL-2 polypeptide, as well as polynucleotides, host cells, compositions, and methods of use thereof.

### CROSS REFERENCE TO RELATED APPLICATIONS

This application claims priority to U.S. Provisional Application No. 62/857,726, filed Jun. 5, 2019, the disclosures of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 182842000140SEQLIST.TXT, date recorded: Jun. 5, 2020, size: 105 KB).

### FIELD

The present disclosure discloses mutant interleukin-2 polypeptides, and fusion polypeptides comprising the mutant interleukin-2 polypeptides and antigen binding molecules. The present disclosure provides methods of modulating immune cell function by contacting the immune cell with fusion polypeptides of the present disclosure. In addition, the present disclosure also provides polynucleotides encoding the disclosed fusion proteins, and vectors and host cells comprising such polynucleotides. The present disclosure further provides methods for producing the fusion proteins, pharmaceutical compositions comprising the same, and uses thereof.

### BACKGROUND

Interleukin-2 (IL-2) is a cytokine that regulates many lymphocyte subsets, including alpha beta CD4+ and CD8 T+ cells, and various innate and innate-like lymphocytes such as NK cells, NK T cells, gamma delta T cells (Tγδ) cells, and innate lymphoid cells (ILC1, ILC2, and ILC3 cells). Binding of IL-2 to its receptor induces the phosphorylation of receptor-associated Janus kinases, JAK3 and JAK1, which promote the phosphorylation of STAT5 transcription factor (pSTAT5) that regulates transcription of many genes in lymphocytes. In addition to STAT5, binding of IL-2 to its receptor also activates other signaling pathways such as ERK, PI3K, and Akt kinases. IL-2 signaling in lymphocytes promotes cell survival, proliferation, and increased effector function, including pro-inflammatory cytokine secretion and cytotoxic function, and in some cases, activation-induced cell death (reviewed in Ross & Cantrell, Annu Rev Immunol. Apr. 26, 2018; 36:411-433).

IL-2 can signal by binding with an intermediate affinity to a receptor complex consisting of IL-2Rβ and IL-2Rγ subunits (IL-2Rβγ, intermediate affinity receptor), both of which are required and sufficient to trigger downstream signaling in immune cells. In addition, IL-2 binds with high affinity to a receptor complex consisting of IL-2Rα, IL-2Rβ, and IL-2Rγ subunits (IL-2Rαβγ, high affinity receptor) (Stauber et al, Proc Natl Acad Sci USA. Feb. 21, 2006; 103(8):2788-93). IL-2Rα expression is restricted to CD4+ Treg cells, activated T lymphocytes, and ILC2 and ILC3 cells, making these subsets the most sensitive to IL-2 signaling. IL-2Rβ and IL-2Rγ subunits are shared with another related cytokine, IL-15, and IL-2Rγ subunit is shared among other common gamma chain cytokines (IL-4, IL-7, IL-9, and IL-21). Most innate and innate-like lymphocytes including NK cells, NK T cells, Tγδ cells, and ILC1, ILC2, and ILC3 cells express high levels of IL-2Rβ (ImmGen consortium; Heng T S et al, Immunological Genome Project Consortium. Nat Immunol. 2008 October; 9(10):1091-4), which also makes them sensitive to both IL-2 and IL-15 cytokines.

In agreement with its potent activities on lymphocytes, systemic administration of high dose of IL-2 resulted in activation of anti-tumor immune responses and efficacy in many preclinical cancer models. Systemically administered high dose IL-2 has also been tested in patients and high dose IL-2 is approved for the treatment of metastatic melanoma and renal cell carcinoma (RCC). Dosing regimen consisted of intravenous injections of 600,000 IU/kg every 8 hr, established based on the in vivo half-life of IL-2 in order to maintain serum levels at concentrations necessary to stimulate high-affinity IL-2 receptors. Overall response rate in RCC was 20% with complete response rate of 9%, while overall response rate in melanoma was 16% with complete response rate of 6% (reviewed in Rosenberg, J Immunol. Jun. 15, 2014; 192(12):5451-8). The efficacy of high dose IL-2 in cancer is attributed to its ability to potently expand T cells and NK cells while maintaining their function. However, IL-2 also expands Treg cells and promotes their proper suppressive function (Chinen et al, Nat Immunol. 2016 November; 17(11):1322-1333). In fact, due to the sensitivity of Tregs to IL-2, low dose IL-2 therapeutic regimens have been tested in patients with autoimmunity to suppress the pathogenic immune responses (Collison, Nat Rev Rheumatol. 2019 January; 15(1):2).

In addition to its undesired effects on immune-suppressive Treg cells, the benefits of IL-2 in patients are accompanied by severe toxicity, including fever, chills, malaise, arthralgias, hypotension abnormal liver function, renal failure, and capillary leak syndrome and fluid retention. IL-2 induced-toxicities set the limitation on the number of doses that patients could receive, and IL-2 treatment requires strict patient-eligibility criteria and administration by experienced physicians (Schwartz et al, Oncology (Williston Park). 2002 November; 16 (11 Suppl 13):11-20). The toxicity of IL-2 involves a complex set of interactions between the immune cells and the vascular endothelium: IL-2-activated cells strongly bind to endothelial cells leading to their lysis, and IL-2 induces pulmonary edema via its interaction with functional IL-2 receptors on endothelial cells (reviewed in Milling et al, Adv Drug Deliv Rev. May 15, 2017; 114: 79-101). Blocking of the IL-2 interaction with IL-2Rα abrogated pulmonary edema in animal models (Krieg et al, Proc Natl Acad Sci USA. Jun. 29, 2010; 107(26):11906-11). In addition, the same study showed that blockade of IL-2Rα also led to vigorous activation of IL-2Rβγ+ effector immune cells, CD8+ T cells and NK cells, and to a lesser extent, also Tregs, substantially improving both safety and anti-tumor efficacy compared to recombinant IL-2.

Recently, NK cells have been shown to cause toxicity of IL-2 in mice through their hyper-activation and secretion of multiple inflammatory cytokines when IL-2 was administered together with IFN-α (Rothschilds et al, Oncoimmunology. Feb. 19, 2019; 8 (5): e1558678). In addition, NK cells were also shown to cause toxicity of the cytokine IL-15 that also signals through IL-2Rβγ (Guo et al, J Immunol. Sep. 1, 2015; 195(5):2353-64). This NK cell hyper-activation in response to IL-2Rβγ signaling is likely due to their high expression of IL-2Rβ and ability to rapidly secrete inflammatory cytokines in response to activation. In addition, although their role in inducing toxicity of IL-2 has not been studied, other innate lymphocytes that also express high levels of IL-2Rβ may play a role in systemic toxicities observed with systemic administration of IL-2.

On the other hand, CD8+ T cells have been shown to mediate efficacy of immunotherapeutic agents, including IL-2, in many preclinical cancer models (Caudana et al, Cancer Immunol Res. 2019 March; 7(3):443-457), and they have also been correlated with response to immunotherapies in patients (Sade-Feldman et al, Cell. Nov. 1, 2018; 175(4):998-1013). CD8+ T cells express CD8, which is a type I transmembrane glycoprotein found on the cell surface as a CD8 alpha (CD8α, CD8a) homodimer and CD8 alpha-CD8 beta (CD8β, CD8b) heterodimer. CD8 dimers interact with the major histocompatibility (MEC) class I molecules on target cells and this interaction keeps the TCR closely engaged with MEC during CD8⁺ T cell activation. The cytoplasmic tail of CD8α contains binding sites for a T cell kinase (Lck) that initiates signal transduction downstream of the TCR during T cell activation, while the role of CD8β is thought to be in increasing the avidity of CD8 binding to MEC class I and influencing specificity of the CD8/MHC/TCR interaction (Bosselut et al, Immunity. 2000 April; 12(4):409-18).

Intratumoral T cells were recently shown to express PD1 in multiple human cancers (Gros et al, J Clin Invest. 2014 May; 124(5):2246-59; Egelston et al, Nat Commun. Oct. 16, 2018; 9(1):4297; Thommen et al, Nat Med. 2018 July; 24(7):994-1004). PD1 is a type I transmembrane protein that contains an extracellular domain, a transmembrane region and a cytoplasmic tail. The cytoplasmic tail contains phosphorylation sites that are part of an immunoreceptor tyrosine-based inhibitory motif (ITIM) that can recruit intracellular phosphatases such as SHP-1 and SHP-2. PD1 negatively regulates TCR signaling by binding to its ligands PD-L1 and PD-L2. The interaction between PD1 and its ligands is blocked by several approved anti-PD1 and anti-PD-L1 antibodies as a treatment for cancer (Ribas & Wolchok, Science. Mar. 23, 2018; 359(6382):1350-1355).

High expression of PD1 on intratumoral T cells is associated with specificity for tumor antigens, and the frequency of these PD1+ T cells in tumors was associate with response to anti-PD1 antibodies (Thommen et al, Nat Med. 2018 July; 24(7):994-1004). PD1 is also expressed on peripheral blood CD8+ and CD4+ memory and effector T cells, albeit at a lower level than on tumor antigen-specific intratumoral T cells, and it can also be expressed on T cells residing in healthy tissues. In addition, other cell types such as Tregs, Tγδ, NK T and ILC2 cells can also express PD1.

The goal was to reduce the toxicity of IL-2 and improve its efficacy by enhancing its activity on CD8+ T cells or PD1+ T cells that have been associated with efficacy in preclinical cancer models and cancer patients and reducing its activity on other cells that have been associated with toxicity and undesired effects of IL-2, including Tregs and innate lymphoid cells.

### BRIEF SUMMARY

The present disclosure describes, inter alia, mutant IL-2 polypeptides, wherein the mutant IL-2 polypeptides have one, two or more, or three or more amino acid substitutions (i.e. mutations) relative to the wild-type mature IL-2 amino acid sequence, e.g., as depicted in FIG. 1A (SEQ ID NO:1) and FIG. 2. In some embodiments, the mutant IL-2 polypeptides exhibit reduced binding affinity to IL-2Rα polypeptide having an amino acid sequence depicted in FIG. 1B (SEQ ID NO:2), compared to the binding affinity of the wild-type IL-2 polypeptide. In some embodiments, the mutant IL-2 polypeptides exhibit reduced binding affinity to IL-2Rα polypeptide having an amino acid sequence depicted in FIG. 1B (SEQ ID NO:2), compared to the binding affinity of the wild-type IL-2 polypeptide; and exhibit reduced binding affinity to IL-2Rβ polypeptide having an amino acid sequence depicted in FIG. 1C (SEQ ID NO:3), compared to the binding affinity of the wild-type IL-2 polypeptide. In some embodiments, the mutant IL-2 polypeptides exhibit reduced binding affinity to IL-2Rα polypeptide having an amino acid sequence depicted in FIG. 1B (SEQ ID NO:2), compared to the binding affinity of the wild-type IL-2 polypeptide; and exhibit reduced binding affinity to IL-2Rγ polypeptide having an amino acid sequence depicted in FIG. 1D (SEQ ID NO:4) compared to the binding affinity of the wild-type IL-2 polypeptide. In some embodiments, the mutant IL-2 polypeptides exhibit reduced binding affinity to IL-2Rα polypeptide having an amino acid sequence depicted in FIG. 1B (SEQ ID NO:2), compared to the binding affinity of the wild-type IL-2 polypeptide; exhibit reduced binding affinity to IL-2Rβ polypeptide having an amino acid sequence depicted in FIG. 1C (SEQ ID NO:3), compared to the binding affinity of the wild-type IL-2 polypeptide; and exhibit reduced binding affinity to IL-2Rγ polypeptide having an amino acid sequence depicted in FIG. 1D (SEQ ID NO:4) compared to the binding affinity of the wild-type IL-2 polypeptide. In some embodiments, mutant IL-2 polypeptides exhibit improved biophysical properties compared to the wild-type IL-2 polypeptide.

Mutant IL-2 polypeptides disclosed herein, due to their decreased binding affinity for IL-2R complex, have decreased ability, compared to wild-type IL-2, to bind to and/or stimulate immune cells associated with undesired effects of IL-2 on efficacy, such as Tregs, or with toxicity of IL-2, such as innate lymphoid cells, including NK cells. However, disclosed mutant IL-2 polypeptides also have decreased ability, compared to wild-type IL-2, to bind to and/or activate desired IL-2R-expressing immune cells, such as CD8+ T cells, that have been associated with efficacy in preclinical cancer models and response to immune therapy in patients. In order to turn disclosed mutant IL-2 polypeptides into therapeutics that could be both safer and more effective for the treatment of cancer and other immune-related diseases such as certain infectious diseases, we designed fusion proteins comprising of disclosed mutant IL-2 polypeptides and antigen binding molecules, such as antibodies, for antigens present on CD8+ T cells, such as CD8 and PD1. Such fusion proteins comprising mutant IL-2 polypeptides and antibodies binding specific antigens are also referred to as “targeted” fusion proteins as they bind to antigens recognized by the antigen binding molecules of the fusion. This distinguishes them from “untargeted” fusion proteins comprising mutant IL-2 polypeptides and control antibodies that do not bind to any particular antigens (i.e. Fc fusions or control antibody fusions with IL-2 polypeptides; Zhu et al, Cancer Cell. Apr. 13, 2015; 27(4):489-501).

Without wishing to be bound to theory, FIG. 3 depicts the general mechanism for how antigen binding molecules binding to an antigen on CD8+ T cells could work to increase the binding and/or stimulation of CD8+ T cells by the mutant IL-2 polypeptides in the context of the disclosed targeted fusion proteins containing said mutant IL-2 polypeptides. Certain antigen binding molecules, when fused to mutant IL-2 polypeptides, have the ability to substantially increase the binding and/or activity of the mutant IL-2 polypeptides only on cells expressing the antigen for the antigen binding molecule of the fusion, resulting in preferential activation of antigen-expressing over antigen-non expressing cells (FIG. 3). Unlike targeted fusion proteins, untargeted fusion proteins containing the same mutant IL-2 polypeptide, do not preferentially bind to and/or activate antigen-expressing cells (FIG. 3).

Without wishing to be bound to theory, it is thought that the difference in activation of antigen-expressing over antigen-non expressing cells by the targeted fusion protein, and the difference in activation of antigen-expressing cells by the targeted and the untargeted fusion protein are important for the effectiveness of the targeted fusion protein as a therapeutic and can be measured experimentally. The more selective a fusion protein is for cells that associate with efficacy, such as CD8+ T cells, over other cells that associate with toxicity or undesired effects on efficacy, the greater its therapeutic index may be when the fusion protein is used as a therapeutic.

Certain aspects of the present disclosure relate to fusion proteins comprising two moieties. In some embodiments, the first moiety comprises an antibody heavy chain VH—CH1-hinge-CH2-CH3 monomer wherein VH is a variable heavy chain and CH2-CH3 is a Fc domain, an antibody light chain VL-CL wherein VL is a variable light chain and CL is a constant light chain, and a mutant IL-2 polypeptide, wherein the N-terminus of the mutant IL-2 polypeptide is fused to the C-terminus of the Fc domain via a linker; and the second moiety comprises an antibody heavy chain VH—CH1-hinge-CH2-CH3 monomer and an antibody light chain VL-CL; wherein, both the first and second moiety bind to an epitope on one antigen selected from the following group: human CD8α, human CD8β, and human PD1. In some embodiments, the first moiety is a polypeptide comprising an antibody hinge-CH2-CH3 monomer wherein CH2-CH3 is a Fc domain, and a mutant IL-2 polypeptide, wherein the N-terminus of the mutant IL-2 polypeptide is fused to the C-terminus end of the Fc domain via a linker; and 5he second moiety is a polypeptide comprising an antibody heavy chain VH—CH1-hinge-CH2-CH3 monomer and an antibody light chain VL-CL; wherein the second moiety binds to an epitope on one antigen selected from the following group: human CD8α, human CD8β, and human PD1. In some embodiments, the first moiety is a polypeptide comprising an antibody hinge-CH2-CH3 monomer wherein CH2-CH3 is a Fc domain, and a mutant IL-2 polypeptide, wherein the C-terminus of the mutant IL-2 polypeptide is fused to the N-terminus end of the Fc domain via a linker; and the second moiety is a polypeptide comprising an antibody heavy chain VH—CH1-hinge-CH2-CH3 monomer and an antibody light chain VL-CL; wherein the second moiety binds to an epitope on one antigen selected from the following group: human CD8α, human CD8β, and human PD1. In some embodiments, the first moiety comprises an antigen-binding domain that binds to human CD8α or human CD8β; and the second moiety comprises a mutant IL-2 polypeptide; wherein the second moiety is linked to the first moiety via a linker (e.g., the second moiety is fused to the first moiety).

In some embodiments, said mutant IL-2 polypeptide exhibits reduced binding affinity by 50% or more to IL-2Rα polypeptide having an amino acid sequence of SEQ ID NO:2, compared to the binding affinity of the wild-type IL-2 polypeptide with an amino acid sequence of SEQ ID NO:1. In some embodiments, said mutant IL-2 polypeptide exhibits reduced binding affinity by 50% or more to IL-2Rα polypeptide having an amino acid sequence of SEQ ID NO:2, compared to the binding affinity of the wild-type IL-2 polypeptide with an amino acid sequence of SEQ ID NO:1, and reduced binding affinity by 50% or more to IL-2Rβ polypeptide having an amino acid sequence of SEQ ID NO:3, compared to the binding affinity of the wild-type IL-2 polypeptide with an amino acid sequence of SEQ ID NO:1. In some embodiments, said mutant IL-2 polypeptide exhibits reduced binding affinity by 50% or more to IL-2Rα polypeptide having an amino acid sequence of SEQ ID NO:2, compared to the binding affinity of the wild-type IL-2 polypeptide with an amino acid sequence of SEQ ID NO:1, and reduced binding affinity by 50% or more to IL-2Rγ polypeptide having an amino acid sequence of SEQ ID NO:4, compared to the binding affinity of the wild-type IL-2 polypeptide with an amino acid sequence of SEQ ID NO:1. In some embodiments, said mutant IL-2 polypeptide exhibits reduced binding affinity by 50% or more to IL-2Rα polypeptide having an amino acid sequence of SEQ ID NO:2, compared to the binding affinity of the wild-type IL-2 polypeptide with an amino acid sequence of SEQ ID NO:1, and reduced binding affinity by 50% or more to IL-2Rβ polypeptide having an amino acid sequence of SEQ ID NO:3, compared to the binding affinity of the wild-type IL-2 polypeptide with an amino acid sequence of SEQ ID NO:1, and reduced binding affinity by 50% or more to IL-2Rγ polypeptide having an amino acid sequence of SEQ ID NO:4, compared to the binding affinity of the wild-type IL-2 polypeptide with an amino acid sequence of SEQ ID NO:1.

In some embodiments, binding affinity of a mutant IL-2 polypeptide to IL-2Rα is measured by comparing activation of a Treg cell by a fusion protein of the present disclosure (e.g., comprising an anti-CD8 antigen-binding domain of the present disclosure and the mutant IL-2 polypeptide), as compared to activation of a Treg cell by wild-type IL-2, and by comparing activation of a NK cell (expressing IL-2Rbg) by a fusion protein of the present disclosure (e.g., comprising an anti-CD8 antigen-binding domain of the present disclosure and the mutant IL-2 polypeptide), as compared to activation of a NK cell by wild-type IL-2 or an IL-2 polypeptide with no binding to IL-2Rα and wild-type like binding to IL-2Rb and IL2Rg.

In some embodiments, binding affinity to IL-2Rβ or IL-2Rγ of a mutant IL-2 polypeptide with reduced or no binding affinity to IL-2Rα is measured by comparing activation of a cell expressing IL-2Rβ and IL-2Rγ by a fusion protein of the present disclosure (e.g., comprising an anti-CD8 antigen-binding domain of the present disclosure and the mutant IL-2 polypeptide), as compared to activation of a cell expressing IL-2Rβ or IL-2Rγ by wild-type IL-2 or an IL-2 polypeptide with no binding to IL-2Rα and wild-type like binding to IL-2Rb and IL2Rg. For example, a mutant IL-2 polypeptide with reduced or no binding to IL-2Rα(or a fusion protein comprising the same) can be optionally further mutated and tested for activation of cells that express IL-2Rα/β/γ, e.g., Treg cells or IL-2bg, i.e. NK cells. Since the mutant IL-2 polypeptide has no binding to IL-2Rα, the ability to activate, or the potency of activation of, cells that express IL-2Rα/β/γ can be used as an assay for binding of the mutant IL-2 polypeptide or fusion protein to IL-2Rβ/γ.

In some embodiments, the fusion protein activates CD8+ T cells with 10-fold or greater potency, or 50-fold or greater potency, as compared to activation of NK cells. In some embodiments, said mutant IL-2 polypeptide comprises the sequence of SEQ ID NO:1 with one or more or two or more amino acid substitutions relative to SEQ ID NO:1, and wherein the substitutions are at positions of SEQ ID NO:1 selected from the group consisting of: 011, H16, L18, L19, D20, Q22, R38, F42, K43, Y45, E62, P65, E68, V69, L72, D84, S87, N88, V91, 192, T123, 0126, S127, I129, and 5130. In some embodiments, said mutant IL-2 polypeptide comprises an F42A or F42K amino acid substitution relative to SEQ ID NO:1. In some embodiments, said mutant IL-2 polypeptide further comprises an R38A, R38D, R38E, E62Q, E68A, E68Q, E68K, or E68R amino acid substitution relative to SEQ ID NO:1. In some embodiments, said mutant IL-2 polypeptide further comprises an H16E, H16D, D20N, M23A, M23R, M23K, S87K, S87A, D84L, D84N, D84V, D84H, D84Y, D84R, D84K, N88A, N88S, N88T, N88R, N88I, V91A, V91T, V91E, I92A, E95S, E95A, E95R, T123A, T123E, T123K, T123Q, Q126A, Q126S, Q126T, Q126E, S127A, S127E, S127K, or S127Q amino acid substitution relative to SEQ ID NO:1. In some embodiments, said mutant IL-2 polypeptide comprises an amino acid sequence selected from the group consisting of SEQ ID Nos:18-88. In some embodiments, said mutant IL-2 polypeptide comprises the amino acid sequence of SEQ ID NO:1 with one of the following sets of amino acid substitutions (relative to the sequence of SEQ ID NO:1): R38E and F42A; R38D and F42A; F42A and E62Q; R38A and F42K; R38E, F42A, and N88S; R38E, F42A, and N88A; R38E, F42A, and V91E; R38E, F42A, and D84H; H16D, R38E and F42A; H16E, R38E and F42A; R38E, F42A and Q126S; R38D, F42A and N88S; R38D, F42A and N88A; R38D, F42A and V91E; R38D, F42A, and D84H; H16D, R38D and F42A; H16E, R38D and F42A; R38D, F42A and Q126S; R38A, F42K, and N88S; R38A, F42K, and N88A; R38A, F42K, and V91E; R38A, F42K, and D84H; H16D, R38A, and F42K; H16E, R38A, and F42K; R38A, F42K, and Q126S; F42A, E62Q, and N88S; F42A, E62Q, and N88A; F42A, E62Q, and V91E; F42A, E62Q, and D84H; H16D, F42A, and E62Q; H16E, F42A, and E62Q; F42A, E62Q, and Q126S; R38E, F42A, and C125A; R38D, F42A, and C125A; F42A, E62Q, and C125A; R38A, F42K, and C125A; R38E, F42A, N88S, and C125A; R38E, F42A, N88A, and C125A; R38E, F42A, V91E, and C125A; R38E, F42A, D84H, and C125A; H16D, R38E, F42A, and C125A; H16E, R38E, F42A, and C125A; R38E, F42A, C125A and Q126S; R38D, F42A, N88S, and C125A; R38D, F42A, N88A, and C125A; R38D, F42A, V91E, and C125A; R38D, F42A, D84H, and C125A; H16D, R38D, F42A, and C125A; H16E, R38D, F42A, and C125A; R38D, F42A, C125A, and Q126S; R38A, F42K, N88S, and C125A; R38A, F42K, N88A, and C125A; R38A, F42K, V91E, and C125A; R38A, F42K, D84H, and C125A; H16D, R38A, F42K, and C125A; H16E, R38A, F42K, and C125A; R38A, F42K, C125A and Q126S; F42A, E62Q, N88S, and C125A; F42A, E62Q, N88A, and C125A; F42A, E62Q, V91E, and C125A; F42A, E62Q, and D84H, and C125A; H16D, F42A, and E62Q, and C125A; H16E, F42A, E62Q, and C125A; and F42A, E62Q, C125A and Q126S. In some embodiments, said mutant IL-2 polypeptide comprises the amino acid sequence of any of: IL-2 m1, IL-2m2, IL-2m3, IL-2m4, IL-2m4.9, IL-2m4.10, IL-2m4.11, IL-2m4.12, IL-2m4.13, IL-2m4.14, IL-2m4.15, IL-2m4.16, IL-2m4.17, IL-2m4.2, IL-2m4.1, IL-2m4.6, IL-2m4.18, IL-2m4.4, IL-2m4.19, IL-2m4.5, IL-2m4.20, IL-2m4.3, IL-2m4.21, IL-2m4.22, IL-2m4.23, IL-2m4.24, IL-2m5, IL-2m6, IL-2m7, IL-2m8, IL-2m9, IL-2m10, IL-2m10.1, IL-2m10.2, IL-2m10.3, IL-2m10.4, IL-2m10.5, IL-2m10.6, IL-2m10.7, IL-2m10.8, IL-2m10.9, IL-2m10.10, IL-2m10.11, as described herein. In some embodiments, the fusion protein binds human CD8, and binding of the fusion protein to CD8 does not block the interaction of CD8 with MHC class I. In some embodiments, said mutant IL-2 polypeptide further comprises the amino acid mutation C125A compared to SEQ ID NO: 1. In some embodiments, said first and second Fc domains comprise the following Fc mutations according to EU numbering: L234A, L235A, G237A, and K322A. In some embodiments, said first Fc domain comprises the following amino acid substitutions: Y349C and T366W, and wherein said second Fc domain comprises the following amino acid substitutions: S354C, T366S, L368A and Y407V, according to EU numbering; or said second Fc domain comprises the following amino acid substitutions: Y349C and T366W, and wherein said first Fc domain comprises the following amino acid substitutions: S354C, T366S, L368A and Y407V, according to EU numbering. In some embodiments, the fusion protein has or exhibits one or more of the following properties: the fusion protein binds human CD8, and the binding of the fusion protein to CD8 does not block the interaction of CD8 with MHC class I; and the fusion protein activates CD8+ T cells with 10-fold or greater potency, as compared to activation of NK cells. In some embodiments, potency of activation of CD8+ T cells and NK cells is measured by EC50 of cell activation, as assessed by cell proliferation (e.g., a Ki67 assay). In some embodiments, potency of activation of CD8+ T cells and NK cells is measured by EC50 of cell activation, as assessed by STAT5 activation (e.g., pSTAT5 assay).

Other aspects of the present disclosure relate to isolated polynucleotide(s) (e.g., one or more) encoding the mutant IL-2 polypeptides or fusion protein according to any one of the above embodiments. Other aspects of the present disclosure relate to vector(s) (e.g., one or more) encoding the mutant IL-2 polypeptides, fusion protein, or isolated polynucleotide(s) according to any one of the above embodiments. In some embodiments, the vector(s) (e.g., one or more) are expression vector(s). Other aspects of the present disclosure relate to host cells (e.g., isolated and/or recombinant host cells) comprising the polynucleotide(s) and/or vector(s) according to any one of the above embodiments. Other aspects of the present disclosure relate to pharmaceutical compositions comprising the fusion protein according to any one of the above embodiments and a pharmaceutically acceptable carrier. Other aspects of the present disclosure relate to uses of the fusion proteins or pharmaceutical compositions according to any one of the above embodiments as a medicament. Other aspects of the present disclosure relate to uses of the fusion proteins or pharmaceutical compositions according to any one of the above embodiments in the manufacture of a medicament. Other aspects of the present disclosure relate to uses of the fusion proteins or pharmaceutical compositions according to any one of the above embodiments in a method of treating cancer or chronic infection, wherein said method comprises administering to a patient in need thereof an effective amount of the fusion protein or pharmaceutical composition. Other aspects of the present disclosure relate to uses of the fusion proteins or pharmaceutical compositions according to any one of the above embodiments in a method of treating cancer, wherein said method comprises administering to a patient in need thereof an effective amount of the fusion protein or pharmaceutical composition in combination with a T cell therapy, cancer vaccine, chemotherapeutic agent, or immune checkpoint inhibitor (ICI). Other aspects of the present disclosure relate to uses of the fusion proteins or pharmaceutical compositions according to any one of the above embodiments in the manufacture of a medicament for treating cancer or chronic infection. Other aspects of the present disclosure relate to methods of treating cancer or chronic infection, comprising administering to a patient in need thereof an effective amount of the fusion protein or pharmaceutical composition according to any one of the above embodiments. Other aspects of the present disclosure relate to methods of treating cancer, comprising administering to a patient in need thereof an effective amount of the fusion protein or pharmaceutical composition according to any one of the above embodiments in combination with a T cell therapy, cancer vaccine, chemotherapeutic agent, or immune checkpoint inhibitor (ICI). In some embodiments according to any of the embodiments described herein, the ICI is an inhibitor of PD-1, PD-L1, or CTLA-4.

In some embodiments, disclosed targeted IL-2 fusion proteins containing antigen binding molecules activate antigen-expressing IL-2Rβ+ cells, such as CD8+ T cells, over antigen-non expressing IL-2Rβ+ cells, such as NK cells, by at least 10 fold, 50 fold, 100 fold, or at least 200 fold. In some embodiments, disclosed fusion proteins activate antigen-expressing IL-2Rβ+ cells more than 50 fold, 100 fold, or at least 200 fold, e.g., compared to a fusion protein comprising the said IL-2 mutant polypeptide and a control antibody not binding to any antigens expressed on said cells. Said cell activation by the IL-2 fusion protein is determined in an in vitro assay by measuring the expression of pSTAT5 or the cell proliferation marker Ki67 in said cells following treatment with said IL-2 fusion protein.

In summary, the present disclosure accomplishes to reduce the pleiotropic effects of IL-2 on every immune cell expressing the IL-2R complex down to a subset of effects by reducing the effects of IL-2 to certain immune cell subsets of interest, such as CD8+ T cells. Such reduction aims to reduce the toxicity of IL-2 polypeptides when administered as therapeutics by directing their action on subsets of T cells that contain tumor antigen-specific CD8+ T cells or viral antigen-specific CD8+ T cells thus sparring: 1) T cells that may not contribute to efficacy; or 2) innate lymphocytes that express receptors for IL-2 and are systemically distributed and may contribute to toxicity; 3) other immune cells that can act as a sink for IL-2 or negatively contribute to efficacy.

### BRIEF DESCRIPTION OF THE DRAWINGS

FIGS. 1A-1D show the amino acid sequences of mature IL-2 (FIG. 1A; SEQ ID NO:1), IL-2Rα(FIG. 1B; SEQ ID NO:2), IL-2Rβ (FIG. 1C; SEQ ID NO:3) and IL-2Rγ (FIG. 1D; SEQ ID NO:4) polypeptides.

FIG. 2 shows the amino acid sequence of wild-type mature IL-2 polypeptide (SEQ ID NO:1). “X” denotes the amino acid substituted in the sequence of wild-type IL-2 polypeptide for another amino acid to generate mutant IL-2 polypeptides of the present disclosure.

FIG. 3 shows the general mechanism for how targeted fusions of mutant IL-2 polypeptides with CD8 or PD1 antigen binding molecules, and untargeted fusions with mutant IL-2 polypeptides work to stimulate cells expressing or not expressing CD8 or PD1 antigens.

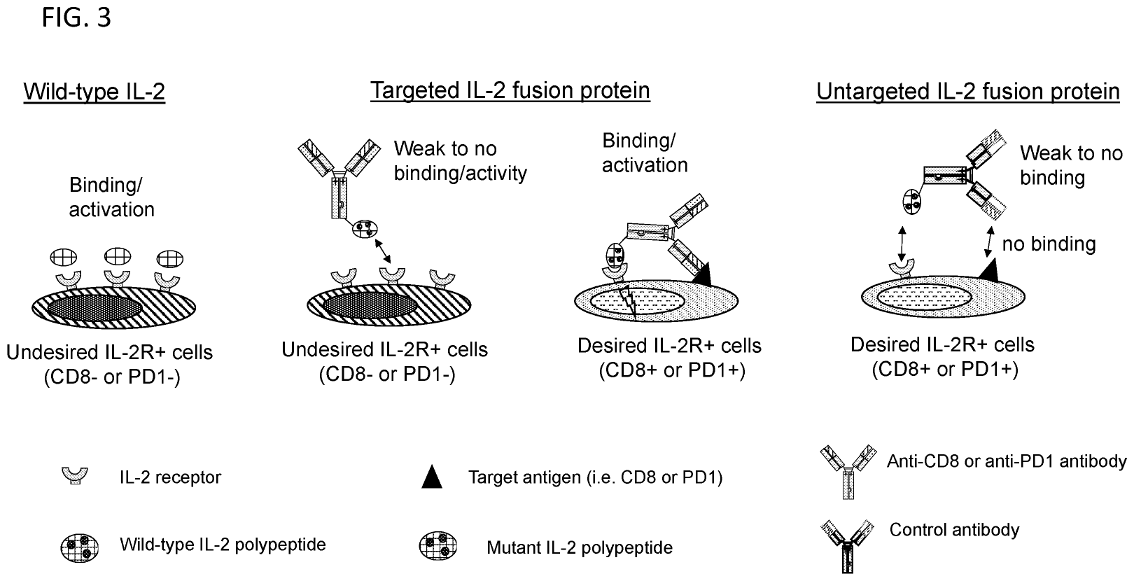

FIG. 4 depicts three different fusion protein formats (formats A, B, and C), in accordance with some embodiments.

FIG. 5 shows the activation of STAT5 in different mouse splenic cell subsets stimulated with therapeutic human IL-2 (left) and an IL-2 variant with no binding to IL-2Rα and wild type binding, to IL-2Rβ and IL-2Rγ (right). STAT5 activation in splenic subsets was measured by flow cytometry. Activation of STAT5 in splenic cell subsets stimulated with IL-2 is shown at left. Activation of STAT5 in splenic cell subsets stimulated with a fusion of a previously published IL-2 variant (IL-2v; see Klein et al, Oncoimmunol. 2017; 6 (3); e1277306) that does not bind to IL-2Ra and control antibody (xHA), xHA-IL-2v, is shown at right. NK cells were found to be more sensitive to IL-2 and IL-2 variant with reduced binding to CD25/IL2Rα than CD8 T cells.

FIGS. 6A & 6B show the NK-cell induced toxicity of IL-2 variant with reduced binding to CD25 in mice. B6 mice of 8-10 weeks of age were injected subcutaneously with a single dose of indicated compounds and their body weights recorded daily. FIG. 6A shows the weight recordings for mice treated with xHA-IL-2v co-dosed with anti-PD1 (xPD1) at 2.5 mg/kg. FIG. 6B shows the weight recordings for mice treated with the same IL-2v fused to an antibody targeting an antigen expressed in the tumor (tumor antigen/TAg) TAg-IL-2v. TAg-IL-2v was dosed alone at 5 mg/kg. NK cells were depleted with anti-NK1.1 antibody (PK136 clone) at 200 mg/mouse i.p. Depleting antibody was injected two days prior to TAg-IL-2v dosing and one day after dosing to maintain depletion. NK cells induced toxicity, which manifested as body weight loss in mice treated with IL-2 variant with reduced binding CD25/IL2Ra.

FIG. 7 shows the determination of binding of anti-mouse CD8 antibodies to CD8+ T cells. Fresh splenocytes were incubated with the indicated antibodies for 2 hours at 4° C. The cells were then stained with antibodies against CD3, CD4, CD8 and anti-hFc. anti-hFc was used to measure the binding of CD8-IL2 fusion containing hFc. Cells were washed and analyzed by flow cytometry. Mean fluorescence intensity (MFI) of staining with anti-hFc is used to denote binding. xmCD8ab2 (published clone YTS156.7.7) had higher affinity than xmCD8ab1 (published clone 2.43). xCD8ab2.1 is a lower affinity variant of xCD8ab2 that was generated by introducing two mutations in xCD8ab2.

FIG. 8 shows the MHC blocking status for anti-mouse CD8 antibodies. CD8+ T cells were purified from splenocytes from OT-I mice and co-cultured with EL-4-OVA expressing line (E.G7-OVA, CRL-2113; ATCC), at 100,000 cells each for 24 hr. Cells were analyzed for upregulation of activation markers such as CD25 and CD69 by cell surface staining and flow cytometry. Both xCD8ab1 and xCD8ab2 blocked T cell activation, as measured by % of cells expressing CD25. xCD8ab2 blocked T cell activation more potently, correlating with its higher binding affinity to CD8.

FIG. 9 shows the selective targeting of CD8 T cells over other immune cells expressing IL-2R by human IL-2 muteins fused to CD8 antibodies. IL-2 muteins were fused to previously published anti-mouse CD8 antibody, xmCD8ab1 (2.43 clone), in format B (depicted in the diagram at left). STAT5 activation in mouse splenocytes was measured by flow cytometry. IL-2 mutein variants fused to CD8 antibodies selectively targeted CD8+ T cells (top left graph) over other immune cells expressing IL-2R, such as NK cells identified as CD3-CD49b+ (top right), CD4+CD25−Tconv cells (bottom left), and CD4+CD25+ Treg cells (bottom right).

FIG. 10 shows the efficacy of a single dose of CD8-IL-2 over a single dose of TAg-IL-2v in combination with anti-PD1 in a B16 cold tumor model. C57BL6 mice were implanted with 5×10\hat{ }5 cells (100 μL) of cultured B16.F10 cells (ATCC, CRL-6475) subcutaneously above the rear hind leg. Tumor volume was measured until it reached 60-120 mm³ at approximately 8 days post implantation (TV=width*width*length*0.5). Each mouse was weighed and dosed subcutaneously under the scruff of the neck with the indicated compounds (10 mice/group): PBS (top left), xmCD8ab1-IL2m10 at 1 mg/kg (top middle), xmCD8ab2-IL2m10 at 1 mg/kg (top right), xPD1 at 5 mg/kg (bottom left), TAg-IL-2v at 1 mg/kg (bottom middle), or TAg-IL-2v at 3 mg/kg (bottom right). Tumor volume and body weight were measured every 3-4 days until either the end of study (30-40 days post initial dose) or until a max tumor volume (2000 mm³) was reached. Complete regression of tumor (CR) and partial regression of tumor or slower growth defined by <100 mm³ by day 14 post dose (PR) are denoted when applicable. All mice except those in the PBS and xPD1 groups were co-administered with anti-PD1. xmCD8-IL2m10 performed better than TAg-IL-2v in the B16 cold tumor model in combination with anti-PD1.

FIGS. 11A & 11B show the induction of CD8 T cell accumulation in the blood and tumor of mice with B16 tumors treated with a single dose CD8-IL-2. B6 mice were injected with B16 tumor cells and tumors allowed to grow to 200-250 mm³ before they were dosed with indicated IL-2 fusions at 1 mg/kg together with 5 mg/kg of xPD1. Cells from tumors and blood were collected and profiled by flow cytometry to detect CD8+ T cells and NK cells (NK1.1+CD3-), as indicated. FIG. 11A shows the immune cell counts in the blood, while FIG. 11B shows the immune cells count in the tumor. More CD8+ T cells were observed in the tumor with xmCD8-IL2m10 than xHA-IL-2v.

FIGS. 12A-12C show the performance of a single dose of CD8-IL-2 and single dose of TAg-IL-2v in a CT26 tumor model. BALB/c female mice were implanted with 2×105 cells (100 μL) of cultured CT26.wt cells (ATCC, CRL-2638) subcutaneously above the rear hind leg. Tumor volume was measured until it reached 60-120 mm³ was reached at approximately 8 days post implantation (TV=width*width*length*0.5). Each mouse was then weighed and dosed subcutaneously with the indicated compound (9 mice/group): PBS (FIG. 12A), TAg-IL-2v at 2 mg/kg (FIG. 12B), or xmCD8ab2-IL2m4 at 0.3 mg/kg (FIG. 12C). Tumor volumes and body weights were measured every 3-4 days until either the end of the study (30 days post initial dose) or until a max tumor volume (2000 mm³) was reached. Complete regression of tumor (CR) is denoted when applicable. xmCD8-IL2m4 performed better than TAg-IL-2v in CT26 tumor model.

FIG. 13 shows the impact of CD8 antibody affinity on fusion potency in vitro. Cells were treated with IL-2 mutein IL-2m4 fused to xmCD8ab2 or its lower affinity variant xmCD8ab2.1 in format C. STAT5 activation in CD8+ T cells (left) and NK cell (right) was measured by flow cytometry. xmCD8ab2.1-IL2m4 had lower potency and lower selectivity for CD8 T cells over NK cells compared to xmCD8ab2-IL-2m4.

FIG. 14 shows the in vivo expansion of CD8+ T cells treated with xmCD8ab2-IL2m4 and xmCD8ab2.1-IL2m4. Naïve B6 mice were treated with the indicated compounds at 1 mg/kg and blood collected on day 5 post dose. Cells were stained with lineage markers to identify CD8+ T cells and NK cells and profiled by flow cytometry. Both xmCD8ab2-IL2m4 and xmCD8ab2.1-IL2m4 expanded CD8 T cells in vivo. Both fusions induced higher in vivo expansion of CD8 T cells than NK cells.

FIGS. 15A & 15B show the characterization of xmCD8-IL-2 muteins with no binding to IL-2Rα and one additional mutation fused to a high affinity CD8 antibody in a STAT5 assay. Splenocytes from B6 mice bearing B16 tumors incubated with indicated protein then stained for cell surface markers (CD3, CD4, CD8, CD25, CD49b) and for intracellular phospho-STAT5 (pSTAT5). Cells were analyzed by flow cytometry. Data show mean fluorescence intensity (MFI) for STAT5 in the indicated cell subsets. FIG. 15A shows the activation of STAT5 in CD8+ T cells, while FIG. 15B shows the activation of STAT5 in NK cells (defined as CD3-CD49b+). Certain IL-2 mutations lowered binding of IL-2 muteins fused to anti-CD8 antibodies to IL2Rβ/γ-expressing cells while maintaining their higher potency on CD8+ T cells over other IL2Rβ/γ-expressing cells not expressing CD8.

FIGS. 16A & 16B shows the characterization of xmCD8-IL-2 muteins with no binding to IL-2Rα and one additional mutation fused to a low affinity CD8 antibody variant in a STAT5 assay. Splenocytes from B6 mice bearing B16 tumors incubated with indicated protein then stained for cell surface markers (CD3, CD4, CD8, CD25, CD49b) and for intracellular STAT5. Cells were analyzed by flow cytometry. Data show mean fluorescence intensity (MFI) for STAT5 in the indicated cell subsets. FIG. 16A shows the activation of STAT5 in CD8+ T cells, while FIG. 16B shows the activation of STAT5 in NK cells. Certain IL-2 mutations lowered binding of IL-2 muteins fused to low affinity anti-CD8 antibodies to IL2Rβ/γ-expressing cells while maintaining their higher potency on CD8+ T cells over other IL2Rβ/γ-expressing cells not expressing CD8.

FIGS. 17A & 17B show the characterization of xmCD8-IL-2 muteins with no binding to IL-2Rα and one additional mutation fused to a high affinity CD8 antibody in an assay detecting expression of a cell proliferation marker (Ki67). Splenocytes from B6 mice bearing B16 tumors incubated with indicated protein then stained for cell surface markers (CD3, CD4, CD8, CD25, CD49b) and for the intracellular marker of proliferation, Ki67, a downstream signaling event from IL2Rβ/γ and STAT5. Data show % of cells in the indicated cell subset positive for proliferation marker Ki67. FIG. 17A shows the percent of Ki67-positive CD8+ T cells, while FIG. 17B shows the percent of Ki67-positive NK cells.

FIGS. 18A & 18B shows the characterization of xmCD8-IL-2 muteins with no binding to IL-2Rα and one additional mutation fused to a low affinity CD8 antibody in the Ki67 assay. Splenocytes from B6 mice bearing B16 tumors incubated with indicated protein then stained for cell surface markers (CD3, CD4, CD8, CD25, CD49b) and for Ki67. Data show % of cells in the indicated cell subset positive for proliferation marker Ki67. FIG. 18A shows the percent of Ki67-positive CD8+ T cells, while FIG. 18B shows the percent of Ki67-positive NK cells.

FIG. 19 depicts the summary of potencies on CD8 T cells and NK cells for representative molecules and their selectivity for CD8 T cells over NK cells. CD8 and NK cell activation for each molecule are indicated on the same graph. Difference in potency on CD8 vs NK cells is indicated with a double arrow. The graph summarizes characterization data for generated CD8-IL2 fusions that have a range of selectivity for CD8+ T cells over NK cells. xmCD8ab2-IL2m4 (top left) and xmCD8ab2-IL2m4.2 (top right) had the highest selectivity (>1000×), followed by xmCD8ab2.1-IL2m4 (˜50-100×; bottom left) and xmCD8ab2.1-IL2m4.1 with the lowest (˜10×; bottom right).

FIG. 20 depicts the CD8-IL-2 fusion properties with best efficacy. Four representative CD8-IL2 fusions (as indicated) with varying degrees of selectivity for CD8 T cells over NK cells were tested in a B16 tumor model. Number of mice with complete regression out of total mice (CR) are indicated for each panel. All mice were dosed with 1 mg/kg of the indicated fusions together with 5 mg/kg of anti-PD1. Dosing above 1 mg/kg can induce NK cell activation due to the binding of IL-2 mutein to IL-2Rβγ on NK cells, thereby inducing body weight loss and toxicity. CD8-IL-2 performed better than TAg-IL-2v at a lower dose. CD8-IL2 fusion with the lowest selectivity for CD8 T cells had the least efficacy in the B16 model, approaching that observed for TAg-IL-2v in FIG. 10 with only 1 mouse out of 10 showing complete tumor regression. Selectivity of >10× was required for best efficacy, therapeutic index, and >40% tumor free mice.

FIG. 21 shows the expansion of tumor antigen-specific CD8+ T cells and total CD8+ T cells and NK cells by treatment with CD8-IL-2. B6 mice were injected with B16 tumor cells and tumors allowed to grow to 200-250 mm3 before they were dosed with indicated IL-2 fusions at 1 mg/kg together with 5 mg/kg of xPD1. Tumors were removed day 5 post dose, digested to single cells and profiled by flow cytometry to detect CD8+ T cells and NK cells (NK1.1+CD3-). Cells were also stained with p15E tetramer (TB-M507-2, MBL) according to manufacturer's protocol to detect T cells that recognize p15E tumor antigen. Data are expressed as cell counts per 10⁶ cells isolated from each tumor. Both xmCD8ab2-IL2m4.2 and xmCD8ab2.1-IL2m4 induced ˜15× expansion of total intratumoral CD8+ T cells and 5-17× expansion in p15E tumor antigen-specific T cells.

FIG. 22 shows the potency of a bivalent low affinity CD8 antibody IL-2 fusion and a monovalent high affinity CD8 antibody IL-2 fusion. Splenocytes from B6 mice bearing B16 tumors were incubated with indicated protein for 30 min in RPMI media after which cells were stained for cell surface markers (CD3, CD4, CD8, CD25, CD49b) and for intracellular phospho-STAT5. The bivalent low affinity fusion had a similar potency to that of the high affinity monovalent fusion as measured by percentage of cells positive for pSTAT5. IL-2m4.2 fusion fused to high affinity xmCD8ab2 antibody (in format C) or to bivalent xmCD8ab2.1 antibody (in format A) had similar potencies on CD8+ T cells and much greater potency than monovalent xmCD8ab2.1-IL-2m4 (format C) fusion.

FIG. 23 show the efficacy of a bivalent C-terminal format (format A) fusion in the B16 tumor model. Mice were dosed PBS as control or with 1 mg/kg of the indicated fusions together with 5 mg/kg of anti-PD1 (9 per group). Bivalent C-terminal format (format A) was also very efficacious. IL-2m4.2 fusion fused to high affinity xmCD8ab2 antibody in format C (FIG. 20) or to bivalent xmCD8ab2.1 antibody in format A (FIG. 23) had similar in vivo efficacy.

FIG. 24 shows blocking of CD8 T cell activation by CD8 antibodies. CD8+ T cells were purified from splenocytes from OT-I mice and co-cultured with EL-4-OVA line (ATCC), at 100,000 cells each for 24 hr. Cells were analyzed for upregulation of activation markers such as CD25 and CD69 by cell surface staining and flow cytometry. Certain CD8 antibodies did not block CD8 T cell activation. xmCD8ab3 antibody (comprising a VH domain comprising the sequence of SEQ ID NO:16 and a VL domain comprising the sequence of SEQ ID NO:17) did not block CD8 T cell activation even at 200 nM concentration. The xmCD8ab3 antibody was of the bivalent format.

FIG. 25 shows the comparison of in vitro potency of xmCD8ab2 and xmCD8ab3 fusions, as indicated. Splenocytes from B6 mice bearing B16 tumors were incubated with indicated protein for 30 min in RPMI media after which cells were stained for cell surface markers (CD3, CD4, CD8, CD25, CD49b) and for intracellular pSTAT5. Both xmCD8ab2-IL2m4.2 and xmCD8ab3-IL2m4.2 exhibited similar activity on CD8+ T cells in vitro with greater potency than TAg-IL-2v.

FIGS. 26A & 26B show the in vivo effect of an MHC non-blocking anti-CD8 antibody fused to IL-2 mutein in the B16 tumor model. Mice were dosed with PBS or 0.3 mg/kg (FIG. 26A) or 1 mg/kg (FIG. 26B) of the indicated fusion together with 5 mg/kg of anti-PD1. IL-2m4.2 fusion fused to MHC non-blocking xmCD8ab3 antibody in format C (FIG. 26B) was much more efficacious than IL-2m4.2 fusion fused to MHC blocking xmCD8ab2 antibody in format C (FIG. 26A).

FIG. 27 shows the fusion of IL-2 muteins which preferentially target PD1+ T cells over PD1− T cells. B16 tumors 300-600 mm³ in size were removed from mice and digested to single cells. CD45+ cells were purified (Miltenyi's LS columns according to manufacturer's protocol) and stimulated with indicated fusion proteins for 30 min. Cells were stained for cell surface markers (CD3, CD4, CD8, CD25, CD49b, and PD1) and for intracellular phospho-STAT5. Fusion of IL-2 mutein IL2m10 with anti-PD1 antibody preferentially targeted PD1+ T cells over PD1− T cells; however both CD8+PD1+ T cells and CD4+CD25+PD1+ Treg cells were targeted.

### DETAILED DESCRIPTION

### Definitions

“Immune cells” as used here are cells of the immune system that react to organisms or other entities that are deemed foreign to the immune system of the host. They protect the host against foreign pathogens, organisms and diseases. Immune cells, also called leukocytes, are involved in both innate and adaptive and immune responses to fight pathogens. Innate immune responses occur immediately upon exposure to pathogens without additional priming or learning processes. Adaptive immune processes require initial priming, and subsequently create memory, which in turn leads to enhanced responsiveness during subsequent encounters with the same pathogen Innate immune cells include, but are not limited to monocytes, macrophages, dendritic cells, innate lymphoid cells (ILCs) including natural killer (NK) cells, neutrophils, megakaryocytes, eosinophils and basophils. Adaptive immune cells include B and T lymphocytes/cells. T cells subsets include, but are not limited to, alpha beta CD4+ T (naïve CD4+, memory CD4+, effector memory CD4+, effector CD4+, regulatory CD4+), and alpha beta CD8+ T (naïve CD8+, memory CD8+, effector memory CD8+, effector CD8+). B cell subsets include, but is not limited to, naïve B, memory B, and plasma cells. NK T cells and T gamma delta (Tγδ) cells exhibit properties of both innate and adaptive lymphocytes.

“T cells” or “T lymphocytes” are immune cells that play a key role in the orchestration of immune responses in health and disease. Two major T cell subsets exist that have unique functions and properties: T cells that express the CD8 antigen (CD8⁺ T cells) are cytotoxic or killer T cells that can lyse target cells using the cytotoxic proteins such as granzymes and perforin; and T cells that express the CD4 antigen (CD4⁺ T cells) are helper T cells that are capable of regulating the function of many other immune cell types including that of CD8⁺ T cells, B cells, macrophages etc. Furthermore, CD4⁺ T cells are further subdivided into several subsets such as: T regulatory (Treg) cells that are capable of suppressing the immune response, and T helper 1 (Th1), T helper 2 (Th2), and T helper 17 (Th17) cells that regulate different types of immune responses by secreting immunomodulatory proteins such as cytokines. T cells recognize their targets via alpha beta T cell receptors that bind to unique antigen-specific motifs and this recognition mechanism is generally required in order to trigger their cytotoxic and cytokine-secreting functions. “Innate lymphocytes” can also exhibit properties of CD8⁺ and CD4⁺ T cells, such as the cytotoxic activity or the secretion of Th1, Th2, and Th17 cytokines. Some of these innate lymphocyte subsets include NK cells and ILC1, ILC2, and ILC3 cells; and innate-like T cells such as Tγδ cells; and NK T cells. Typically, these cells can rapidly respond to inflammatory stimuli from infected or injured tissues, such as immunomodulatory cytokines, but unlike alpha beta T cells, they can respond without the need to recognize antigen-specific patterns.

“Cytokine” is a form of immunomodulatory polypeptide that mediates cross-talk between initiating/primary cells and target/effector cells. It can function as a soluble form or cell-surface associated to bind the “cytokine receptor” on target immune cells to activate signaling. “Cytokine receptor” as used here is the polypeptide on the cell surface that activates intracellular signaling upon binding the cytokine on the extracellular cell surface. Cytokines includes, but are not limited to, chemokines, interferons, interleukins, lymphokines, and tumor necrosis factors. Cytokines are produced by a wide range of cells, including immune cells, endothelial cells, fibroblasts, and stromal cells. A given cytokine may be produced by more than one cell type. Cytokine are pleiotropic; since the receptors are expressed on multiple immune cell subsets, one cytokine can activate the signaling pathway in multiple cells. However, depending on the cell type, the signaling events for a cytokine can result in different downstream cellular events such as activation, proliferation, survival, apoptosis, effector function and secretion of other immunomodulatory proteins.

“Amino acid” as used here refers to naturally occurring carboxy α-amino acids comprising alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

“Polypeptide” or “protein” as used here refers to a molecule where monomers (amino acids) are linearly linked to one another by peptide bonds (also known as amide bonds). The term “polypeptide” refers to any chain of two or more amino acids and does not refer to a specific length of the product. Thus, peptides, dipeptides, tripeptides, oligopeptides, “protein”, “amino acid chain”, or any other term used to refer to a chain of two or more amino acids, are included within the definition of “polypeptide”, and the term “polypeptide” may be used instead of, or interchangeably with any of these terms. The term “polypeptide” is also intended to refer to the products of A polypeptide may be derived from a natural biological source or produced by recombinant technology but is not necessarily translated from a designated nucleic acid sequence. It may be generated in any manner, including by chemical synthesis. Polypeptides normally have a defined three-dimensional structure, but they do not necessarily have such structure. A polypeptide of the present disclosure may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides with a defined three-dimensional structure are referred to as folded, and polypeptides which do not possess a defined three-dimensional structure, but rather can adopt many different conformations and are referred to as unfolded. Polypeptides may further form multimers such as dimers, trimers and higher oligomers, i.e. consisting of more than one polypeptide molecule. Polypeptide molecules forming such dimers, trimers etc. may be identical or non-identical. The corresponding higher order structures of such multimers are, consequently, termed homo- or heterodimers, homo- or heterotrimers etc. The terms “polypeptide” and “protein” also refer to modified polypeptides/proteins wherein the post-expression modification is affected including without limitation glycosylation, acetylation, phosphorylation, amidation, derivatization by known protecting/blocking groups, proteolytic cleavage, or modification by non-naturally occurring amino acids.

“Residue” as used herein is meant a position in a protein and its associated amino acid identity. For example, Leu 234 (also referred to as Leu234 or L234) is a residue at position 234 in the human antibody IgG1.

“wild-type” herein means an amino acid sequence or a nucleotide sequence that is found in nature, including allelic variations. A wild-type protein has an amino acid sequence or a nucleotide sequence that has not been intentionally modified.

“Substitution” or “mutation” refers to a change to the polypeptide backbone wherein an amino acid occurring naturally in the wild-type sequence of a polypeptide is substituted to another amino acid not naturally occurring at the same position in the said polypeptide. Preferably, a mutation or mutations are introduced to modify polypeptide's affinity to its receptor thereby altering its activity such that it becomes different from the affinity and activity of the wild-type cognate polypeptide. Mutations can also improve polypeptide's biophysical properties. Amino acid mutations can be generated using genetic or chemical methods well known in the art. Genetic methods may include site-directed mutagenesis, PCR, gene synthesis and the like. It is contemplated that methods of altering the side chain group of an amino acid by methods other than genetic engineering, such as chemical modification, may also be useful.

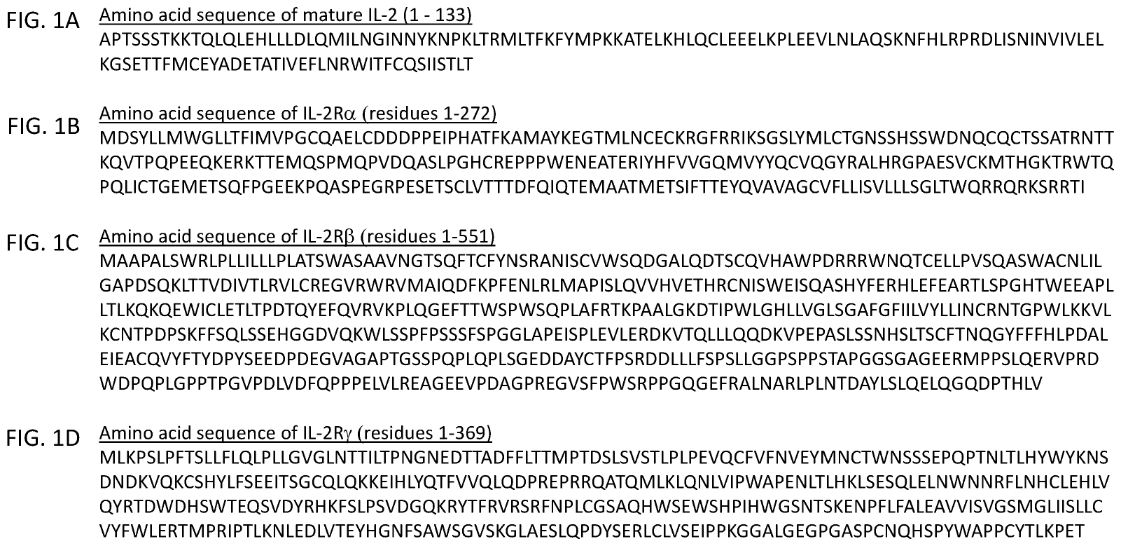

“Interleukin-2” or “IL-2” as used here refers to any native human IL-2, unless otherwise indicated. “IL-2” encompasses unprocessed IL-2 as well as “mature IL-2” which is a form of IL-2 that results from processing in the cell. The sequence of “mature IL-2” is depicted in FIG. 1A. One exemplary form of unprocessed human IL-2 comprises of an additional N-terminal amino acid signal peptide attached to mature IL-2. “IL-2” also includes but is not limited to naturally occurring variants of IL-2, e.g. allelic or splice variants or variants. The amino acid sequence of an exemplary human IL-2 is described under UniProt P60568 (IL2 HUMAN).

“Affinity” or “binding affinity” refers to the strength of the sum total of non-covalent interactions between a single binding site of a molecule (e.g. an antibody) and its binding partner (e.g. an antigen). Unless indicated otherwise, as used herein, “binding affinity” refers to intrinsic binding affinity which reflects a 1:1 interaction between members of a binding pair (e.g. antibody and antigen). The affinity can generally be represented by the dissociation constant (K_{D}), which is the ratio of dissociation and association rate constants (koff and kon, respectively). Thus, equivalent affinities may comprise different rate constants, as long as the ratio of the rate constants remains the same. Affinity can be measured by common methods known in the art, such as enzyme-linked immunosorbent assay (ELISA), surface plasmon resonance (SPR) technologies (e.g. BIAcore), BioLayer Interferometry (BLI) technologies (e.g. Octet) and other traditional binding assays (Heeley, Endocr Res 28, 217-229 (2002).

“Binding” or “Specific binding” as used here, refers the ability of a polypeptide or an antigen binding molecule to selectively interact with the receptor for the polypeptide or target antigen, respectively, and this specific interaction can be distinguished from non-targeted or undesired or non-specific interactions. Examples of specific binding include but are not limited to IL-2 cytokine binding to its specific receptors (e.g. IL-2Rα, IL-2Rβ and IL-2Rγ) and an antigen binding molecule binding to a specific antigen (e.g. CD8 or PD-1).

“Mutant IL-2 polypeptide” refers to IL-2 polypeptide that has reduced affinity to its receptor wherein such decreased affinity will result in reduced biological activity of the mutant. Reduction in affinity and thereby activity can be obtained by introducing a small number of amino acid mutations or substitutions. The mutant IL-2 polypeptides can also have other modifications to the peptide backbone, including but not limited to amino acid deletion, permutation, cyclization, disulfide bonds, or the post-translational modifications (e.g. glycosylation or altered carbohydrate) of a polypeptide, chemical or enzymatic modifications to the polypeptide (e.g. attaching PEG to the polypeptide backbone), addition of peptide tags or labels, or fusion to proteins or protein domains to generate a final construct with desired characteristics, such as reduced affinity to IL-2Rγγ. Desired activity may also include improved biophysical properties compared to the wild-type IL-2 polypeptide. Multiple modifications may be combined to achieve desired activity modification, such as reduction in affinity or improved biophysical properties. As a non-limiting example, amino acid sequences for consensus N-link glycosylation may be incorporated into the polypeptide to allow for glycosylation. Another non-limiting example is that a lysine may be incorporated onto the polypeptide to enable pegylation. Preferably, a mutation or mutations are introduced to the polypeptide to modify its activity.

“Targeting moiety” and “antigen binding molecule” as used here refers in its broadest sense to a molecule that specifically binds an antigenic determinant. A targeting moiety or antigen binding molecule may be a protein, carbohydrate, lipid, or other chemical compound. It includes, but is not limited to, antibody, antibody fragments (Chames et al, 2009; Chan & Carter, 2010; Leavy, 2010; Holliger & Hudson, 2005), scaffold antigen binding proteins (Gebauer and Skerra, 2009; Stumpp et al, 2008), single domain antibodies (sdAb), minibodies (Tramontano et al, 1994), the variable domain of heavy chain antibodies (nanobody, VHH), the variable domain of the new antigen receptors (VNAR), carbohydrate binding domains (CBD) (Blake et al, 2006), collagen binding domain (Knight et al, 2000), lectin binding proteins (Tetranectin), collagen binding proteins, adnectin/fibronectin (Lipo\check{v}sek, 2011), a serum transferrin (trans-body), Evibody, Protein A-derived molecule, such as Z-domain of Protein A (Affibody) (Nygren et al, 2008), an A-domain (Avimer/Maxibody), alphabodies (WO2010066740), Avimer/Maxibody, designed ankyrin-repeat domains (DARPins) (Stumpp et al, 2008), anticalins (Skerra et al, 2008), a human gamma-crystallin or ubiquitin (Affilin molecules), a kunitz type domain of human protease inhibitors, knottins (Kolmar et al, 2008), linear or constrained peptide with or without fusion to extend half-life e.g. (Fc fusion—Peptibody) (Rentero Rebollo & Heinis, 2013; EP 1144454 B2; Shimamoto et al, 2012; U.S. Pat. No. 7,205,275 B2), constrained bicyclic peptides (US 2018/0200378 A1), aptamer, engineered CH2 domains (nanoantibodies; Dimitrov, 2009)) and engineered CH3 domain “Fcab” domains (Wozniak-Knopp et al, 2010).

The term “antibody” and “immunoglobulin” are used interchangeably and herein are used in the broadest sense and encompasses various antibody structures, including but not limited to monoclonal antibodies (e.g., full length or intact monoclonal antibodies), polyclonal antibodies, multispecific antibodies (e.g. bispecific antibodies), antibody fragments and single domain antibody (as described in greater detail herein), so long as they exhibit the desired antigen binding activity.

Antibodies (immunoglobulins) refers to a protein having a structure substantially similar to a native antibody structure. “Native antibodies” refer to naturally occurring immunoglobulin molecules with varying structures. For example, native immunoglobulins of the IgG class are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a constant light (CL) domain, also called a light chain constant region. The subunit structures and three-dimensional configurations of the different classes of immunoglobulins are well known and described generally, for example, in Abbas et al., 2000, Cellular and Mol, and Kindt et al., Kuby Immunology, 6th ed., W.H. Freeman and Co., page 91 (2007). Antibodies (immunoglobulins) are assigned to different classes, depending on the amino acid sequences of the heavy chain constant domains. There are five major classes of antibodies: α (IgA), δ (IgD), ϵ (IgE), γ (IgG), or μ (IgM), some of which may be further divided into subtypes, e.g. γ1 (IgG1), γ2 (IgG2), γ3 (IgG3), γ4 (IgG4), α1 (IgA1) and α2 (IgA2). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

“Fc” or “Fc region” or “Fc domain” as used herein refers to the C-terminal region of an antibody heavy chain that contains at least a portion of the constant region. The term includes native sequence Fc regions and variant Fc regions. An Fc can refer to the last two constant region immunoglobulin domains (e.g., CH2 and CH3) of IgA, IgD, and IgG, the last three constant region immunoglobulin domains of IgE and IgM, and optionally, all or a portion of the flexible hinge N-terminal to these domains. For IgA and IgM, Fc may include the J chain. An IgG Fc region comprises an IgG CH2 and an IgG CH3 domain and in some cases, inclusive of the hinge. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991. The “hinge” region usually extends from amino acid residue at about position 216 to amino acid residue at about position 230. The hinge region herein may be a native hinge domain or variant hinge domain. The “CH2 domain” of a human IgG Fc region usually extends from an amino acid residue at about position 231 to an amino acid residue at about position 340. The CH2 domain herein may be a native sequence CH2 domain or variant CH2 domain. The “CH3 domain” comprises the stretch of residues C-terminal to a CH2 domain in an Fc region, from an amino acid residue at about position 341 to an amino acid residue at about position 447 of an IgG. The CH3 region herein may be a native sequence CH3 domain or a variant CH3 domain (e.g. a CH3 domain with an introduced “protuberance” (“knob”) in one chain thereof and a corresponding introduced “cavity” (“hole”) in the other chain thereof; see U.S. Pat. No. 5,821,333, expressly incorporated herein by reference). Thus, the definition of “Fc domain” includes both amino acids 231-447 (CH2-CH3) or 216-447 (hinge-CH2-CH3), or fragments thereof. An “Fc fragment” in this context may contain fewer amino acids from either or both of the N- and C-termini but still retains the ability to form a dimer with another Fc domain or Fc fragment as can be detected using standard methods, generally based on size (e.g. non-denaturing chromatography, size exclusion chromatography, etc.). Human IgG Fc domains are of particular use in the present disclosure, and can be the Fc domain from human IgG1, IgG2 or IgG4.

A “variant Fc domain” or “Fc variant” or “variant Fc” contains amino acid modifications (e.g. substitution, addition, and deletion) as compared to a parental Fc domain. The term also includes naturally occurring allelic variants of the Fc region of an immunoglobulin. In general, variant Fc domains have at least about 80, 85, 90, 95, 97, 98 or 99 percent identity to the corresponding parental human IgG Fc domain (using the identity algorithms discussed below, with one embodiment utilizing the BLAST algorithm as is known in the art, using default parameters). Alternatively, the variant Fc domains can have from 1, 2, 3, 4, 5, 6, 7, 8, 9, 10, 11, 12, 13, 14, 15, 16, 11, 12, 13, 14, 15, 16, 17, 18, 19 or 20 amino acid modifications as compared to the parental Fc domain. For example, one or more amino acids can be deleted from the N-terminus or C-terminus of the Fc region of an immunoglobulin without substantial loss of biological function. Additionally, as discussed herein, the variant Fc domains herein still retain the ability to form a dimer with another Fc domain as measured using known techniques as described herein, such as non-denaturing gel electrophoresis.

“Fc gamma receptor”, “FcγR” or “Fc gamma R” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIb-NA1 and FcγRIIb-NA2) (Jefferis et al., 2002, Immunol Lett 82:57-65, entirely incorporated by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes.

By “effector function” as used herein is meant a biochemical event that results from the interaction of an antibody Fc region with an Fc receptor or ligand, which vary with the antibody isotype. Effector functions include but are not limited to antibody-dependent cell-mediated cytotoxicity (ADCC), antibody-dependent cell-mediated phagocytosis (ADCP), complement-dependent cytotoxicity (CDC), cytokine secretion, immune complex-mediated antigen uptake by antigen presenting cells, down regulation of cell surface receptors (e.g. B cell receptor), and B cell activation. “Antibody-dependent cell-mediated cytotoxicity” or “ADCC” refer to a cell-mediated reaction in which nonspecific cytotoxic cells that express FcRs (such as Natural Killer (NK) cells, neutrophils, and macrophages) recognize bound antibody on a target cell and subsequently cause lysis of the target cell. ADCC is correlated with binding to FcγRIIIa; increased binding to FcγRIIIa leads to an increase in ADCC activity. To assess ADCC activity of a molecule of interest, an in vitro ADCC assay such as that described in U.S. Pat. No. 5,500,362 or 5,821,337 may be performed. “ADCP” or antibody dependent cell-mediated phagocytosis as used herein is meant the cell-mediated reaction wherein nonspecific cytotoxic cells that express FcγRs recognize bound antibody on a target cell and subsequently cause phagocytosis of the target cell.

“Fc null” and “Fc null variant” are used interchangeably and used herein to describe a modified Fc which have reduced or abolished effector functions. Such Fc null or Fc null variant have reduced or abolished to FcγRs and/or complement receptors. Preferably, such Fc null or Fc null variant has abolished effector functions. Exemplary methods for the modification include but not limited to chemical alteration, amino acid residue substitution, insertion and deletions. Exemplary amino acid positions on Fc molecules where one or more modifications were introduced to decrease effector function of the resulting variant (numbering based on the EU numbering scheme) at position i) IgG1: C220, C226, C229, E233, L234, L235, G237, P238, S239 D265, 5267, N297, L328, P331, K322, A327 and P329, ii) IgG2: V234, G237, D265, H268, N297, V309, A330, A331, K322 and iii) IgG4: L235, G237, D265 and E318. Exemplary Fc molecules having decreased effector function include those having one or more of the following substitutions: i) IgG1: N297A, N297Q, D265A/N297A, D265A/N297Q, C220S/C226S/C229S/P238S, S267E/L328F, C226S/C229S/E233P/L234V/L235A, L234F/L235E/P331S, L234A/L235A, L234A/L235A/G237A, L234A/L235A/G237A/K322A, L234A/L235A/G237A/A330S/A331S, L234A/L235A/P329G,E233P/L234V/L235A/G236del/S239K, E233P/L234V/L235A/G236del/S267K, E233P/L234V/L235A/G236del/S239K/A327G, E233P/L234V/L235A/G236del/S267K/A327G and E233P/L234V/L235A/G236del, L234A/L235A/G237deleted; ii) IgG2: A330S/A331S, V234A/G237A, V234A/G237A/D265A, D265A/A330S/A331S, V234A/G237A/D265A/A330S/A331S, and H268Q/V309L/A330S/A331S; iii) IgG4: L235A/G237A/E318A, D265A, L235A/G237A/D265A and L235A/G237A/D265A/E318A.

“Epitope” as used herein refers to a determinant capable of specific binding to the variable region of an antibody molecule known as a paratope. Epitopes are groupings of molecules such as amino acids or sugar side chains and usually have specific structural characteristics, as well as specific charge characteristics. A single antigen may have more than one epitope. The epitope may comprise amino acid residues directly involved in the binding and other amino acid residues, which are not directly involved in the binding, such as amino acid residues which are effectively blocked by the antigen binding peptide (in other words, the amino acid residue is within the footprint of the antigen binding peptide). Epitopes may be either conformational or linear. An epitope typically includes at least 3, and more usually, at least 5 or 8-10 amino acids. Antibodies that recognize the same epitope can be verified in a simple immunoassay showing the ability of one antibody to block the binding of another antibody to a target antigen, for example “binning”.

“Linker” as used herein refers to a molecule that connect two polypeptide chains. Linker can be a polypeptide linker or a synthetic chemical linker (for example, see disclosed in Protein Engineering, 9(3), 299-305, 1996). The length and sequence of the polypeptide linkers is not particularly limited and can be selected according to the purpose by those skilled in the art. Polypeptide linker comprises one or more amino acids. Preferably, polypeptide linker is a peptide with a length of at least 5 amino acids, preferably with a length of 5 to 100, more preferably of 10 to 50 amino acids. In one embodiment, said peptide linker is G, S, GS, SG, SGG, GGS, and GSG (with G=glycine and S=serine). In another embodiment, said peptide linker is (GGGS)xGn (SEQ ID NO:5) or (GGGGS)xGn (SEQ ID NO:6) or (GGGGGS)xGn (SEQ ID NO:7) with x=1, 2, 3, 4, 5, 6, 7, 8, 9, 10, 11, or 12 and n=0, 1, 2 or 3. Preferably, the said linker is (GGGGS)xGn with x=2, 3, or 4 and n=0 (SEQ ID NO:8); more preferably the said linker is (GGGGS)xGn with x=3 and n=0 (SEQ ID NO:9). Synthetic chemical linkers include crosslinking agents that are routinely used to crosslink peptides, for example, N-hydroxy succinimide (NHS), disuccinimidyl suberate (DSS), bis(succinimidyl) suberate (BS3), dithiobis(succinimidyl propionate) (DSP), dithiobis(succinimidyl propionate) (DTSSP), ethylene glycol bis(succinimidyl succinate) (EGS), ethylene glycol bis(sulfosuccinimidyl succinate) (sulfo-EGS), disuccinimidyl tartrate (DST), disulfosuccinimidyl tartrate (sulfo-DST), bis[2-(succinimidoxycarbonyloxy)ethyl]sulfone (BSOCOES), and bis[2-(succinimidoxycarbonyloxy)ethyl]sulfone (sulfo-BSOCOES).

The term “polynucleotide” refers to an isolated nucleic acid molecule or construct, e.g. messenger RNA (mRNA), virally-derived RNA, or plasmid DNA (pDNA) encoding the polypeptides of the present disclosure. A polynucleotide may comprise a conventional phosphodiester bond or a non-conventional bond (e.g. an amide bond, such as found in peptide nucleic acids (PNA). The term “nucleic acid molecule” refers to any one or more nucleic acid segments, e.g. DNA or RNA fragments, present in a polynucleotide. In some aspects, one or more vectors (particularly expression vectors) comprising such nucleic acids are provided. In one aspect, a method for making a polypeptide of the present disclosure is provided, wherein the methods comprises culturing a host cell comprising a nucleic acid encoding the polypeptide under conditions suitable for expression of the polypeptide and recovering the polypeptide from the host cell. “Recombinant” means the proteins are generated using recombinant nucleic acid techniques in exogeneous host cells. Recombinantly produced proteins expressed in host cells are considered isolated for the purpose of the present disclosure, as are native or recombinant proteins which have been separated, fractionated, or partially or substantially purified by any suitable technique.

“Isolated,” when used to describe the various polypeptides disclosed herein, means a polypeptide that has been identified and separated and/or recovered from a cell or cell culture from which it was expressed. Typically, an isolated polypeptide will be purified by at least one purification step. There is no required level of purity; “purification” or “purified” refers to increase of the target protein concentration relative to the concentration of contaminants in a composition as compared to the starting material. An “isolated protein,” as used herein refers to a target protein which is substantially free of other proteins having different binding specificities.

The terms “cancer” refers the physiological condition in mammals that is typically characterized by unregulated and abnormal cell growth with the potential to invade or spread to other parts of the body. Examples of cancer include but are not limited to, carcinoma, lymphoma, blastoma, sarcoma, and leukemia. More particular examples of such cancers include lung cancer, small-cell lung cancer, non-small cell lung (NSCL) cancer, bronchioloalveolar cell lung cancer, squamous cell cancer, adenocarcinoma of the lung, squamous carcinoma of the lung, cancer of the peritoneum, head and neck cancer, bone cancer, pancreatic cancer, skin cancer, cancer of the head or neck, cutaneous or intraocular melanoma, thyroid cancer, uterine cancer, gastrointestinal cancer, ovarian cancer, rectal cancer, cancer of the anal region, stomach cancer, gastric cancer, colon cancer, breast cancer, endometrial carcinoma, uterine cancer, carcinoma of the fallopian tubes, carcinoma of the cervix, carcinoma of the vagina, vulval cancer, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, bladder cancer, liver cancer, hepatoma, hepatocellular cancer, cervical cancer, salivary gland carcinoma, biliay cancer, neoplasms of the central nervous system (CNS), spinal axis tumors, brain stem glioma, glioblastoma multiforme, astrocytomas, schwannomas, ependymomas, medulloblastomas, meningiomas, squamous cell carcinomas, pituitary adenoma and Ewings sarcoma, including refractory versions of any of the above cancers, or a combination of one or more of the above cancers.

### Mutant IL-2 Polypeptides

The present disclosure provides, inter alia, mutant IL-2 polypeptides that exhibit less than 50% of binding affinity to IL-2Rα(e.g., comprising the amino acid sequence of SEQ ID NO:2 or as shown in FIG. 1B). In some embodiments, mutant IL-2 polypeptides also exhibit less than 50% of binding affinity to IL-2Rβ (e.g., comprising the amino acid sequence of SEQ ID NO:3 or as shown in FIG. 1C). In some embodiments, mutant IL-2 polypeptides exhibit less than 50% of binding affinity to IL-2Rα and less than 50% of binding affinity to IL-2Rβ (e.g., comprising the amino acid sequence of SEQ ID NO:3 or as shown in FIG. 1C), compared to wild-type IL-2 polypeptide (e.g., comprising the amino acid sequence of SEQ ID NO:1 or as shown in FIG. 1A). In some embodiments, mutant IL-2 polypeptides exhibit less than 50% of binding affinity to IL-2Rα and less than 50% of binding affinity to IL-2Rγ (e.g., comprising the amino acid sequence of SEQ ID NO:4 or as shown in FIG. 1D), compared to wild-type IL-2 polypeptide (e.g., comprising the amino acid sequence of SEQ ID NO:1 or as shown in FIG. 1A). In some embodiments, mutant IL-2 polypeptides exhibit less than 50% of binding affinity to IL-2Rα, less than 50% of binding affinity to IL-2Rβ, and less than 50% of binding affinity to IL-2Rγ, compared to wild-type IL-2 polypeptide. Differences in binding affinity of wild-type and disclosed mutant polypeptide for IL-2Rα and IL-2Rβ can be measured, e.g., in standard surface plasmon resonance (SPR) assays that measure affinity of protein-protein interactions familiar to those skilled in the art. Differences in binding affinity of wild-type and disclosed mutant polypeptide for IL-2Rγ cannot reliably be measured by SPR assays as the affinity of wild-type IL-2 polypeptide for IL-2Rγ is very low. Instead, their reduced affinity to IL-2Rγ can be deduced by performing an in vitro assay that measures pSTAT5 and compares the activity of IL-2 polypeptides with and without the IL-2Rγ affinity-reducing substitution on IL-2R-expressing cells.

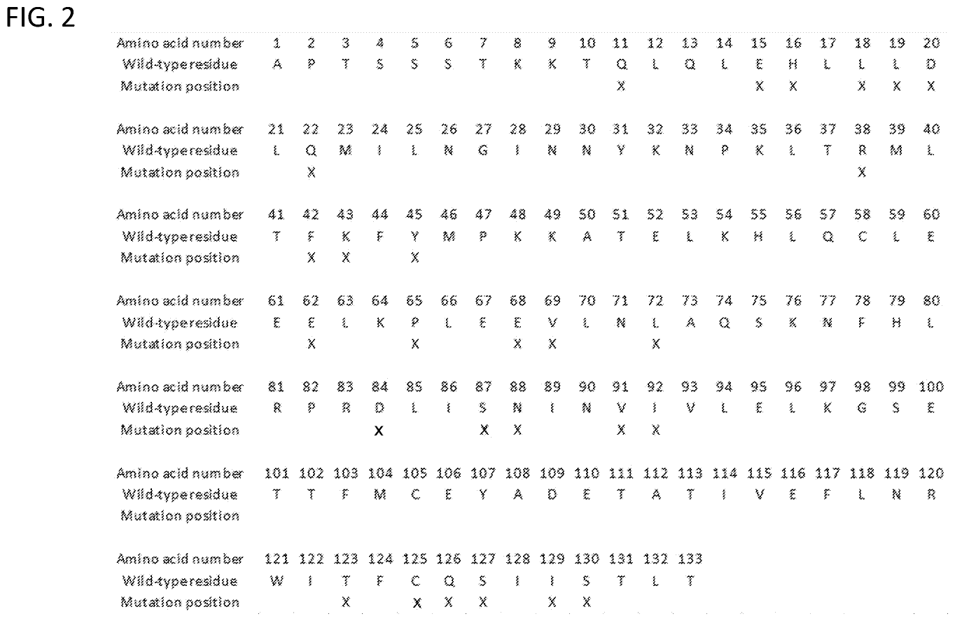

The mutant IL-2 polypeptides of the present disclosure have one or more, two or more, or three or more affinity-reducing amino acid substitutions relative to the wild-type mature IL-2 polypeptide having an amino acid sequence as depicted in FIG. 1A (SEQ ID NO:1), wherein one or more, two or more, or three or more substituted residues, are selected from the following group: Q11, H16, L18, L19, D20, D84, S87, Q22, R38, F42, K43, Y45, E62, P65, E68, V69, L72, D84, S87, N88, V91, 192, T123, Q126, S127, I129, and S130. The location of possible amino acid substitutions in the sequence of the wild-type mature IL-2 polypeptide is depicted, e.g., in FIG. 2. Decreased affinity to IL-2Rα may be obtained by substituting one or more of the following residues in the sequence of the wild-type mature IL-2 polypeptide: R38, F42, K43, Y45, E62, P65, E68, V69, and L72. Decreased affinity to IL-2Rβ may be obtained by substituting one or more of the following residues: E15, H16, L19, D20, D84, S87, N88, V91, and 192. Decreased affinity to IL-2Rγ may be obtained by substituting one or more of the following residues in the sequence of the wild-type mature IL-2 polypeptide: Q11, L18, Q22, T123, Q126, S127, I129, and S130.

In some embodiments, the mutant IL-2 polypeptide comprises an F42A or F42K amino acid substitution relative to the wild-type mature IL-2 amino acid sequence, e.g., as depicted in FIG. 1A (SEQ ID NO:1). In some embodiments, the mutant IL-2 polypeptide comprises an F42A or F42K amino acid substitution and an R38A, R38D, R38E, E62Q, E68A, E68Q, E68K, or E68R amino acid substitution relative to the wild-type mature IL-2 amino acid sequence, e.g., as depicted in FIG. 1A (SEQ ID NO:1). For example, in some embodiments, the mutant IL-2 polypeptide comprises F42A; R38A and F42A; R38D and F42A; R38E and F42A; F42A and E62Q; F42A and E68A; F42A and E68Q; F42A and E68K; F42A and E68R; or R38A and F42K amino acid substitution(s) relative to the wild-type mature IL-2 amino acid sequence, e.g., as depicted in FIG. 1A (SEQ ID NO:1). In some embodiments, the mutant IL-2 polypeptide comprises R38E and F42A amino acid substitutions relative to the wild-type IL-2 amino acid sequence. In some embodiments, the mutant IL-2 polypeptide comprises R38D and F42A amino acid substitutions relative to the wild-type IL-2 amino acid sequence. In some embodiments, the mutant IL-2 polypeptide comprises F42A and E62Q amino acid substitutions relative to the wild-type IL-2 amino acid sequence. In some embodiments, the mutant IL-2 polypeptide comprises R38A and F42K amino acid substitutions relative to the wild-type IL-2 amino acid sequence. In some embodiments, the mutant IL-2 polypeptide comprises R38D and F42A amino acid substitutions relative to the wild-type IL-2 amino acid sequence. In some embodiments, the mutant IL-2 polypeptide comprises R38A and F42K amino acid substitutions relative to the wild-type IL-2 amino acid sequence. In some embodiments, the mutant IL-2 polypeptide comprises F42A and E62Q amino acid substitutions relative to the wild-type IL-2 amino acid sequence. In some embodiments, the mutant IL-2 polypeptide comprises an H16E, H16D, D20N, M23A, M23R, M23K, D84L, D84N, D84V, D84H, D84Y, D84R, D84K, S87K, S87A, N88A, N88S, N88T, N88R, N88I, V91A, V91T, V91E, I92A, E95S, E95A, E95R, T123A, T123E, T123K, T123Q, Q126A, Q126S, Q126T, Q126E, S127A, S127E, S127K, or S127Q amino acid substitution relative to the wild-type IL-2 amino acid sequence. In some embodiments, the mutant IL-2 polypeptide comprises F42A; R38A and F42A; R38D and F42A; R38E and F42A; F42A and E62Q; F42A and E68A; F42A and E68Q; F42A and E68K; F42A and E68R; or R38A and F42K amino acid substitution(s) relative to the wild-type mature IL-2 amino acid sequence depicted in FIG. 1A and an H16E, H16D, D20N, M23A, M23R, M23K, D84L, D84N, D84V, D84H, D84Y, D84R, D84K, S87K, S87A, N88A, N88S, N88T, N88R, N88I, V91A, V91T, V91E, I92A, E95S, E95A, E95R, T123A, T123E, T123K, T123Q, Q126A, Q126S, Q126T, Q126E, S127A, S127E, S127K, or S127Q amino acid substitution relative to the wild-type mature IL-2 amino acid sequence depicted in FIG. 1A (SEQ ID NO:1). For example, in some embodiments, the mutant IL-2 polypeptide comprises R38E, F42A, and H16E amino acid substitutions relative to the wild-type IL-2 amino acid sequence. In some embodiments, the mutant IL-2 polypeptide comprises R38E, F42A, and H16D amino acid substitutions relative to the wild-type IL-2 amino acid sequence. In some embodiments, the mutant IL-2 polypeptide comprises R38E, F42A, and N88S amino acid substitutions relative to the wild-type IL-2 amino acid sequence. In some embodiments, the mutant IL-2 polypeptide comprises R38E, F42A, and N88A amino acid substitutions relative to the wild-type IL-2 amino acid sequence. In some embodiments, the mutant IL-2 polypeptide comprises R38E, F42A, and V91E amino acid substitutions relative to the wild-type IL-2 amino acid sequence. In some embodiments, the mutant IL-2 polypeptide comprises R38E, F42A, and Q126S amino acid substitutions relative to the wild-type IL-2 amino acid sequence. In some embodiments, the mutant IL-2 polypeptide comprises the amino acid sequence of SEQ ID NO:1 with one of the following sets of amino acid substitutions (relative to the sequence of SEQ ID NO:1): R38E and F42A; R38D and F42A; F42A and E62Q; R38A and F42K; R38E, F42A, and N88S; R38E, F42A, and N88A; R38E, F42A, and V91E; R38E, F42A, and D84H; H16D, R38E and F42A; H16E, R38E and F42A; R38E, F42A and Q126S; R38D, F42A and N88S; R38D, F42A and N88A; R38D, F42A and V91E; R38D, F42A, and D84H; H16D, R38D and F42A; H16E, R38D and F42A; R38D, F42A and Q126S; R38A, F42K, and N88S; R38A, F42K, and N88A; R38A, F42K, and V91E; R38A, F42K, and D84H; H16D, R38A, and F42K; H16E, R38A, and F42K; R38A, F42K, and Q126S; F42A, E62Q, and N88S; F42A, E62Q, and N88A; F42A, E62Q, and V91E; F42A, E62Q, and D84H; H16D, F42A, and E62Q; H16E, F42A, and E62Q; F42A, E62Q, and Q126S; R38E, F42A, and C125A; R38D, F42A, and C125A; F42A, E62Q, and C125A; R38A, F42K, and C125A; R38E, F42A, N88S, and C125A; R38E, F42A, N88A, and C125A; R38E, F42A, V91E, and C125A; R38E, F42A, D84H, and C125A; H16D, R38E, F42A, and C125A; H16E, R38E, F42A, and C125A; R38E, F42A, C125A and Q126S; R38D, F42A, N88S, and C125A; R38D, F42A, N88A, and C125A; R38D, F42A, V91E, and C125A; R38D, F42A, D84H, and C125A; H16D, R38D, F42A, and C125A; H16E, R38D, F42A, and C125A; R38D, F42A, C125A, and Q126S; R38A, F42K, N88S, and C125A; R38A, F42K, N88A, and C125A; R38A, F42K, V91E, and C125A; R38A, F42K, D84H, and C125A; H16D, R38A, F42K, and C125A; H16E, R38A, F42K, and C125A; R38A, F42K, C125A and Q126S; F42A, E62Q, N88S, and C125A; F42A, E62Q, N88A, and C125A; F42A, E62Q, V91E, and C125A; F42A, E62Q, and D84H, and C125A; H16D, F42A, and E62Q, and C125A; H16E, F42A, E62Q, and C125A; F42A, E62Q, C125A and Q126S; F42A, N88S, and C125A; F42A, N88A, and C125A; F42A, V91E, and C125A; F42A, D84H, and C125A; H16D, F42A, and C125A; H16E, F42A, and C125A; and F42A, C125A and Q126S. In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRDLISNINVIVLELKGSETTFMCEYADETATIVEFLNR WITFCQSIISTLT (SEQ ID NO:18). In some embodiments, the mutant IL-2 polypeptide comprises the amino acid sequence of APTSSSTKKTQLQLEHLLLDLQMILNGINNYKNPKLTDMLTAKFYMPKKATELKHILQCL EEELKPLEEVLNLAQSKNFHLRPRDLISNINVIVLELKGSETTFMCEYADETATIVEFLNR WITFCQSIISTLT (SEQ ID NO:19). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRDLISNINVIVLELKGSETTFMCEYADETATIVEFLNR WITFCQSIISTLT (SEQ ID NO:20). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRDLISNINVIVLELKGSETTFMCEYADETATIVEFLNR WITFCQSIISTLT (SEQ ID NO:21). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRDLISSINVIVLELKGSETTFMCEYADETATIVEFLNR WITFCQSIISTLT (SEQ ID NO:22). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRDLISAINVIVLELKGSETTFMCEYADETATIVEFLNR WITFCQSIISTLT (SEQ ID NO:23). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRDLISNINEIVLELKGSETTFMCEYADETATIVEFLNR WITFCQSIISTLT (SEQ ID NO:24). In some embodiments, the mutant IL-2 polypeptide comprises the amino acid sequence of APTSSSTKKTQLQLEHLLLDLQMILNGINNYKNPKLTEMLTAKFYMPKKATELKHILQCL EEELKPLEEVLNLAQSKNFHILRPRHLISNINVIVLELKGSETTFMCEYADETATIVEFLNR WITFCQSIISTLT (SEQ ID NO:25). In some embodiments, the mutant IL-2 polypeptide comprises the amino acid sequence of APTSSSTKKTQLQLEDLLLDLQMILNGINNYKNPKLTEMLTAKFYMPKKATELKHILQCL EEELKPLEEVLNLAQSKNFHLRPRDLISNINVIVLELKGSETTFMCEYADETATIVEFLNR WITFCQSIISTLT (SEQ ID NO:26). In some embodiments, the mutant IL-2 polypeptide comprises the amino acid sequence of APTSSSTKKTQLQLEELLLDLQMILNGINNYKNPKLTEMLTAKFYMPKKATELKHILQCL EEELKPLEEVLNLAQSKNFHLRPRDLISNINVIVLELKGSETTFMCEYADETATIVEFLNR WITFCQSIISTLT (SEQ ID NO:27). In some embodiments, the mutant IL-2 polypeptide comprises the amino acid sequence of APTSSSTKKTQLQLEHLLLDLQMILNGINNYKNPKLTEMLTAKFYMPKKATELKHILQCL EEELKPLEEVLNLAQSKNFHLRPRDLISNINVIVLELKGSETTFMCEYADETATIVEFLNR WITFCSSIISTLT (SEQ ID NO:28). In some embodiments, the mutant IL-2 polypeptide comprises the amino acid sequence of APTSSSTKKTQLQLEHLLLDLQMILNGINNYKNPKLTDMLTAKFYMPKKATELKHILQCL EEELKPLEEVLNLAQSKNFHLRPRDLISSINVIVLELKGSETTFMCEYADETATIVEFLNR WITFCQSIISTLT (SEQ ID NO:29). In some embodiments, the mutant IL-2 polypeptide comprises the amino acid sequence of APTSSSTKKTQLQLEHLLLDLQMILNGINNYKNPKLTDMLTAKFYMPKKATELKHILQCL EEELKPLEEVLNLAQSKNFHLRPRDLISAINVIVLELKGSETTFMCEYADETATIVEFLNR WITFCQSIISTLT (SEQ ID NO:30). In some embodiments, the mutant IL-2 polypeptide comprises the amino acid sequence of APTSSSTKKTQLQLEHLLLDLQMILNGINNYKNPKLTDMLTAKFYMPKKATELKHILQCL EEELKPLEEVLNLAQSKNFHLRPRDLISNINEIVLELKGSETTFMCEYADETATIVEFLNR WITFCQSIISTLT (SEQ ID NO:31). In some embodiments, the mutant IL-2 polypeptide comprises the amino acid sequence of APTSSSTKKTQLQLEHLLLDLQMILNGINNYKNPKLTDMLTAKFYMPKKATELKHILQCL EEELKPLEEVLNLAQSKNFHLRPRHLISNINVIVLELKGSETTFMCEYADETATIVEFLNR WITFCQSIISTLT (SEQ ID NO:32). In some embodiments, the mutant IL-2 polypeptide comprises the amino acid sequence of APTSSSTKKTQLQLEDLLLDLQMILNGINNYKNPKLTDMLTAKFYMPKKATELKHILQCL EEELKPLEEVLNLAQSKNFHLRPRDLISNINVIVLELKGSETTFMCEYADETATIVEFLNR WITFCQSIISTLT (SEQ ID NO:33). In some embodiments, the mutant IL-2 polypeptide comprises the amino acid sequence of APTSSSTKKTQLQLEELLLDLQMILNGINNYKNPKLTDMLTAKFYMPKKATELKHLQCL EEELKPLEEVLNLAQSKNFHLRPRDLISNINVIVLELKGSETTFMCEYADETATIVEFLNR WITFCQSIISTLT (SEQ ID NO:34). In some embodiments, the mutant IL-2 polypeptide comprises the amino acid sequence of APTSSSTKKTQLQLEHLLLDLQMILNGINNYKNPKLTDMLTAKFYMPKKATELKHLQCL EEELKPLEEVLNLAQSKNFHLRPRDLISNINVIVLELKGSETTFMCEYADETATIVEFLNR WITFCSSIISTLT (SEQ ID NO:35). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRDLISSINVIVLELKGSETTFMCEYADETATIVEFLNR WITFCQSIISTLT (SEQ ID NO:36). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRDLISAINVIVLELKGSETTFMCEYADETATIVEFLNR WITFCQSIISTLT (SEQ ID NO:37). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRDLISNINEIVLELKGSETTFMCEYADETATIVEFLNR WITFCQSIISTLT (SEQ ID NO:38). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRHLISNINVIVLELKGSETTFMCEYADETATIVEFLNR WITFCQSIISTLT (SEQ ID NO:39). In some embodiments, the mutant IL-2 polypeptide comprises the amino acid sequence of APTSSSTKKTQLQLEDLLLDLQMILNGINNYKNPKLTAMLTKKFYMPKKATELKHLQCL EEELKPLEEVLNLAQSKNFHLRPRDLISNINVIVLELKGSETTFMCEYADETATIVEFLNR WITFCQSIISTLT (SEQ ID NO:40). In some embodiments, the mutant IL-2 polypeptide comprises the amino acid sequence of APTSSSTKKTQLQLEELLLDLQMILNGINNYKNPKLTAMLTKKFYMPKKATELKHLQCL EEELKPLEEVLNLAQSKNFHLRPRDLISNINVIVLELKGSETTFMCEYADETATIVEFLNR WITFCQSIISTLT (SEQ ID NO:41). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRDLISNINVIVLELKGSETTFMCEYADETATIVEFLNR WITFCSSIISTLT (SEQ ID NO:42). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRDLISSINVIVLELKGSETTFMCEYADETATIVEFLNR WITFCQSIISTLT (SEQ ID NO:43). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRDLISAINVIVLELKGSETTFMCEYADETATIVEFLNR WITFCQSIISTLT (SEQ ID NO:44). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRDLISNINEIVLELKGSETTFMCEYADETATIVEFLNR WITFCQSIISTLT (SEQ ID NO:45). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRHLISNINVIVLELKGSETTFMCEYADETATIVEFLNR WITFCQSIISTLT (SEQ ID NO:46). In some embodiments, the mutant IL-2 polypeptide comprises the amino acid sequence of APTSSSTKKTQLQLEDLLLDLQMILNGINNYKNPKLTRMLTAKFYMPKKATELKHLQCL EEQLKPLEEVLNLAQSKNFHLRPRDLISNINVIVLELKGSETTFMCEYADETATIVEFLNR WITFCQSIISTLT (SEQ ID NO:47). In some embodiments, the mutant IL-2 polypeptide comprises the amino acid sequence of APTSSSTKKTQLQLEELLLDLQMILNGINNYKNPKLTRMLTAKFYMPKKATELKHLQCL EEQLKPLEEVLNLAQSKNFHLRPRDLISNINVIVLELKGSETTFMCEYADETATIVEFLNR WITFCQSIISTLT (SEQ ID NO:48). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRDLISNINVIVLELKGSETTFMCEYADETATIVEFLNR WITFCSSIISTLT (SEQ ID NO:49). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRDLISNINVIVLELKGSETTFMCEYADETATIVEFLNR WITFAQSIISTLT (SEQ ID NO: 50). In some embodiments, the mutant IL-2 polypeptide comprises the amino acid sequence of APTSSSTKKTQLQLEHLLLDLQMILNGINNYKNPKLTDMLTAKFYMPKKATELKHLQCL EEELKPLEEVLNLAQSKNFHLRPRDLISNINVIVLELKGSETTFMCEYADETATIVEFLNR WITFAQSIISTLT (SEQ ID NO:51). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRDLISNINVIVLELKGSETTFMCEYADETATIVEFLNR WITFAQSIISTLT (SEQ ID NO: 52). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRDLISNINVIVLELKGSETTFMCEYADETATIVEFLNR WITFAQSIISTLT (SEQ ID NO:53). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRDLISSINVIVLELKGSETTFMCEYADETATIVEFLNR WITFAQSIISTLT (SEQ ID NO: 54). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRDLISAINVIVLELKGSETTFMCEYADETATIVEFLNR WITFAQSIISTLT (SEQ ID NO:55). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRDLISNINEIVLELKGSETTFMCEYADETATIVEFLNR WITFAQSIISTLT (SEQ ID NO: 56). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRHLISNINVIVLELKGSETTFMCEYADETATIVEFLNR WITFAQSIISTLT (SEQ ID NO: 57). In some embodiments, the mutant IL-2 polypeptide comprises the amino acid sequence of APTSSSTKKTQLQLEDLLLDLQMILNGINNYKNPKLTEMLTAKFYMPKKATELKHLQCL EEELKPLEEVLNLAQSKNFHLRPRDLISNINVIVLELKGSETTFMCEYADETATIVEFLNR WITFAQSIISTLT (SEQ ID NO:58). In some embodiments, the mutant IL-2 polypeptide comprises the amino acid sequence of APTSSSTKKTQLQLEELLLDLQMILNGINNYKNPKLTEMLTAKFYMPKKATELKHLQCL EEELKPLEEVLNLAQSKNFHLRPRDLISNINVIVLELKGSETTFMCEYADETATIVEFLNR WITFAQSIISTLT (SEQ ID NO: 59). In some embodiments, the mutant IL-2 polypeptide comprises the amino acid sequence of APTSSSTKKTQLQLEHLLLDLQMILNGINNYKNPKLTEMLTAKFYMPKKATELKHLQCL EEELKPLEEVLNLAQSKNFHLRPRDLISNINVIVLELKGSETTFMCEYADETATIVEFLNR WITFASSIISTLT (SEQ ID NO:60). In some embodiments, the mutant IL-2 polypeptide comprises the amino acid sequence of APTSSSTKKTQLQLEHLLLDLQMILNGINNYKNPKLTDMLTAKFYMPKKATELKHLQCL EEELKPLEEVLNLAQSKNFHLRPRDLISNINVIVLELKGSETTFMCEYADETATIVEFLNR WITFAQSIISTLT (SEQ ID NO:61). In some embodiments, the mutant IL-2 polypeptide comprises the amino acid sequence of APTSSSTKKTQLQLEHLLLDLQMILNGINNYKNPKLTDMLTAKFYMPKKATELKHLQCL EEELKPLEEVLNLAQSKNFHLRPRDLISSINVIVLELKGSETTFMCEYADETATIVEFLNR WITFAQSIISTLT (SEQ ID NO:62). In some embodiments, the mutant IL-2 polypeptide comprises the amino acid sequence of APTSSSTKKTQLQLEHLLLDLQMILNGINNYKNPKLTDMLTAKFYMPKKATELKHLQCL EEELKPLEEVLNLAQSKNFHLRPRDLISNINEIVLELKGSETTFMCEYADETATIVEFLNR WITFAQSIISTLT (SEQ ID NO:63). In some embodiments, the mutant IL-2 polypeptide comprises the amino acid sequence of APTSSSTKKTQLQLEHLLLDLQMILNGINNYKNPKLTDMLTAKFYMPKKATELKHLQCL EEELKPLEEVLNLAQSKNFHLRPRHLISNINVIVLELKGSETTFMCEYADETATIVEFLNR WITFAQSIISTLT (SEQ ID NO:64). In some embodiments, the mutant IL-2 polypeptide comprises the amino acid sequence of APTSSSTKKTQLQLEDLLLDLQMILNGINNYKNPKLTDMLTAKFYMPKKATELKHLQCL EEELKPLEEVLNLAQSKNFHLRPRDLISNINVIVLELKGSETTFMCEYADETATIVEFLNR WITFAQSIISTLT (SEQ ID NO:65). In some embodiments, the mutant IL-2 polypeptide comprises the amino acid sequence of APTSSSTKKTQLQLEELLLDLQMILNGINNYKNPKLTDMLTAKFYMPKKATELKHLQCL EEELKPLEEVLNLAQSKNFHLRPRDLISNINVIVLELKGSETTFMCEYADETATIVEFLNR WITFAQSIISTLT (SEQ ID NO:66). In some embodiments, the mutant IL-2 polypeptide comprises the amino acid sequence of APTSSSTKKTQLQLEHLLLDLQMILNGINNYKNPKLTDMLTAKFYMPKKATELKHLQCL EEELKPLEEVLNLAQSKNFHLRPRDLISNINVIVLELKGSETTFMCEYADETATIVEFLNR WITFASSIISTLT (SEQ ID NO:67). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRDLISNINVIVLELKGSETTFMCEYADETATIVEFLNR WITFAQSIISTLT (SEQ ID NO:68). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRDLISAINVIVLELKGSETTFMCEYADETATIVEFLNR WITFAQSIISTLT (SEQ ID NO:69). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRDLISNINEIVLELKGSETTFMCEYADETATIVEFLNR WITFAQSIISTLT (SEQ ID NO:70). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRHLISNINVIVLELKGSETTFMCEYADETATIVEFLNR WITFAQSIISTLT (SEQ ID NO:71). In some embodiments, the mutant IL-2 polypeptide comprises the amino acid sequence of APTSSSTKKTQLQLEDLLLDLQMILNGINNYKNPKLTAMLTKKFYMPKKATELKHLQCL EEELKPLEEVLNLAQSKNFHLRPRDLISNINVIVLELKGSETTFMCEYADETATIVEFLNR WITFAQSIISTLT (SEQ ID NO:72). In some embodiments, the mutant IL-2 polypeptide comprises the amino acid sequence of APTSSSTKKTQLQLEELLLDLQMILNGINNYKNPKLTAMLTKKFYMPKKATELKHLQCL EEELKPLEEVLNLAQSKNFHLRPRDLISNINVIVLELKGSETTFMCEYADETATIVEFLNR WITFAQSIISTLT (SEQ ID NO:73). In some embodiments, the mutant IL-2 polypeptide comprises the amino acid sequence of APTSSSTKKTQLQLEHLLLDLQMILNGINNYKNPKLTAMLTKKFYMPKKATELKHLQCL EEELKPLEEVLNLAQSKNFHLRPRDLISNINVIVLELKGSETTFMCEYADETATIVEFLNR WITFASSIISTLT (SEQ ID NO:74). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRDLISNINVIVLELKGSETTFMCEYADETATIVEFLNR WITFAQSIISTLT (SEQ ID NO:75). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRDLISAINVIVLELKGSETTFMCEYADETATIVEFLNR WITFAQSIISTLT (SEQ ID NO:76). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRDLISNINEIVLELKGSETTFMCEYADETATIVEFLNR WITFAQSIISTLT (SEQ ID NO:77). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRHLISNINVIVLELKGSETTFMCEYADETATIVEFLNR WITFAQSIISTLT (SEQ ID NO:78). In some embodiments, the mutant IL-2 polypeptide comprises the amino acid sequence of APTSSSTKKTQLQLEDLLLDLQMILNGINNYKNPKLTRMLTAKFYMPKKATELKHLQCL EEQLKPLEEVLNLAQSKNFHLRPRDLISNINVIVLELKGSETTFMCEYADETATIVEFLNR WITFAQSIISTLT (SEQ ID NO:79). In some embodiments, the mutant IL-2 polypeptide comprises the amino acid sequence of APTSSSTKKTQLQLEELLLDLQMILNGINNYKNPKLTRMLTAKFYMPKKATELKHLQCL EEQLKPLEEVLNLAQSKNFHLRPRDLISNINVIVLELKGSETTFMCEYADETATIVEFLNR WITFAQSIISTLT (SEQ ID NO: 80). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEQLKPLEEVLNLAQSKNFHLRPRDLISNINVIVLELKGSETTFMCEYADETATIVEFLNR WITFASSIISTLT (SEQ ID NO:81). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEELKPLEEVLNLAQSKNFHLRPRDLISNINVIVLELKGSETTFMCEYADETATIVEFLNR WITFAQSIISTLT (SEQ ID NO:82). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEELKPLEEVLNLAQSKNFHLRPRDLISSINVIVLELKGSETTFMCEYADETATIVEFLNR WITFAQSIISTLT (SEQ ID NO:83). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEELKPLEEVLNLAQSKNFHLRPRDLISNINEIVLELKGSETTFMCEYADETATIVEFLNR WITFAQSIISTLT (SEQ ID NO:84). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEELKPLEEVLNLAQSKNFHLRPRHLISNINVIVLELKGSETTFMCEYADETATIVEFLNR WITFAQSIISTLT (SEQ ID NO:85). In some embodiments, the mutant IL-2 polypeptide comprises the amino acid sequence of APTSSSTKKTQLQLEDLLLDLQMILNGINNYKNPKLTRMLTAKFYMPKKATELKHLQCL EEELKPLEEVLNLAQSKNFHLRPRDLISNINVIVLELKGSETTFMCEYADETATIVEFLNR WITFAQSIISTLT (SEQ ID NO:86). In some embodiments, the mutant IL-2 polypeptide comprises the amino acid sequence of APTSSSTKKTQLQLEELLLDLQMILNGINNYKNPKLTRMLTAKFYMPKKATELKHLQCL EEELKPLEEVLNLAQSKNFHLRPRDLISNINVIVLELKGSETTFMCEYADETATIVEFLNR WITFAQSIISTLT (SEQ ID NO:87). In some embodiments, the mutant IL-2 polypeptide comprises the amino acid sequence of APTSSSTKKTQLQLEHLLLDLQMILNGINNYKNPKLTRMLTAKFYMPKKATELKHLQCL EEELKPLEEVLNLAQSKNFHLRPRDLISNINVIVLELKGSETTFMCEYADETATIVEFLNR WITFASSIISTLT (SEQ ID NO:88).

In some embodiments, the mutant IL-2 polypeptides of the present disclosure also contain other modifications, including but not limited to mutations and deletions, that provide additional advantages such as improved biophysical properties. Improved biophysical properties include but are not limited to improved thermostability, aggregation propensity, acid reversibility, viscosity, and production in a mammalian or bacterial or yeast cell. For example, residue C125 may be replaced with a neutral amino acid such as serine, alanine, threonine or valine; and N terminal A1 residue could be deleted, both of which were described in U.S. Pat. No. 4,518,584. Mutant IL-2 polypeptides may also include a mutation of the residue M104, such as M104A, as described in U.S. Pat. No. 5,206,344. Thus, in certain embodiments the mutant IL-2 polypeptide of the present disclosure comprises the amino acid substitution C125A. In other embodiments, one, two, or three N-terminal residues are deleted.

### Fusion Proteins

The present disclosure provides fusion proteins comprising the mutant IL-2 polypeptides of the present disclosure and antigen binding molecules binding to one of the following antigens: CD8α, CD8β, and PD1 wherein said fusion proteins preferentially activate immune cells expressing the antigen for the antigen binding molecule of the fusion over immune cells not expressing said antigen.

Preferential activity of the targeted IL-2 fusion proteins comprising the mutant IL-2 polypeptides on antigen-expressing cells is demonstrated in assays that contain antigen-expressing and antigen-non expressing cells that also express IL-2Rβγ or IL-2Rαβγ. One such assay is an in vitro assay that measures STAT5 (pSTAT5) phosphorylation and/or expression of the proliferation marker Ki-67 in human immune cells, such as human peripheral blood and/or tumor-infiltrating immune cells upon exposure to IL-2 polypeptides. In one format of the assay, the activity of the targeted IL-2 fusion protein is measured on antigen-expressing and non-expressing cells to demonstrate the selectivity on antigen-expressing cells. In another format of the assay, the activity of the targeted IL-2 fusion protein comprising the mutant IL-2 polypeptide on antigen-expressing cells is compared to that of the untargeted IL-2 fusion protein comprising the same mutant IL-2 polypeptide and a control antibody not recognizing any antigens on antigen-expressing cells to demonstrate the magnitude of rescue in signaling of the mutant IL-2 polypeptide when fused to an antigen binding molecule.

In some embodiments, the fusion protein of the present disclosure containing CD8α antigen binding molecules activates CD8α+IL-2Rβ+ cells over CD8α−IL-2Rβ+ cells, by at least 10-fold, at least 50-fold, or at least 100-fold. In some embodiments, said fusion protein activate CD8α+IL-2Rβ+ cells more than 50-fold, 100 fold, or 200 fold compared to a fusion molecule comprising the said IL-2 mutant polypeptide and a control antibody not binding to any antigens expressed on said cells. Said cell activation by the IL-2 fusion protein is determined by measuring the expression of pSTAT5 or the cell proliferation marker Ki67 in said cells following the treatment with said IL-2 fusion protein.

In some embodiments, the fusion protein of the present disclosure containing CD8β antigen binding molecules activates CD8β+IL-2Rβ+ cells over CD8β− IL-2Rβ+ cells by at least 10-fold, at least 50-fold, or at least 100-fold. In some embodiments, said fusion protein activates CD8β+IL-2Rβ+ cells more than 50-fold, 100 fold, or 200 fold compared to a fusion molecule comprising the said IL-2 mutant polypeptide and a control antibody not binding to any antigens expressed on said cells. Said cell activation by the IL-2 fusion protein is determined by measuring the expression of pSTAT5 or the cell proliferation marker Ki67 in said cells following the treatment with said IL-2 fusion protein

In some embodiments, fusion protein of the present disclosure containing PD1 antigen binding molecules activates PD1+IL-2Rβ+ cells over PD1−IL-2Rβ+ cells by at least 10-fold, at least 50-fold, or at least 100-fold. In some embodiments, said fusion protein activates PD1+IL-2Rβ+ cells more than 50-fold, 100 fold, or 200 fold compared to a fusion protein comprising the said IL-2 mutant polypeptide and a control antibody not binding to any antigens expressed on said cells. Said cell activation by the IL-2 fusion protein is determined by measuring the expression of pSTAT5 or the cell proliferation marker Ki67 in said cells following the treatment with said IL-2 fusion protein.

In some embodiments, a fusion protein of the present disclosure displays one or more of the following: binds human CD8 and does not block an interaction of CD8 with MHC class I; and activates CD8+ T cells with at least 10-fold, 25-fold, 50-fold, 100-fold, 250-fold, 500-fold, or 1000-fold greater potency, e.g., as compared to activation of NK cells. In some embodiments, whether an anti-CD8 antibody or fusion protein of the present disclosure blocks the interaction of CD8 with MHC class I can be assayed, e.g., by assaying activation status of CD8+ T cells (e.g., upon antigen stimulation) in the presence or absence of the anti-CD8 antibody or fusion protein. For an exemplary assay and conditions, see, e.g., Example 3. In some embodiments, activation of CD8+ T cells and/or NK cells can be measured, e.g., by assaying one or more markers (e.g., proportion of treated cells expressing one or more markers) of proliferation (e.g., Ki67), IL-2Rβ/γ downstream signaling, and/or STAT5 downstream signaling. For an exemplary assay and conditions, see, e.g., Example 5.

By extension of these findings, the fusion proteins of the disclosure may contain polypeptides that bind to IL-2Rαβγ for which reduction in binding affinity to IL2Rα was achieved by methods other than introducing a small number of mutations in the sequence of the wild-type IL-2 polypeptide. Therefore, the fusion proteins of the invention may include IL-2 polypeptides that were fused to IL-2Rα as described in Lopes et al, J Immunother Cancer. 2020; 8 (1): e000673; or synthetic polypeptide mimics computationally designed to bind to IL-2Rβγ, but not to IL-2Rα, such as the one described in Silva et al, Nature. 2019 January; 565(7738):186-191; or by using an antigen binding domain polypeptide that is agonistic to IL-2Rγγ. Such polypeptides can be fused to CD8 antibodies to construct fusions resulting in their selective potency for CD8+ T cells compared to NK cells of 10 fold or more.

### Fusion Protein Formats

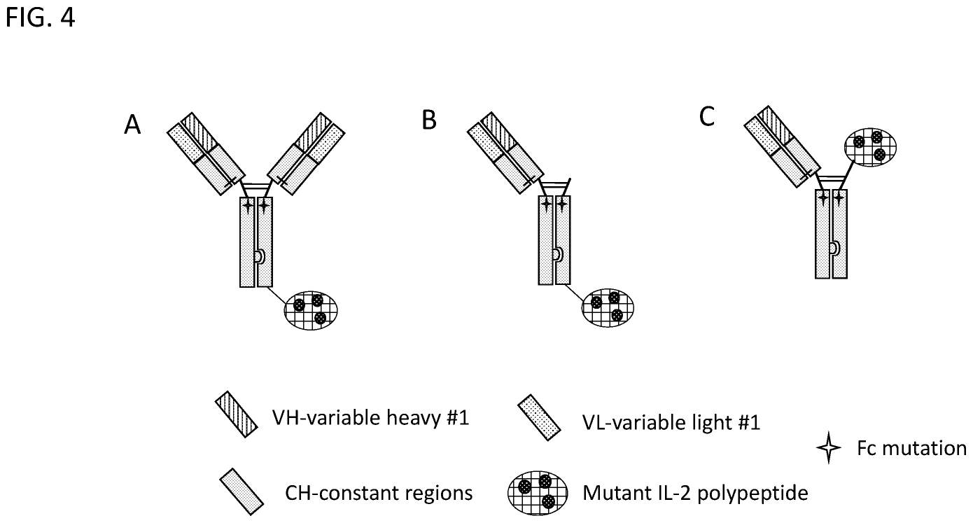

Said fusion proteins have different formats as depicted in FIG. 4. In some embodiments, the fusion protein comprises two moieties as depicted in FIG. 4A wherein: i) the first moiety is a polypeptide comprising an antibody heavy chain VH—CH1-hinge-CH2-CH3 monomer wherein VH is a variable heavy chain and CH2-CH3 is a Fc domain, an antibody light chain VL-CL wherein VL is a variable light chain and CL is a constant light chain, and the mutant IL-2 polypeptide, wherein the N-terminus of the mutant IL-2 polypeptide is fused to the C-terminus of the Fc domain via a linker; ii) the second moiety is a polypeptide comprising an antibody heavy chain VH—CH1-hinge-CH2-CH3 monomer and an antibody light chain VL-CL; and wherein, both the first and second moiety bind to an epitope on one antigen selected from the following group: human CD8α, human CD8β, and human PD1.

In some embodiments, the fusion protein comprises two moieties as depicted in FIG. 4B wherein: i) the first moiety is a polypeptide comprising an antibody hinge-CH2-CH3 monomer wherein CH2-CH3 is a Fc domain, and the mutant IL-2 polypeptide, wherein the N-terminus of the mutant IL-2 polypeptide is fused to the C-terminus end of the Fc domain via a linker; ii) the second moiety is a polypeptide comprising an antibody heavy chain VH—CH1-hinge-CH2-CH3 monomer and an antibody light chain VL-CL; and wherein the second moiety binds to an epitope on one antigen selected from the following group: human CD8α, human CD8β, and human PD1.

In some embodiments, the fusion protein comprises two moieties as depicted in FIG. 4C wherein: i) the first moiety is a polypeptide comprising an antibody hinge-CH2-CH3 monomer wherein CH2-CH3 is a Fc domain, and the mutant IL-2 polypeptide, wherein the C-terminus of the mutant IL-2 polypeptide is fused to the N-terminus end of the Fc domain via a linker; ii) the second moiety is a polypeptide comprising an antibody heavy chain VH—CH1-hinge-CH2-CH3 monomer and an antibody light chain VL-CL; and wherein the second moiety binds to an epitope on one antigen selected from the following group: human CD8α, human CD8β, and human PD1.

In some embodiments, said first and second Fc domains of the fusion protein contain the following Fc mutations to decrease effector function according to EU numbering: L234A, L235A, G237A, and K322A. In some embodiments, said first and second Fc domains of the fusion protein contain the following Fc mutations to decrease effector function according to EU numbering: L234A, L235A, G237A, and K322A. In some embodiments, said first and second Fc domains of the fusion protein contain the following amino acid substitutions to facilitate heterodimeric formation: Y349C/T366W (knob) and S354C, T366S, L368A and Y407V (hole).

In some embodiments, the recombinant bispecific antibodies and/or fusion proteins disclosed herein can be very roughly classified in two categories, namely i) formats resulting from the combination of variable regions only and ii) formats combining variable regions with Fc domains. Representatives of the first category are tandem scFv (taFv), diabodies (db), DART, single-chain diabodies (scDbs), Fab-Fc, tandem Fab, Dual variable region Fab and tandem dAb/VHH. The two variable regions can be linked together via covalent bonds or non-covalent interaction.

In some embodiments, bispecific antibodies/fusion proteins are generated on the natural immunoglobulin architecture containing two pairs of heavy chain and light chain combination with each pair having distinct binding specificity. Homodimerization of the two heavy chains in an IgG is mediated by the CH3 interaction. To promote heterodimeric formation, genetic modifications are introduced to the two respective CH3 regions. There heterodimerization mutations often involve steric repulsion, charge steering interaction, or interchain disulfide bond formation. Exemplary Fc modifications to promote heterodimerization include, without limitation, the following:

TABLE A
Fc modifications (EU numbering) that promote heterodimerization.

| Strategy | CH3 domain 1 | CH3 domain 2 | References |
|---|---|---|---|
| knobs-into-holes 1 | T366Y | Y407T | Brinkmann & Kontermann, |
| knobs-into-holes 2 | T366W | T366S-L368A-Y407V | MAbs. |
| knobs-into-holes 3 | S354C, T366W | Y349C, T366S, L368A, | 2017 February-March; 9(2): 182-212; |
| | | Y407V | Atwell et al, J Mol |
| knobs-into-holes 4 | Y349C, T366W | S354C, T366S, L368A, | Biol 1997; 270: 26-35; |
| | | Y407V | Merchant et |
| | | | al, Nat Biotechnol 1998; |
| | | | 16: 677-681 |
| HA-TF | S364H, F405A | Y349T, T394F | Moore et al, MAbs 2011; |
| | | | 3: 546-557 |
| ZW1 | T350V, L351Y, | T350V, T366L, K392L, | Von Kreudenstein et al, MAbs |
| | F405A, Y407V | T394W | 2013; 5: 646-54 |
| CH3 charge pairs (DD- | K392D, K409D | E356K, D399K | Gunasekaran et al, J Biol |
| KK) | | | Chem 2010; 285: 19637-46 |
| IgG1 hinge/CH3 | IgG1: D221E, | IgG1: D221R, P228R, | Strop P et al, J Mol |
| charge pairs (EEE-RRR) | P228E, L368E | K409R | Biol 2012; 420: 204-19 |
| IgG2 hinge/CH3 | IgG2: C223E, | IgG2: C223R, E225R, | |
| charge pairs (EEE- | P228E, L368E | P228R, K409R | |
| RRRR) | | | |
| EW-RVT | K360E, K409W, | Q347R, D399V, F405T | Choi et al, Mol Cancer |
| | | | Ther 2013; 12: 2748-59 |
| EW-RVY_{S-S} | K360E, K409W, | Q347R, D399V, F405T, | Choi et al, Mol |
| | Y349C | S354C | Immunol 2015; 65: 377-83 |
| Biclonic | 366K (+351K) | 351D or E or D at 349, | Geuijen et al, ournal of Clinical |
| | | 368, 349, or 349 + 355 | Oncology 2014; 32: suppl: 560 |
| DuoBody (L-R) 1 | F405L | K409R | Labrijn et al, Nat |
| DuoBody (L-R) 2 | F405L-R409K | WT (R409) | Protoc 2014; 9: 2450-63; |
| | | | Labrijn et al, PNAS 2013; |
| | | | 110(13): 5145-50 |
| SEEDbody | IgG/A chimera | IgG/A chimera | Davis et al, Protein Eng Des |
| | | | Sel 2010; 23: 195-202 |
| BEAT | residues from | residues from TCRβ | Moretti et al, BMC |
| | TCRα interface | interface | Proceedings 2013; 7(Suppl |
| | | | 6): O9 |
| Mixed interface (MI) | IgG-CH3 variants | igA/D/M CH3 variants | Skegro et al, J Biol Chem. 2017 |
| heterodimers | | or IgM CH4 variants | 292(23): 9745-9759. |
| XmAb | E357Q-S364K | L368D-K370S | Moore et al, Methods. 2019 |
| | | | 154: 38-50 |
| DEKK Fc | L351D-L368E | L351K-T366K | De Nardis et al., J Biol Chem. |
| | | | 2017; 292(35): 14706-14717. |
| Charge pair | E356K or E357K | K370E, K409D, K439E | Igawa T, Tsunoda H. |
| | or D399K | | WO2006106905. 2006. |
| KKA-DDW | D356K-D399K- | K392D-K409D- | Zhou et al, WO2014079000A1 |
| | Y407A | T366W | |
| Charge pair | L368E-Y407E | E357K-D399K | Labrijn et al, Nat |
| | | | Review Drug Discovery 2019; |
| | | | 18, 585-608 |
| Knob-hole- | S354C-T366W- | Y349C-T366S-L368A- | Wei et al., Oncotarget. 2017; |
| electrostatic | K409A | Y407-F405K | 8(31): 51037-51049 |
| KA | F405K | K409A | Wei et al., Oncotarget. 2017; |
| | | | 8(31): 51037-51049 |
| PPV-TPP | P395K-P396K- | T394D-P395D-P396D, | Wenjun Zhang US10538595B2 |
| | V397K, P395K- | T394C-P395D-P396D | |
| | P396K-V397C | or T394E-P395E-P396E | |
| | or P395R- | | |
| | P396R-V397R | | |

In some embodiments, bispecific antibody can be generated by post-production assembly from half-antibodies, thereby solving the issues of heavy and light chain mispairing. These antibodies often contain modification to favor heterodimerization of half-antibodies. Exemplary systems include but not limited to the knob-into-hole, IgG1 (EEE-RRR), IgG2 (EEE-RRRR) (Strop et al. J Mol Biol (2012)) and DuoBody (F405L-K409R), listed in Table A. In such case, half-antibody is individually produced in separate cell line and purified. The purified antibodies were then subjected to mild reduction to obtain half-antibodies, which were then assembled into bispecific antibodies. Heterodimeric bispecific antibody was then purified from the mixture using conventional purifications methods.

In some embodiments, strategies on bispecific antibody generation that do not rely on the preferential chain pairing can also be employed. These strategies typically involve introducing genetic modification on the antibody in such a manner that the heterodimer will have distinct biochemical or biophysical properties from the homodimers; thus the post-assembled or expressed heterodimer can be selectively purified from the homodimers. One example was to introduce H435R/Y436F in IgG1 CH3 domain to abolish the Fc binding to protein A resin and then co-express the H435R/Y436F variant with a wildtype Fc. The resulting homodimeric antibodies containing two copies of H435R/Y436F cannot bind to the Protein A column, while heterodimeric antibody comprising one copy of H435R/Y436F mutation will have a decreased affinity for protein A as compared to the strong interaction from homodimeric wildtype antibody (Tustian et al Mabs 2016). Other examples include kappa/lambda antibody (Fischer et al., Nature Communication 2015) and introduction of differential charges (E357Q, S267K or N208D/Q295E/N384D/Q418E/N421D) on the respective chains (US 2018/0142040 A1; (Strop et al. J Mol Biol (2012)).

In some embodiments, bispecific antibody can be generated via fusion of an additional binding site to either the heavy or light chain of an immunoglobulin. Examples of the additional binding site include but not limited to variable regions, scFv, Fab, VHH, and peptide.

In some embodiments, the heterodimeric mutations and/or mutations to modify Fc gamma receptor binding resulted in reduction of Fc stability. Therefore, additional mutation(s) was added to the Fc region to increase its stability. For example, one or more pairs of disulfide bonds such as A287C and L306C, V259C and L306C, R292C and V302C, and V323C and I332C are introduced into the Fc region. Another example is to introduce S228P to IgG4 based bispecific antibodies to stabilize the hinge disulfide. Additional example includes introducing K338I, A339K, and K340S mutations to enhance Fc stability and aggregation resistance (Gao et al, 2019 Mol Pharm. 2019; 16:3647).

### Antigen Binding Molecules

In some embodiments, the fusion protein binds human CD8, and the binding of the fusion protein to CD8 does not block the interaction of CD8 with MEC class I. In some embodiments, the antigen binding molecule of the present disclosure binds to an epitope on CD8α wherein the binding of the antigen binding molecule to CD8α does not block the interaction of CD8αα or CD8αβ with MHC class I molecules on target cells or antigen presenting cells. In some embodiments, the antigen binding molecule of the present disclosure binds to an epitope on CD8β wherein the binding of the antigen binding molecule to CD8β does not block the interaction of CD8αβ with MEC class I molecules on target cells or antigen presenting cells. In some embodiments, whether an anti-CD8 antibody or fusion protein of the present disclosure blocks the interaction of CD8 with MEC class I can be assayed, e.g., by assaying activation status of CD8+ T cells (e.g., upon antigen stimulation) in the presence or absence of the anti-CD8 antibody or fusion protein. For an exemplary assay and conditions, see, e.g., Example 3.

In some embodiments, an anti-CD8 antibody or fusion protein of the present disclosure comprises a VH domain comprising the sequence of EVQLVESGGGLVQPGRSLKLSCAASGFTFSNYYMAWVRQAPTKGLEWVAYINTGGGTT YYRDSVKGRFTISRDDAKSTLYLQMDSLRSEDTATYYCTTAIGYYFDYWGQGVMVTVS S (SEQ ID NO:10) and a VL domain comprising the sequence of DIQLTQSPASLSASLGETVSIECLASEDIYSYLAWYQQKPGKSPQVLIYAANRLQDGVPS RFSGSGSGTQYSLKISGMQPEDEGDYFCLQGSKFPYTFGAGTKLELK (SEQ ID NO:11).

In some embodiments, an anti-CD8 antibody or fusion protein of the present disclosure comprises a VH domain comprising the sequence of EVKLQESGPSLVQPSQTLSLTCSVSGFSLISDSVHWVRQPPGKGLEWMGGIWADGSTDY NSALKSRLSISRDTSKSQGFLKMNSLQTDDTAIYFCTSNRESYYFDYWGQGTMVTVSS (SEQ ID NO:12) and a VL domain comprising the sequence of DIQMTQSPASLSASLGDKVTITCQASQNIDKYIAWYQQKPGKAPRQLIHYTSTLVSGTPS RFSGSGSGRDYSFSISSVESEDIASYYCLQYDTLYTFGAGTKLELK (SEQ ID NO:13). In some embodiments, an anti-CD8 antibody or fusion protein of the present disclosure comprises a VH domain comprising the sequence of EVKLQESGPSLVQPSQTLSLTCSVSGFSLISDSVHWVRQPPGKGLEWMGGIWADGSTDY NSALKSRLSISRDTSKSQGFLKMNSLQTDDTAIYFCTSARESYYFDYWGQGTMVTVSS (SEQ ID NO:14) and a VL domain comprising the sequence of DIQMTQSPASLSASLGDKVTITCQASQNIDKYIAWYQQKPGKAPRQLIHYTSTLVSGTPS RFSGSGSGRDYSFSISSVESEDIASYYCLQYATLYTFGAGTKLELK (SEQ ID NO:15). In some embodiments, an anti-CD8 antibody or fusion protein of the present disclosure comprises a VH domain comprising the sequence of EVQLVESGGALVQPGRSLKLSCAASGLTFSDCYMAWVRQTPTKGLEWVSYISSDGGST YYGDSVKGRFTISRDNAKSTLYLQMNSLRSEDMATYYCACATDLSSYWSFDFWGPGT MVTVSS (SEQ ID NO:16) and a VL domain comprising the sequence of DIQMTQSPSSLPVSLGERVTISCRASQGISNNLNWYQQKPDGTIKPLIYHTSNLQSGVPSR FSGSGSGTDYSLTISSLEPEDFAMYYCQQDATFPLTFGSGTKLEIK (SEQ ID NO:17).

In some embodiments, the antigen binding molecule of the present disclosure binds to an epitope on PD1 wherein the binding of the antigen binding molecule to PD1 does not block the interaction of PD1 with PD-L1 expressed on target cells or other immune cells. Such fusion proteins are particularly useful as they can be administered as therapeutics in combination with anti-PD1 therapeutic antibodies, including but not limited to nivolumab, pembrolizumab, and cemiplimab.

In some embodiments, the antigen binding molecule of the present disclosure binds to an epitope on PD1 wherein the binding of the antigen binding molecule to PD1 blocks the interaction of PD1 with PD-L1 on target cells or other immune cells. Such fusion proteins are particularly useful as they can be administered as therapeutics in combination with anti-PDL1 therapeutic antibodies, including but not limited to atezolizumab, avelumab, and durvalumab.

Certain aspects of the present disclosure relate to methods of treating cancer or chronic infection. In some embodiments, the methods comprise administering an effective amount of a fusion protein, or a pharmaceutical composition comprising the fusion protein and a pharmaceutically acceptable carrier, to a patient. In some embodiments, the patient in need of said treatment has been diagnosed with cancer.

In some embodiments, the fusion protein or composition is administered in combination with a T cell therapy, cancer vaccine, chemotherapeutic agent, or immune checkpoint inhibitor (ICI). In some embodiments, the chemotherapeutic agent is a kinase inhibitor, antimetabolite, cytotoxin or cytostatic agent, anti-hormonal agent, platinum-based chemotherapeutic agent, methyltransferase inhibitor, antibody, or anti-cancer peptide. In some embodiments, the immune checkpoint inhibitor targets PD-L1, PD-1, CTLA-4, CEACAM, LAIR1, CD160, 2B4, CD8β, CD86, CD276, VTCN1, HVEM, KIR, A2AR, MEW class I, MHC class II, GALS, adenosine, TGFR, OX40, CD137, CD40, IDO, CSF1R, TIM-3, BTLA, VISTA, LAG-3, TIGIT, IDO, MICA/B, LILRB4, SIGLEC-15, or arginase, including without limitation an inhibitor of PD-1 (e.g., an anti-PD-1 antibody), PD-L1 (e.g., an anti-PD-L1 antibody), or CTLA-4 (e.g., an anti-CTLA-4 antibody). Examples of T cell therapies include, without limitation, CD4+ or CD8+ T cell-based therapies, adoptive T cell therapies, chimeric antigen receptor (CAR)-based T cell therapies, tumor-infiltrating lymphocyte (TIL)-based therapies, autologous T cell therapies, and allogeneic T cell therapies. Exemplary cancer vaccines include, without limitation, dendritic cell vaccines, vaccines comprising one or more polynucleotides encoding one or more cancer antigens, and vaccines comprising one or more cancer antigenic peptides.

In some embodiments, a fusion protein of the present disclosure is part of a pharmaceutical composition, e.g., including the fusion protein and one or more pharmaceutically acceptable carriers. Pharmaceutical compositions and formulations as described herein can be prepared by mixing the active ingredients (such as a fusion protein) having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives; low molecular weight (less than about 10 residues) polypeptides; proteins, such as serum albumin, gelatin, or immunoglobulins; hydrophilic polymers such as polyvinylpyrrolidone; amino acids; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). In some embodiments, a fusion protein of the present disclosure is lyophilized.

### Enumerated Embodiments

The following enumerated embodiments are representative of some aspects of the invention.
1. A fusion protein comprising two moieties, wherein:
i) The first moiety is a polypeptide comprising an antibody heavy chain VH—CH1-hinge-CH2-CH3 monomer wherein VH is a variable heavy chain and CH2-CH3 is a Fc domain, an antibody light chain VL-CL wherein VL is a variable light chain and CL is a constant light chain, and a mutant IL-2 polypeptide, wherein the N-terminus of the mutant IL-2 polypeptide is fused to the C-terminus of the Fc domain via a linker;
ii) The second moiety is a polypeptide comprising an antibody heavy chain VH—CH1-hinge-CH2-CH3 monomer and an antibody light chain VL-CL;
and wherein, both the first and second moiety bind to an epitope on one antigen selected from the following group: human CD8α, human CD8β, and human PD1.
2. A fusion protein comprising two moieties, wherein:
i) The first moiety is a polypeptide comprising an antibody hinge-CH2-CH3 monomer wherein CH2-CH3 is a Fc domain, and a mutant IL-2 polypeptide, wherein the N-terminus of the mutant IL-2 polypeptide is fused to the C-terminus end of the Fc domain via a linker;
ii) The second moiety is a polypeptide comprising an antibody heavy chain VH—CH1-hinge-CH2-CH3 monomer and an antibody light chain VL-CL;
and wherein the second moiety binds to an epitope on one antigen selected from the following group: human CD8α, human CD8β, and human PD1.
3. A fusion protein comprising two moieties, wherein:
i) The first moiety is a polypeptide comprising an antibody hinge-CH2-CH3 monomer wherein CH2-CH3 is a Fc domain, and a mutant IL-2 polypeptide, wherein the C-terminus of the mutant IL-2 polypeptide is fused to the N-terminus end of the Fc domain via a linker;
ii) The second moiety is a polypeptide comprising an antibody heavy chain VH—CH1-hinge-CH2-CH3 monomer and an antibody light chain VL-CL;
and wherein the second moiety binds to an epitope on one antigen selected from the following group: human CD8α, human CD8β, and human PD1.
4. The fusion protein of any one of embodiments 1 to 3 wherein said mutant IL-2 polypeptide exhibits reduced binding affinity by 50% or more to IL-2Rα polypeptide having an amino acid sequence depicted in FIG. 1B, compared to the binding affinity of the wild-type IL-2 polypeptide with an amino acid sequence depicted in FIG. 1A, and reduced binding affinity by 50% or more to IL-2Rα polypeptide having an amino acid sequence depicted in FIG. 1C, compared to the binding affinity of the wild-type IL-2 polypeptide with an amino acid sequence depicted in FIG. 1A.
5. The fusion protein of embodiment 4 wherein said mutant IL-2 polypeptide further exhibits reduced binding affinity by 50% or more to IL-2Rγ polypeptide having an amino acid sequence depicted in FIG. 1D, compared to the binding affinity of the wild-type IL-2 polypeptide with an amino acid sequence depicted in FIG. 1A.
6. The fusion protein of embodiment 4 or 5 wherein said mutant IL-2 polypeptide has two or more amino acid substitutions relative to the wild-type IL-2 amino acid sequence as depicted in FIG. 2 and selected from a group of: Q11, E15, H16, L18, L19, D20, Q22, R38, F42, K43, Y45, E62, P65, E68, V69, L72, N88, V91, 192, T123, Q126, S127, I129, S130.
7. The fusion protein of embodiment 6 wherein said mutant IL-2 polypeptide further comprises the amino acid mutation C125A to improve its biophysical properties compared to wild-type IL-2.
8. The fusion protein of embodiment 6 or 7 wherein said first and second Fc domains contain the following Fc mutations to decrease effector function according to EU numbering: L234A, L235A, G237A, and K322A.
9. The fusion protein of embodiment 6 or 7 wherein said first and second Fc domains contain the following amino acid substitutions to facilitate heterodimeric formation: Y349C/T366W (knob) and S354C, T366S, L368A and Y407V (hole).
10. One or more isolated polynucleotides encoding the mutant IL-2 polypeptides or fusion protein of any one of embodiments 1-9.
11. One or more vectors, particularly expression vectors, comprising the polynucleotides of embodiment 10.
12. A host cell comprising the polynucleotides of embodiment 10.
13. A pharmaceutical composition comprising the fusion proteins according to any one of embodiments 1-9 and a pharmaceutically acceptable carrier.
14. The fusion proteins of any one of embodiments 1-9 for use as a medicament.
15. A method of treating cancer or chronic infection comprising administering a composition according to any of embodiments 1-9 and 13 to a patient.
16. A method of treating cancer comprising administering a composition according to any of embodiments 1-9 and 13 to a patient in combination with a T cell therapy or cancer vaccine.

### EXAMPLES

### Example 1

### Recombinant DNA Techniques

Techniques involving recombinant DNA manipulation were previously described in Sambrook et al., Molecular cloning: A laboratory manual; Cold Spring Harbor Laboratory Press, Cold Spring Harbor, N.Y., 1989. All reagents were used according to the manufacturer's instructions. DNA sequences were determined by double strand sequencing.

### Gene Synthesis

Desired gene segments were either generated by PCR using appropriate templates or synthesized at Thermo Scientific (Pleasanton, Calif.), ATUM (Newark, Calif.), Genewiz (South Plainfield, N.J.), or GeneScript (Piscataway, N.J.) from synthetic oligonucleotides. The gene segments flanked by designed restriction endonuclease cleavage sites were digested out and later cloned into their respective expression vectors. DNA was purified from transformed bacteria and concentration determined by UV visible spectroscopy. DNA sequencing was used to confirm the DNA sequences of the subcloned gene fragments.

### Isolation of Antibody Genes

Antibodies binding to CD8 or PD1 antigens were generated using either in vitro display system or in vivo immunizations. For in vitro display method, a non-immune human antibody phage library was panned for 5 to 6 rounds to isolate antibodies against the target antigen. After the panning, individual phage clones that exhibited specific binding to target antigen over non-specific antigens in ELISA were identified. DNA fragments of heavy and light chain V-domain of the specific binders were subsequently cloned and sequenced. Meanwhile, antibodies were also generated from immunizing mice and llamas with the recombinant form of the antigens. From the mouse immunization, hybridoma method was used to isolate the antibody. Briefly, after immunization, B cells from spleen and/or lymph nodes were fused with a myeloma cell line to generate the hybridoma cells. Hybridoma clones were then individually screened using ELISA to identify the clones expressing antibodies specific for the antigen. Finally, DNA fragments of heavy and light chain V-domain of the antibody were cloned from the specific hybridoma and later sequenced. For the llama immunization, antibody genes were cloned from peripheral B cells and ligated into the phagemid vector to generate a phage display antibody library. Antibodies were then isolated through panning the phage library against the antigens of interest. After the panning, individual phage clones that exhibited specific binding to target antigen over non-specific antigens were identified using ELISA. DNA fragments of heavy and light chain V-domain of the specific binders were then subsequently cloned and sequenced. Antibodies from non-human origins (mouse and llama) were then humanized to remove non-human framework and complementarity-determining region mutations.

### Cloning of Fusion Constructs

General information regarding the nucleotide sequences of human immunoglobulins light and heavy chains is given in: IMGT® (the international ImMunoGeneTics information System®) from Lefranc et al. IMGT®, the international ImMunoGeneTics information System® 25 years on. Nucleic Acids Res. 2015 January; 43. The amplified DNA fragments of heavy and light chain V-domains were inserted in frame into the human IgG1 containing mammalian expression vector. The IL-2 portions of the constructs were cloned in frame with the heavy chain using a (G4S)3 15-mer linker between the C-terminus of the IgG heavy chain and the N-terminus of IL-2. The C-terminal lysine residue of the IgG heavy chain was eliminated after fusing the IL-2 portion. To generate the construct in which a single IL-2 gene was fused to a full IgG, two heavy chain plasmids needed to be constructed and transfected for heterodimerization facilitated by a knob-into-hole modification in the IgG CH3 domains. The “hole” heavy chain connected to the IL-2 portion carried the Y349C, T366S, L368A and Y407V mutations in the CH3 domain, whereas the unfused “knob” heavy chain carried the S354C and T366W mutations in the CH3 domain (EU numbering). To abolish FcγR binding/effector function and prevent FcR co-activation, the following mutations were introduced into the CH2 domain of each of the IgG heavy chains: L234A/L235A/G237A (EU numbering). The expression of the antibody-IL-2 fusion constructs was driven by an CMV promoter and transcription terminated by a synthetic polyA signal sequence located downstream of the coding sequence.

Preparation of Fusion Proteins with IL-2 Polypeptides

Constructs encoding fusion proteins with IL-2 polypeptides as used in the examples were produced by co-transfecting exponentially growing Expi293 cells with the mammalian expression vectors using polyethylenimine (PEI). Briefly, IL-2 fusion constructs were first purified by affinity chromatography using a protein A matrix. The protein A column was equilibrated and washed in phosphate-buffered saline (PBS). The fusion constructs were eluted with 20 mM sodium citrate, 50 mM sodium chloride, pH 3.6. The eluted fractions were pooled and dialyzed into 10 mM IVIES, 25 mM sodium chloride pH 6. The proteins were further purified using ion-exchange chromatograph (Mono-S, GE Healthcare) to purify the heterodimers over the homodimers. After loading the protein, the column is washed with 10 mM MES 25 mM sodium chloride pH 6. The protein was then eluted with increasing gradient of sodium chloride from 25 mM up to 500 mM MES in 10 mM MES pH 6 buffer. The major eluent peak corresponding to the heterodimer was collected and concentrated. The purified protein was then polished by size exclusion chromatography (Superdex 200, GE Healthcare) in PBS.

The protein concentration of purified IL-2 fusion constructs was determined by measuring the optical density (OD) at 280 nm, using the molar extinction coefficient calculated on the basis of the amino acid sequence. Purity, integrity and monomeric state of the fusion constructs were analyzed by SDS-PAGE in the presence and absence of a reducing agent (5 mM 1,4-dithiothreitol) and stained with Coomassie blue (SimpleBlue™ SafeStain, Invitrogen). The NuPAGE® Pre-Cast gel system (Invitrogen) was used according to the manufacturer's instructions (4-20% Tris-glycine gels or 3-12% Bis-Tris). The aggregate content of immunoconjugate samples was analyzed using a Superdex 200 10/300 GL analytical size-exclusion column (GE Healthcare).

pSTAT5 and Ki-67 Assays to Measure Selective Activation of Antigen-Expressing Blood Immune Cells

Activity of IL-2 fusion proteins was determined in an assay with human peripheral blood mononuclear cells (PBMCs) measuring the phosphorylation of STAT5. PBMCs were isolated from blood of healthy donors using Ficoll-Paque Plus (GE Healthcare) and red blood cells were lysed using ACK lysis buffer (Gibco) according to manufacturer's instructions. Typically, PBMCs were resuspended in serum-free RPMI1640 media at 2×10⁶ cells/ml and aliquoted into 96-well U-bottom plates (50 μl per well). IL-2 fusion proteins and control proteins, such as recombinant human IL-2 and control (HA-targeted) fusion proteins, were diluted to desired concentrations and added to wells (50 μl added as 2× stimulus). Incubation was typically performed for 30 min at 37° C., after which it was stopped with 100 μl pre-warmed 4% PFA (2% final) for 10 min at 37° C. Cells were then stained with antibodies against surface markers: CD45 (clone HI30), CD3 (UCHT1, BD Biosciences), CD8α (SK1, Biolegend; RPA-T8, Biolegend), CD4 (RPA-T4, Biolegend), and CD25 (M-A251, Biolegend). Cells were washed 2× with wash buffer (2% FBS in PBS) and fixed with 4% PFA at room temperature for 10 min. After fixation, cells were permeabilized in pre-chilled Phosflow Perm buffer III (BD Biosciences) according to manufacturer's protocol. After permeabilization, cells were stained with antibodies against intracellular markers (pSTAT5 [pY694], clone 47, BD Biosciences, and/or perforin, clone δG9, BD Biosciences) and analyzed on a flow cytometer. Data were expressed as percent pSTAT5 positive, and in some cases as pSTAT5 mean fluorescence intensity (MFI), and imported into GraphPad Prism to determine EC₅₀ values for each construct.

To measure cellular changes induced by IL-2 fusion proteins further downstream from pSTAT5 such as proliferation, a flow cytometry assay was used to detect the expression of the intracellular proliferation marker Ki-67. Briefly, PBMCs were isolated as described above and incubated in the presence of IL-2 fusion proteins and controls in serum-supplemented RPMI1640 (10% FBS) for 4 to 6 days or serum free AIM V media (Gibco). Staining for Ki-67 (clone Ki-67, Biolegend) was performed with Foxp3/Transcription Factor Staining Buffer Set (Thermo Fisher Scientific) according to manufacturer's protocol. Data were expressed as percent Ki-67 positive and imported into GraphPad Prism to determine EC₅₀ values for each construct when possible.

pSTAT5 and Ki67 Assays to Measure Activation of Mouse Immune Cells

Splenocytes were isolated from spleens of B6 mice by placing a spleen onto a 70 mM strainer and using a plunger to wash the cells with PBS through the strainer. Red blood cells were lysed with ACK lysis buffer and cells resuspended at 20×106/m1 of RPMI media. Cells were plated in U-bottom plates at 50 ml per well. IL-2 fusion proteins and control proteins were added to cells (50 μl as 2× stimulus). CD49b antibody (5 ml, DX5 clone) was added to each well prior to incubating the cells at 37° C. for 30 min. Cells were fixed with 8% PFA (4% final). Cells were washed 2× with PBS-2% FBS and resuspended in 75 ml Phosflow Perm buffer III buffer and incubated for 1 hr at 4° C. Cells were washed 3× with PBS-2% FBS and stained in 50 μl of FACS buffer containing antibodies against CD3 (17A2), CD4 (GK1.5), CD8α (53-6.7), CD8b (YTS156.7.7), CD25 (7D4), and pSTAT5 (clone 47). Samples were washed 2× and analyzed on a flow cytometer. For Ki67 assay, 1×105 splenocytes were plated in 96 well U-bottom plates in RPMI media supplemented with 10% FBS and cultured at 37° C. for 5 days prior to staining for Ki-67. Briefly, cells were surface stained with antibodies against CD3 (145-2C11), CD4 (GK1.4), CD8 (53-6.7), CD25 (PC61), and NK1.1 (PK136), then fixed and permeabilized using Foxp3/Transcription Factor Staining Buffer Set (Thermo Fisher Scientific) according to manufacturer's protocol. Ki67 antibody (clone 16A8) was added for 45 min at 4° C., after which the cells were washed and analyzed on a flow cytometer.

### Binding Affinity Determination by Surface Plasmon Resonance (SPR) for IL-2Rα and IL-2Rβ

Kinetic rate constants (kon and koff) as well as affinity (Ku) of IL-2 fusion proteins for human and cynomolgus CD8α, CD8β and PD1 antigens, and for human IL-2Rα and IL-2Rβ were measured by surface plasmon resonance (SPR) using a BIAcore (GE Healthcare) (10 mM phosphate, 150 mM sodium chloride pH 7.4, 0.005% Tween 20) at 37° C. Briefly, to determine the affinities, IL-2 fusion proteins were captured onto the CM4 sensor chip via their Fc by a covalently immobilized anti-human Fc capture antibody at 0.75 μg/mL and a flow rate of 10 μL/min for 30s. Antibody was not captured on flow cell 1 to serve as a reference surface. Various concentrations of antigens (3-fold dilution from 1m1V1 to 1.4 nM to) or IL-2 receptors (3-fold dilution from 3 mM to 12 nM) were injected as analytes over the captured IL-2 fusion proteins. Association phase was recorded for 120s, dissociation phase for 600s. Sensorgrams were double-referenced and fit globally to the 1:1 Langmuir with mass transport model to determine the association rates (kon), dissociation rates (koff) and equilibrium dissociation constant (KD=koff/kon) using Biacore Evaluation Software.

In an alternative format of the assay antigens were first captured on the chip and IL-2 fusion proteins then injected as analytes. In this case, in-house generated or commercially purchased histidine-tagged IL-2 receptors were diluted in running buffer to 0.125 μg/mL and captured on the CM4 sensor chip amine-coupled with anti-HIS antibody for 1 minute at 10 μL/min. IL-2 receptors were not captured on flow cell 1 to serve as a reference surface. IL-2 fusion proteins from 1000 nM to 4.1 nM (3-fold dilution series) were injected over flowcell 1 and 2 for 2 min and allowed to dissociate for 1 min. Surfaces were regenerated with two 30s injections of 10 mM Glycine pH 1.7 between analysis cycles. Sensorgrams were double-referenced and fit globally to the 1:1 Langmuir to determine kon, koff, and KD using Biacore Evaluation Software.

### Example 2: Ability of IL-2 and IL-2 Variants to Activate Splenic Cell Subsets

This example describes experiments to test the capacity of IL-2 fusion compounds to activate STAT5 in mouse NK cells and to induce NK-mediated toxicity in mice.

The ability of IL-2 and IL-2 variants with reduced binding to CD25/IL2Rα to activate splenic cell subsets was tested with a STAT5 assay. IL-2 and a previously published IL-2 variant (IL-2v) fused to control antibody (xHA), xHA-IL-2v, were used to stimulate mouse splenocytes containing CD8 T cells, CD4 T cells and NK cells. STAT5 activation in different splenic subsets was measured by flow cytometry, as described in Example 1. CD8 T cells were identified in the CD3+CD4− gate/subset, Treg cells were identified as CD3+CD4+CD25+, and NK cells were identified as CD3-CD49b+.

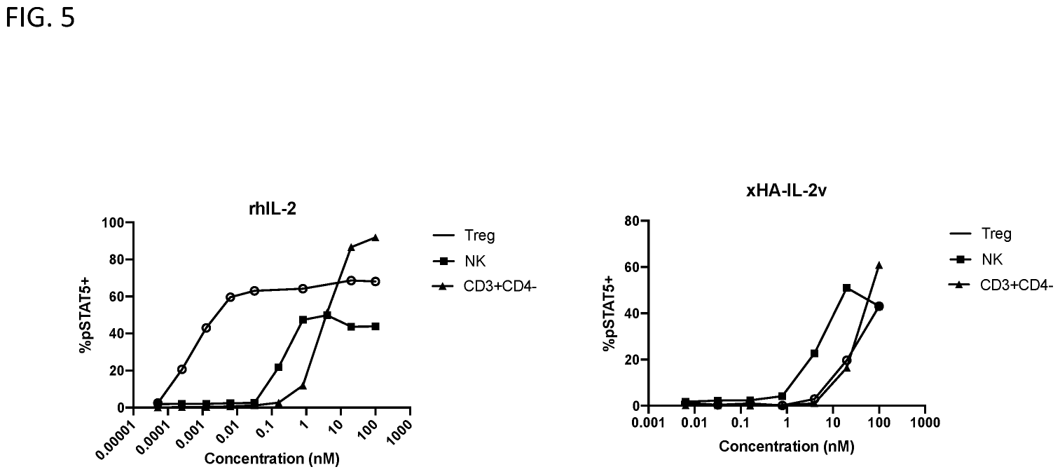

FIG. 5 shows the results of this experiment. NK cells were ˜10× more sensitive to IL-2 stimulation than CD8+ T cells, while Treg cells were the most sensitive (FIG. 5 at left). In the case of IL-2v, due to its lowered binding to CD25, NK cells were ˜10× more sensitive to IL-2v stimulation than both CD8 T cells and Treg cells (FIG. 5 at right).

To test for NK-cell induced toxicity, body weight loss was measured in mice treated with an IL-2 variant with reduced binding CD25/IL2Ra. NK-cell induced toxicity upon treatment with the IL-2 variants can manifest as body weight loss. For this experiment, B6 mice of 8-10 weeks of age were injected subcutaneously with a single dose of indicated compounds and their body weights recorded daily. xHA-IL-2v was dosed at 1 mg/kg or 5 mg/kg together with anti-PD1 (xPD1) at 2.5 mg/kg, while TAg-IL-2v was dosed alone at 5 mg/kg. NK cells were depleted with anti-NK1.1 antibody (PK136 clone) at 200 mg/mouse i.p. The depleting antibody was injected two days prior to TAg-IL-2v dosing and one day after dosing to maintain depletion.

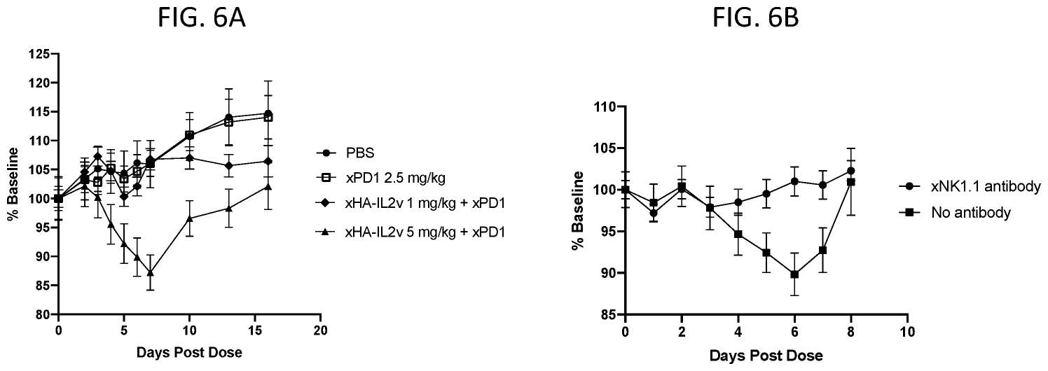

FIGS. 6A & 6B show the results of these experiments. IL-2 variant (IL-2v) fused to either control xHA antibody (xHA-IL-2v) or FAP antibody (TAg-IL-2v) induced body weight loss in mice (FIG. 6A). This body weight loss was mediated by NK cells as evident in mice where NK cells were depleted with NK1.1 antibody (FIG. 6B). Such toxicity mediated by NK cells could limit maximum tolerated dose of IL-2-based therapeutics in humans. Maximum tolerated dose for control antibody targeted-IL2v or tumor antigen targeted-IL2v in mice was well below 5 mg/kg.

### Example 3: Characterization of Anti-Mouse CD8 Antibodies

This example describes the characterization of anti-mouse CD8 antibodies.

### Results

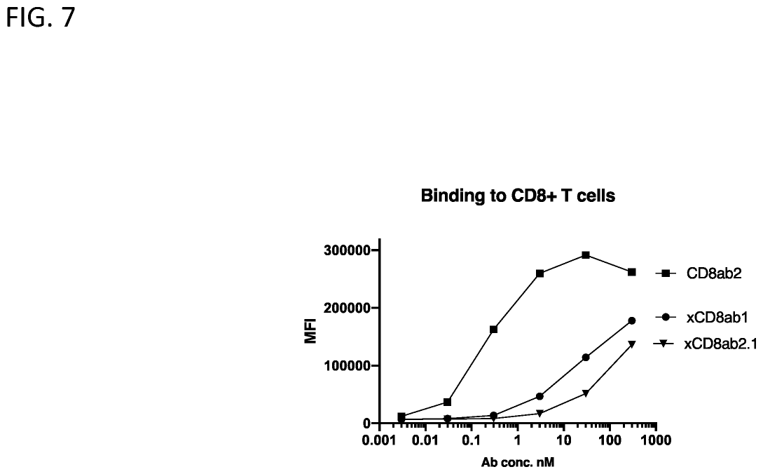

The binding affinity of anti-mouse CD8 antibodies was determined through flow cytometry analysis. Fresh splenocytes were incubated with the either xCD8ab1 (clone 2.43), xCD8ab2 (clone YTS156.7.7) or xCD8ab2.1 for 2 hours at 4° C. xCD8ab1 (clone 2.43) and xCD8ab2 (clone YTS156.7.7) sequences have been previously published. The xCD8ab2.1 clone is derived from xCD8ab2 by introduction of mutations N95A (VH) and D92A (VK). After incubation with the anti-mouse CD8 antibody, the cells were then stained with antibodies against CD3, CD4, CD8 and anti-hFc (HP6017, Biolegend), the latter of which was used to measure the binding of CD8-IL2 fusion containing hFc. Stained cells were washed and analyzed by flow cytometry and mean fluorescence intensity (MFI) of staining with anti-hFc was used to denote binding. As shown in FIG. 7, xCD8ab2 had a higher affinity for CD8+ T cells than xCD8ab1. xCD8ab2.1 is a lower affinity variant of xCD8ab2 that was generated by introducing two mutations (N95A (VH) and D92A (VK)) in xCD8ab2.

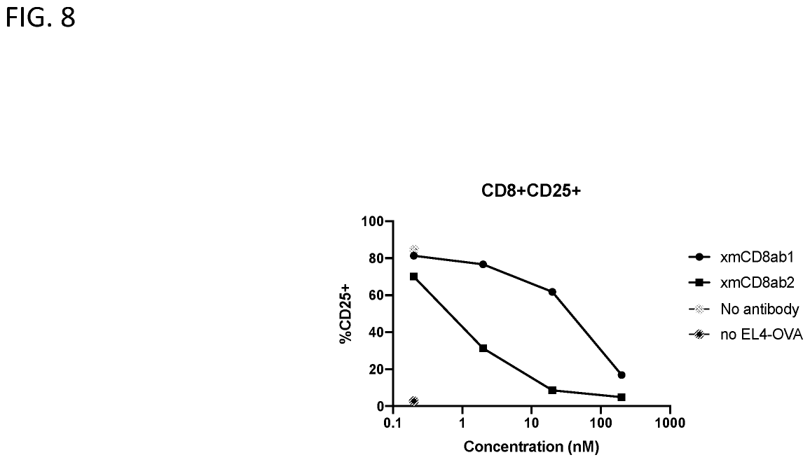

The MHC blocking status for anti-mouse CD8 antibodies was also tested. Binding of certain CD8 antibodies can interfere with interaction of CD8 molecules on T cells and MHC molecules on antigen presenting cells or tumor cells, thereby inhibiting T cell activation. For this experiment, CD8+ T cells were purified from splenocytes from OT-I transgenic mice and co-cultured with EL-4-OVA cancer line (ATCC), at 100,000 cells each for 24 hr. The cells were analyzed for upregulation of activation markers such as CD25 and CD69 by cell surface staining and flow cytometry. As shown in FIG. 8, both xCD8ab1 and xCD8ab2 blocked T cell activation, suggesting that these antibodies interfered with and blocked the interaction of CD8 with MHC. xCD8ab2 blocked T cell activation more potently, correlating with its higher binding affinity to CD8.

### Example 4: Characterization of IL-2 Muteins Fused to Anti-CD8 Antibodies

This example describes the characterization of IL-2 muteins fused to CD8 antibodies.

### Methods

### B16 Mouse Tumor Model

C57BL6 female mice (Jackson Labs) at 8-10 weeks of age were housed and acclimated at the vivarium facility. Cultured B16.F10 cells (ATCC, CRL-6475) were harvested and resuspended in serum free media (1×DMEM, Sigma D6429) at 5×106 cells/mL for implantation. Mice were shaved and 5×10\hat{ }5 cells (100 μL) are implanted subcutaneously above rear hind leg. Tumors were measured until tumor volume of 60-120 mm3 was reached at approximately 8 days post implantation (TV=width*width*length*0.5). Mice were then randomized into groups by tumor volume. Each mouse was weighed and dosed subcutaneously under the scruff of the neck. Tumor volumes and body weights were measured every 3-4 days until either end of study (30-40 days post initial dose) or max tumor volume (2000 mm³) was reached. At end of study mice were euthanized by CO2 with appropriate secondary euthanasia.

### CT26 Mouse Tumor Model

BALB/c female mice were shaved, and 2×10⁵ cells (100 μL) of cultured CT26 wt cells (ATCC, CRL-2638) were implanted subcutaneously above the rear hind leg. Tumor volume was measured until it reached 60-120 mm³ was reached at approximately 8 days post implantation (TV=width*width*length*0.5). Each mouse was then weighed and dosed subcutaneously with the indicated compound (9 mice per group).

Analysis of CD8+ T Cells in Blood and Tumor of Mice with B16 Tumor

B6 mice were injected with B16 tumor cells and tumors allowed to grow to 200-250 mm³ before they were dosed with indicated IL-2 fusions at 1 mg/kg. Tumors were removed day 5 post dose, digested to single cells and profiled by flow cytometry to detect CD8+ T cells and NK cells (NK1.1+CD3-). Briefly, tumors were digested using Mouse Tumor Dissociation Kit (Miltenyi Biotec, 130-096-730) in Miltenyi Gentle MACS C tubes according to manufacturer's protocol. Isolated cells were counted and 10×10⁶ cells were stained with antibodies against CD45, CD3, CD4, CD8, CD25, and CD49b. Blood was also collected from mice (50 μl), lysed with ACK lysis buffer, washed with wash buffer (PBS/0.5% BSA/2 mM EDTA) and stained with lineage markers to identify CD8+ T cells and NK cells. Cell were analyzed on a flow cytometer.

### Results

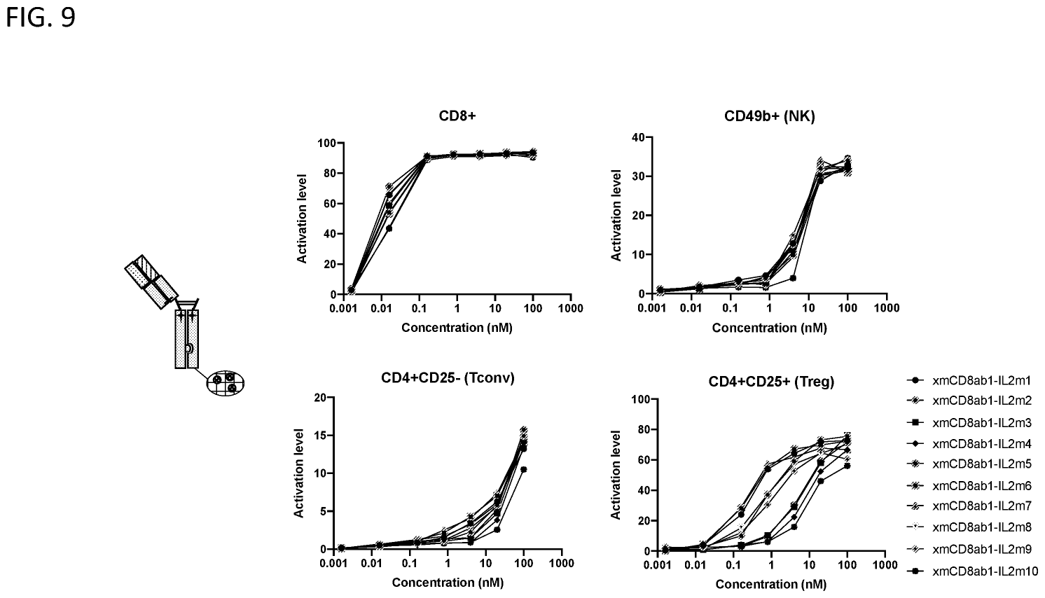

IL-2 mutein variants fused to CD8 antibodies were tested for selective targeting of CD8+ T cells over other immune cells expressing IL-2R. IL-2 muteins were fused to a previously published anti-mouse CD8 antibody, xmCD8ab1 (2.43 clone) in a B format. Mouse splenocytes were treated with the IL-2 mutein fusions and a STAT5 assay was performed as described in Example 1. Table 1 and FIG. 9 summarize the results of this experiment.

IL-2 muteins fused to CD8 antibody in format B selectively targeted CD8+ T cells over other cells that express IL-2R, including NK cells. Activity on Tregs was used as a proxy for IL-2Rα/CD25 binding, since Tregs express CD25 while other cells do not. Other sequences also preferentially targeted CD8 T cells over NK cells but had increased activity on Tregs as a result of higher CD25 binding. Given that activity of IL-2 in this assay is <0.001 nM (panel A of FIG. 5), all of sequences included in Table 1 showed at least 50% reduced binding to IL2Rα/CD25. Furthermore, several sequences were identified with lowest activity on Tregs (m3, m4, m5, m10, as indicated in Table 1).

TABLE 1
Identification of IL-2 muteins fused to CD8 antibodies with lowest
selective activity on Tregs and highest activity on CD8 T cells,
as measured by STAT5 assay (% pSTAT5+).

| IL-2 mutein fused to CD8 | | STAT5 assay (% pSTAT5+) | | | |
|---|---|---|---|---|---|
| antibody xmCD8ab1 (format B) | | EC₅₀ | EC₅₀ | EC₅₀ | EC₅₀ |
| IL-2 mutein | Mutations | (CD8) | (NK) | (Tconv) | (Treg) |
| IL-2m1 | F42A | 0.0047 | 7.08 | 36.7 | 0.308 |
| IL-2m2 | R38A, F42A | 0.0046 | 6.93 | 71.6 | 0.979 |
| IL-2m3 | R38D, F42A | 0.0071 | 6.55 | 133.3 | 7.78 |
| IL-2m4 | R38E, F42A | 0.0142 | 7.69 | 244 | 9.11 |
| IL-2m5 | F42A, E62Q | 0.0030 | 6.42 | 139.3 | 6.41 |
| IL-2m6 | F42A, E68A | 0.0092 | 7.71 | 29.9 | 0.246 |
| IL-2m7 | F42A, E68Q | 0.0093 | 5.81 | 28.7 | 0.195 |
| IL-2m8 | F42A, E68K | 0.0095 | 9.23 | 57.1 | 0.677 |
| IL-2m9 | F42A, E68R | 0.0064 | 5.38 | 72.1 | 0.894 |
| IL-2m10 | R38A, F42K | 0.0146 | 12.8 | 1399 | 9.93 |

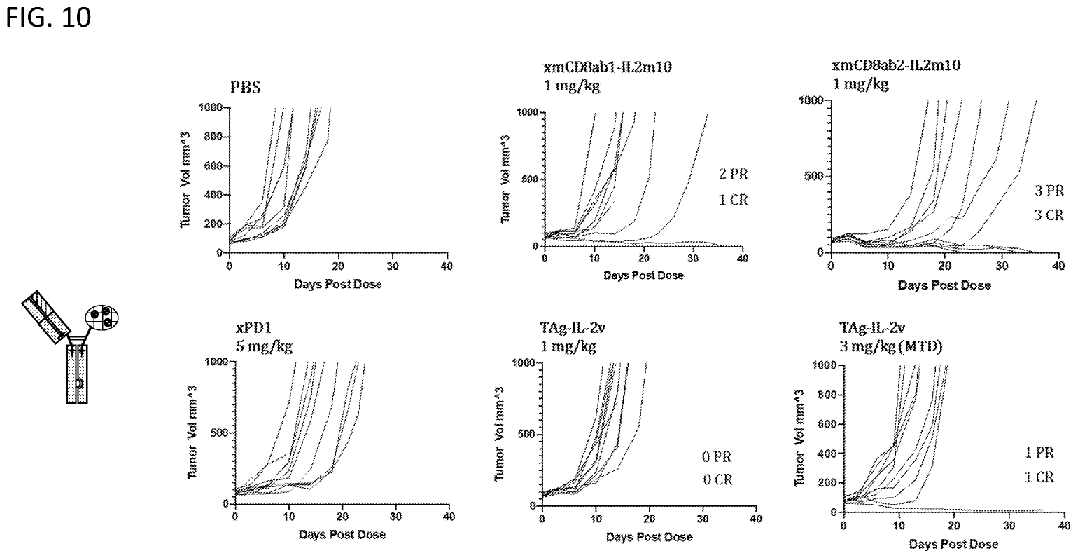

The efficacy of the CD8-IL-2 and TAg-IL-2v in combination with anti-PD1 was tested in a B16 tumor model. IL-2m10 mutein fused to one of 2 different CD8 antibodies, xmCD8ab1 (2.43 clone) and xmCD8ab2 (YTS1567.7 clone) in format C, were dosed as indicated in mice implanted with B16 tumors. As shown in FIG. 10, anti-PD1 antibody only had a modest effect in this model, with delayed tumor growth but no complete responders or cured mice. CD8-IL2 muteins induced more complete tumor regressions than TAg-IL-2v in a single dose regimen when co-administered with anti-PD1. The CD8ab2 fusion induced more complete tumor regressions than CD8ab1 fusion.

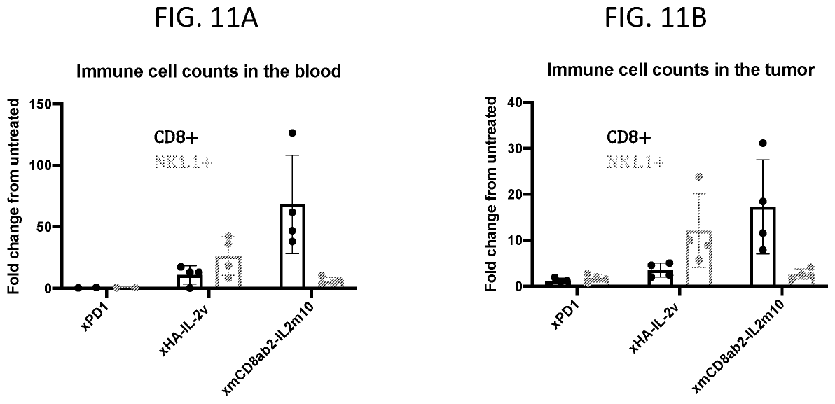

An effect on CD8+ T cell accumulation in the blood and tumor upon treatment with CD8-IL-2 fusions was tested using a B16 tumor model. IL-2 fusions at 1 mg/kg together with 5 mg/kg of xPD1 were dosed into mice implanted with B16 tumor cells. Levels of CD8+ T cells were measured in both tumors and blood samples by flow cytometry. As shown in FIGS. 11A & 11B, CD8-IL2 induced more CD8 T cell expansion in the blood (FIG. 11A) than TAg-IL-2v while TAg-IL-2v induced more NK cell expansion. Similarly, a substantial expansion of CD8+ T cells in the tumor (FIG. 11B) was observed in mice dosed with CD8-IL2 (˜17×) while only ˜3× CD8+ T cell expansion was observed in the presence of TAg-IL-2v.

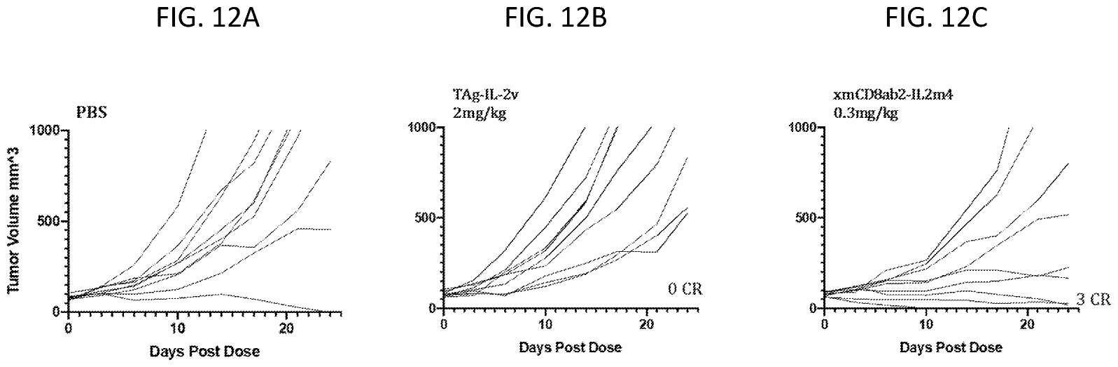

To compare the performance of CD8-IL-2 and TAg-IL-2v, tumor regression was assessed in mice implanted with CT26 tumor cells were treated with either CD8-IL-2 alone or TAg-IL-2v alone. As shown in FIGS. 12A-12C, the IL-2m4 mutein fused to xmCD8ab2 in format C induced more complete and partial tumor regressions than TAg-IL-2v in the CT26 colon tumor model when both drugs were administered as a single agent.

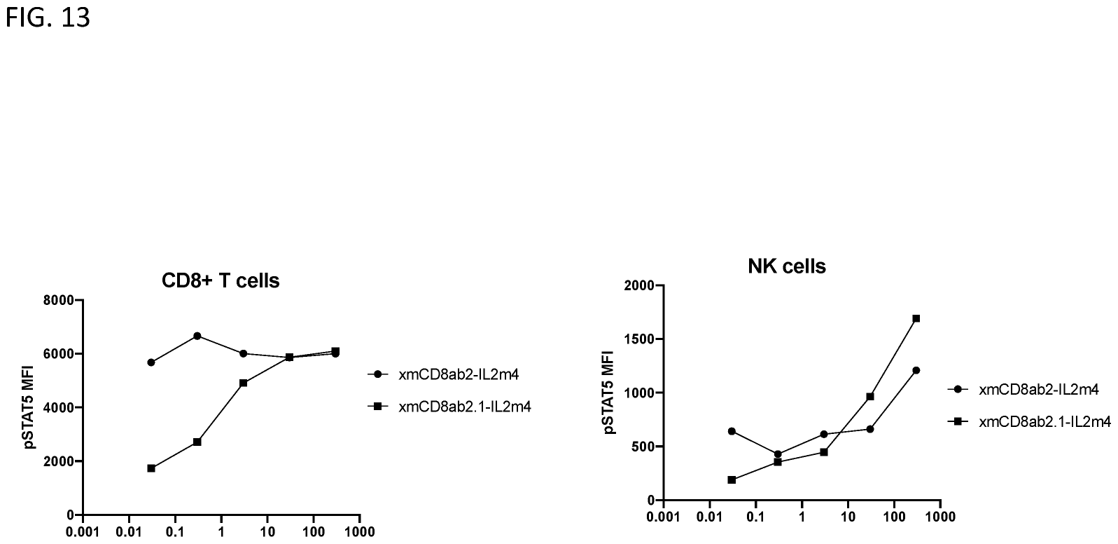

The impact of CD8 antibody affinity on potency was tested for the fusion with IL-2m4. For this experiment, IL-2 mutein IL-2m4 was fused to either xmCD8ab2 or xmCD8ab2.1 in format C. xmCD8ab2.1 is a lower affinity version of xmCD8ab2. A STAT5 assay on mouse splenocytes was performed as described in Example 1. As shown in FIG. 13, the xmCD8ab2.1-IL-2m4 fusion had lower potency and lower selectivity for CD8+ T cells over NK cells as compared to xmCD8ab2-IL-2m4.

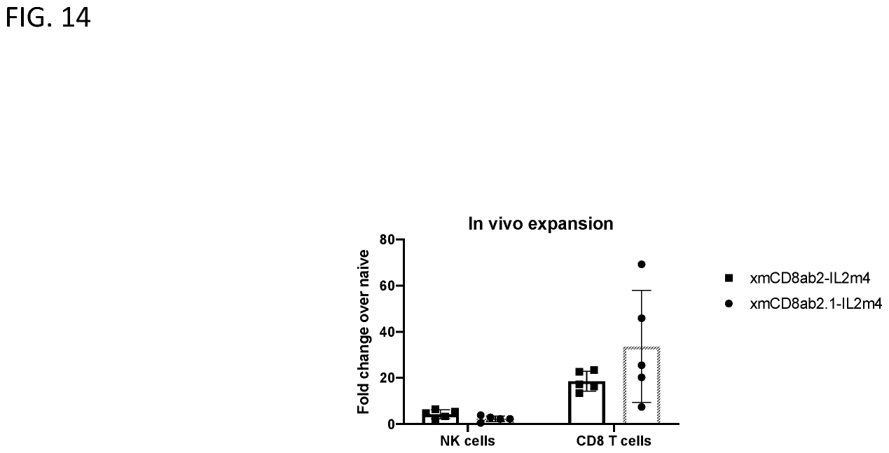

The xmCD8ab2-IL2m4 and xmCD8ab2.1-IL2m4 were also tested for their ability to expand CD8+ T cells in vivo. Blood was collected from naïve B6 mice treated with the indicated compounds at 1 mg/kg. Levels of NK cells and CD8+ T cells were determined by flow cytometry. As shown in FIG. 14, both fusions induced higher in vivo expansion of CD8 T cells than NK cells.

### Example 5: Characterization of Anti-CD8:IL-2 Mutein Fusion Proteins with Decreased Binding to IL-2Rβ and IL-2Rγ

This example describes the characterization of xmCD8-IL-2 muteins with decreased binding to IL-2Rβ/γ.

### Methods

### STAT5 Assay

Splenocytes from B6 mice bearing B16 tumors were incubated with indicated protein for 30 min in RPMI media together with anti-CD49b staining antibody after which cells were stained for cell surface markers (CD3, CD4, CD8, CD25) and for intracellular STAT5, according to the protocol in Example 1. Data was represented as mean fluorescence intensity (MFI) for STAT5 in indicated cell subsets.

### Ki67 Assay

Splenocytes from B6 mice bearing B16 tumors were incubated with indicated protein for 5 days in complete RPMI media after which cells were stained for cell surface markers (CD3, CD4, CD8, CD25, NK1.1) and for intracellular marker of proliferation, Ki67, a downstream signaling event from IL-2Rβ/γ and STAT5 according to the protocol in Example 1.

Results

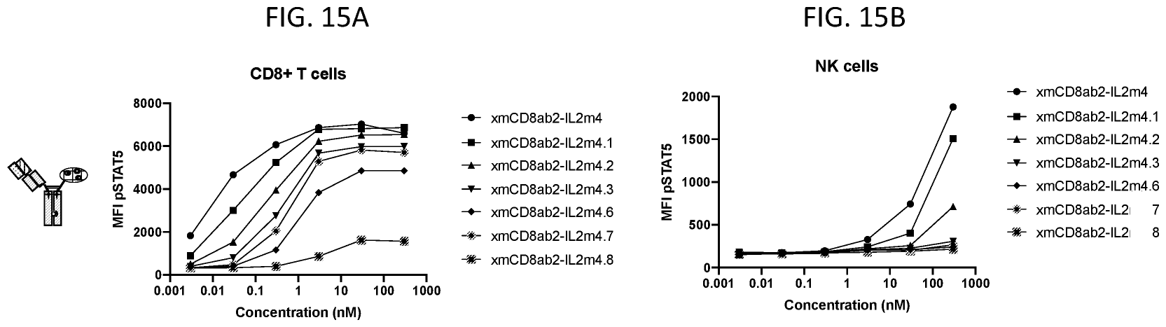

Given that active doses of xmCD8ab2-IL2m4 induced detectable NK cell expansion, additional mutations in IL2m4 were introduced in order to further decrease its activity on IL2R+ cells, including NK cells. Mutations were selected that were at the interaction surface of IL-2Rβ/γ with IL-2 (predicted to reduce binding to IL-2Rbg). IL-2 muteins with selected mutations that disrupted binding to IL-2R were fused to xmCD8ab2 antibody in format C and tested in STAT5 assay on mouse splenocytes. Table 2 and FIGS. 15A & 15B show the results of these experiments. Table 2 shows the list of mutations.

TABLE 2
STAT5 assay of IL-2 mutein sequences fused to higher
affinity CD8 antibodies.

| IL-2 mutein fused to CD8 | | | |
|---|---|---|---|
| antibody xmCD8ab2 (format C) | | STAT5 assay (MFI STAT5) | |
| IL-2 | | EC₅₀ | EC₅₀ |
| mutein | Mutations | (CD8) | (NK) |
| IL-2m4 | R38E, F42A | 0.0184 | 86.7 |
| IL-2m4.1 | R38E, F42A, I92A | 0.0648 | >100 |
| IL-2m4.2 | R38E, F42A, V91T | 0.216 | >100 |
| IL-2m4.3 | R38E, F42A, N88T | 0.388 | >100 |
| IL-2m4.4 | R38E, F42A, N88S | n.d | n.d |
| IL-2m4.5 | R38E, F42A, N88A | n.d | n.d |
| IL-2m4.6 | R38E, F42A, V91E | 1.076 | >100 |
| IL-2m4.7 | R38E, F42A, Q126S | 0.558 | >100 |
| IL-2m4.8 | R38E, F42A, Q126E | 3.94 | >100 |

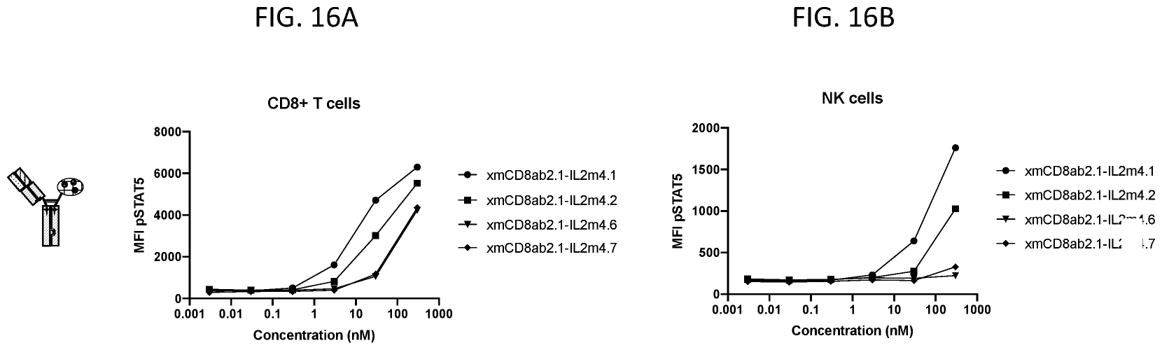

Fusions between IL-2 muteins with selected mutations and the xmCD8ab2.1 antibody in format C were also made and tested in STAT5 assay on mouse splenocytes. Table 3 and FIGS. 16A & 16B show the results of this experiment.

TABLE 3
STAT5 assay of sequences of IL-2 muteins fused to lower
affinity CD8 antibodies.

| IL-2 mutein fused to CD8 | | | |
|---|---|---|---|
| antibody xmCD8ab2 (foraat C) | | STAT5 assay (MFI STAT5) | |
| IL-2 | | EC₅₀ | EC₅₀ |
| mutein | Mutations | (CD8) | (NK) |
| IL-2m4 | R38E, F42A | n.d. | n.d. |
| IL-2m4.1 | R38E, F42A, I92A | 12.4 | 105.8 |
| IL-2m4.2 | R38E, F42A, V91T | 35.9 | >100 |
| IL-2m4.3 | R38E, F42A, N88T | n.d. | n.d. |
| IL-2m4.4 | R38E, F42A, N88S | n.d | n.d |
| IL-2m4.5 | R38E, F42A, N88A | n.d | n.d |
| IL-2m4.6 | R38E, F42A, V91E | 320.4 | >100 |
| IL-2m4.7 | R38E, F42A, Q126S | 210.5 | >100 |
| IL-2m4.8 | R38E, F42A, Q126E | n.d. | n.d. |

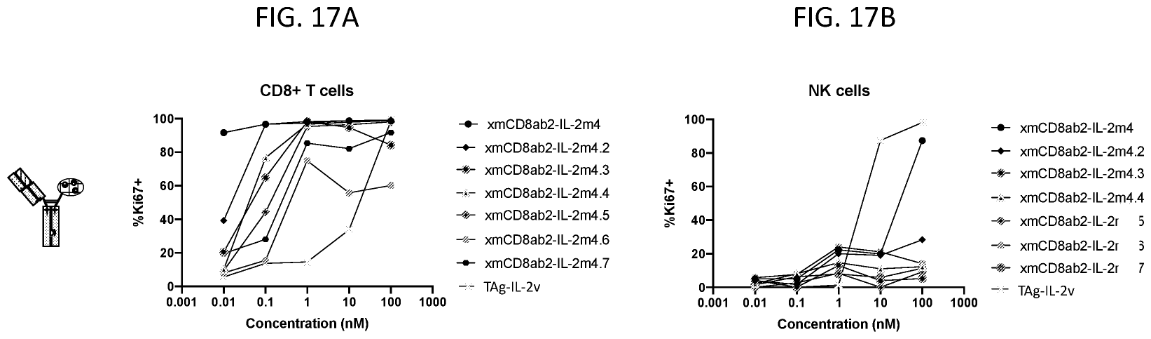

The IL-2 muteins with selected mutations were fused to xmCD8ab2 antibody in format C and tested in Ki67 assay on mouse splenocytes. Ki67 is an intracellular marker of proliferation and represents a downstream signaling event of IL-2Rβ/γ and STAT5. Table 4 and FIGS. 17A & 17B summarize the results of these experiments. TAg-2v fusion in format B was included as a reference.

TABLE 4
Ki67 assay for sequences of IL-2 muteins fused to
xmCD8ab2 anti-CD8 antibody.

| IL-2 mutein fused to CD8 antibody | | Ki67 assay (% positive) | |
|---|---|---|---|
| xmCD8ab2 (format C) | | EC₅₀ | EC₅₀ |
| IL-2 mutein | Mutations | (CD8) | (NK) |
| IL-2m4 | R38E, F42A | >0.01 | 25.1 |
| IL-2m4.1 | R38E, F42A, I92A | n.d. | n.d. |
| IL-2m4.2 | R38E, F42A, V91T | 0.0199 | >100 |
| IL-2m4.3 | R38E, F42A, N88T | 0.0414 | >100 |
| IL-2m4.4 | R38E, F42A, N88S | 0.0197 | >100 |
| IL-2m4.5 | R38E, F42A, N88A | 0.117 | >100 |
| IL-2m4.6 | R38E, F42A, V91E | 0.159 | >100 |
| IL-2m4.7 | R38E, F42A, Q126S | 0.245 | >100 |
| IL-2m4.8 | R38E, F42A, Q126E | n.d. | n.d. |
| TAg-IL-2v | F42A, Y45A, L72G* | 43.1 | 4.11 |

*IL-2v was fused to TAg antibody in format B

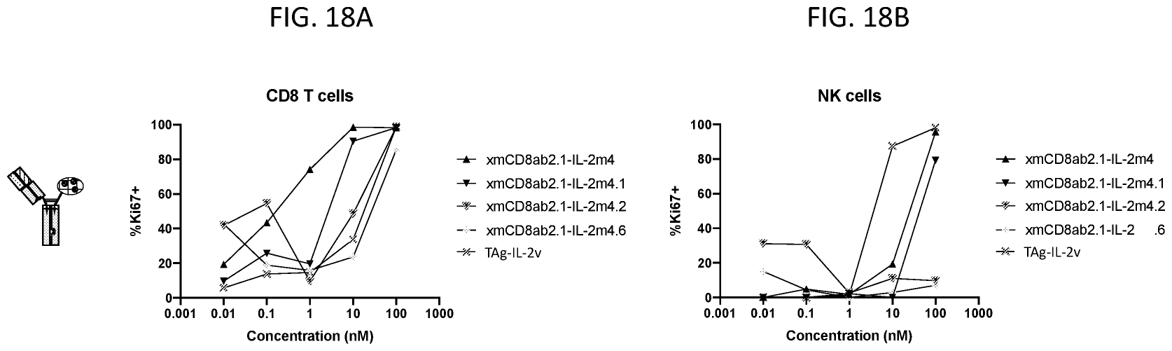

IL-2 muteins with selected mutations listed in the table below were fused to xmCD8ab2.1 antibody in format C and tested in Ki67 assay on mouse splenocytes. Table 5 and FIGS. 18A & 18B show the results of these experiments.

TABLE 5
Ki67 assay of preferred sequences of IL-2 muteins fused
to xmCD8ab2.1 anti-CD8 antibody.

| IL-2 mutein fused to CD8 antibody | | Ki67 assay (% positive) | |
|---|---|---|---|
| xmCD8ab2.1 (format C) | | EC₅₀ | EC₅₀ |
| IL-2 mutein | Mutations | (CD8) | (NK) |
| IL-2m4 | R38E, F42A | 0.15 | 23.5 |
| IL-2m4.1 | R38E, F42A, I92A | 2.28 | 47.3 |
| IL-2m4.2 | R38E, F42A, V91T | 8.88 | >100 |
| IL-2m4.3 | R38E, F42A, N88T | n.d | n.d |
| IL-2m4.4 | R38E, F42A, N88S | n.d | n.d |
| IL-2m4.5 | R38E, F42A, N88A | n.d | n.d |
| IL-2m4.6 | R38E, F42A, V91E | 23.7 | >100 |
| IL-2m4.7 | R38E, F42A, Q126S | n.d | n.d |
| IL-2m4.8 | R38E, F42A, Q126E | n.d | n.d |

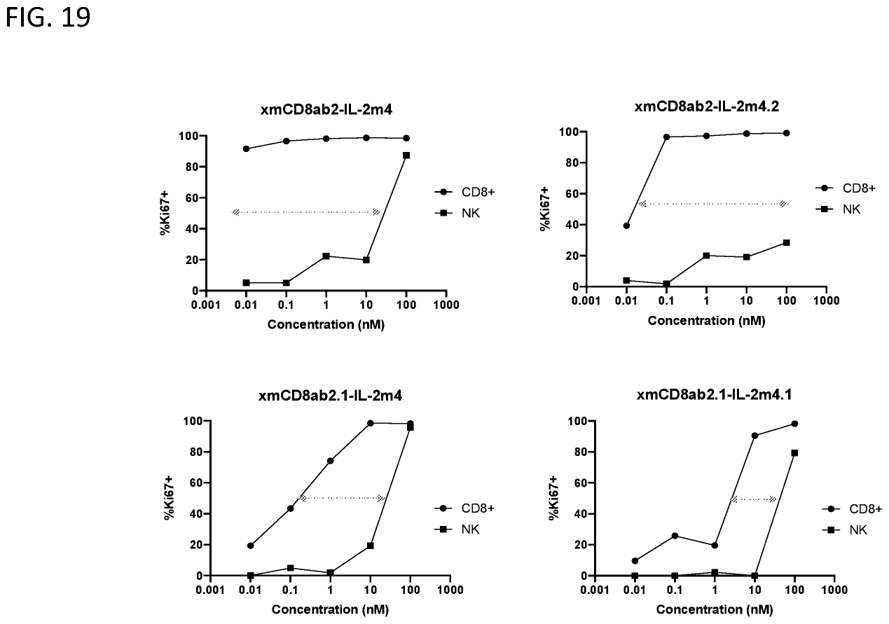

The potencies on CD8 T cells and NK cells for representative molecules are summarized in FIG. 19. Generated CD8-IL2 fusions have a range of selectivity for CD8 T cells over NK cells with xmCD8ab2-IL2m4 and xmCD8ab2-IL2m4.2 being the highest (>1000×), followed by xmCD8ab2.1-IL2m4 (˜50-100×) and xmCD8ab2.1-IL2m4.2 the lowest (˜10×). The m4 mutein fusion has lowered binding to IL-2Rα, while the m4.1 and m4.2 muteins have contain additional mutations that lower binding to IL-2Rγγ.

### Example 6: Testing Effects of CD8:IL2 Fusion Proteins in Combination with Anti-PD-1 in a B16 Tumor Model

### Results

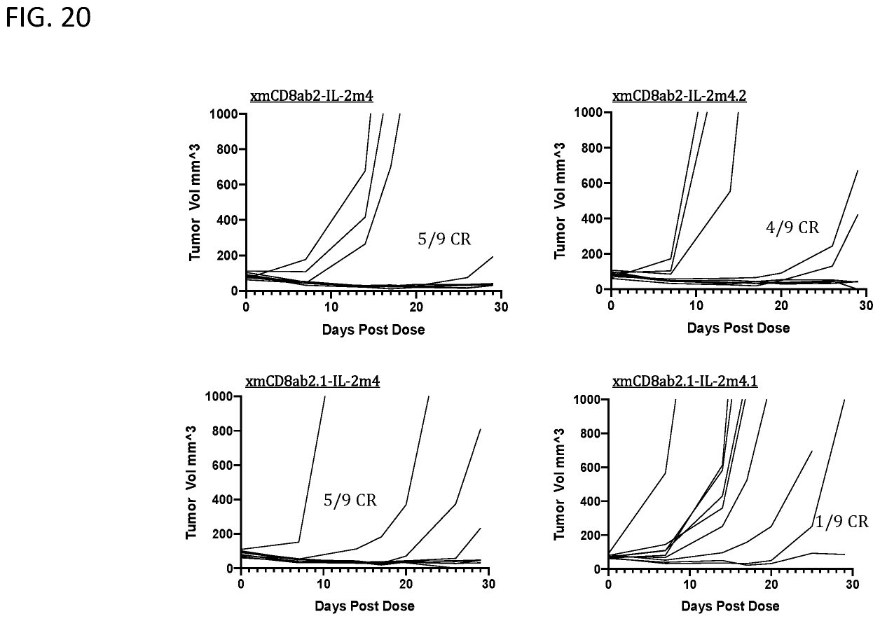

Four representative CD8-IL2 fusions with varying degrees of selectivity for CD8 T cells over NK cells were tested in the B16 tumor model as described in Example 4. All mice were dosed with 1 mg/kg of the indicated fusions together with 5 mg/kg of anti-PD1. As shown in FIG. 20, CD8-IL-2 performed better than TAg-IL-2v (see FIG. 10) at a lower dose. CD8-IL2 fusion with the lowest selectivity for CD8 T cells had the least efficacy in the B16 model, approaching that observed for TAg-IL-2v (FIG. 10). Selectivity of >10× was required for best efficacy and >40% tumor free mice.

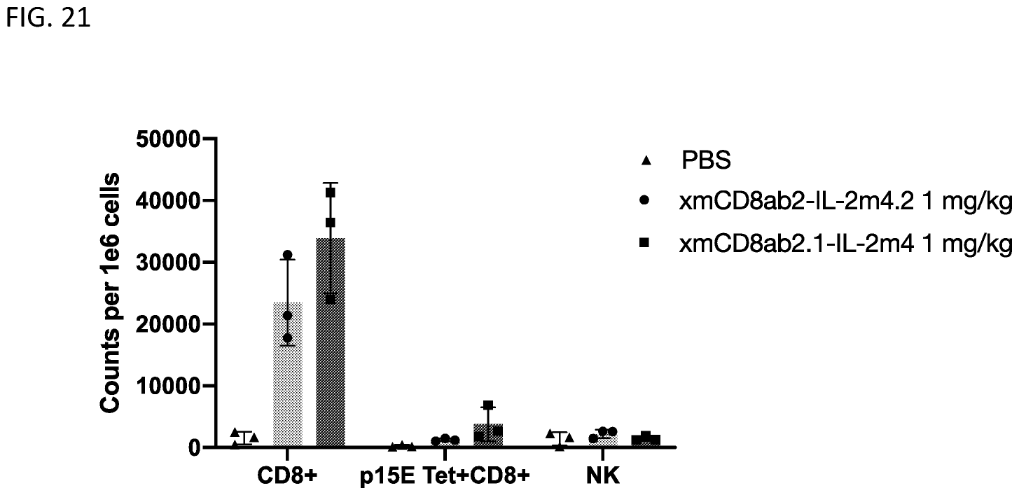

The performance of CD8-IL-2 was further tested by analyzing the expansion of tumor-antigen specific T cells upon treatment. B6 mice were injected with B16 tumor cells and tumors allowed to grow to 200-250 mm³ before they were dosed with indicated IL-2 fusions at 1 mg/kg together with 5 mg/kg of xPD1. Tumors were removed day 5 post dose, digested to single cells and profiled by flow cytometry to detect CD8+ T cells and NK cells (NK1.1+CD3−). Cells were also stained with p15E tetramer (TB-M507-2, MBL) according to manufacturer's protocol to detect T cells that recognize p15E tumor antigen. As shown in FIG. 21, both xmCD8ab2-IL2m4.2 and xmCD8ab2.1-IL2m4 induced >15× expansion of total intratumoral CD8+ T cells and 5-17× expansion in p15E tumor antigen-specific T cells with low to no expansion of NK cells.

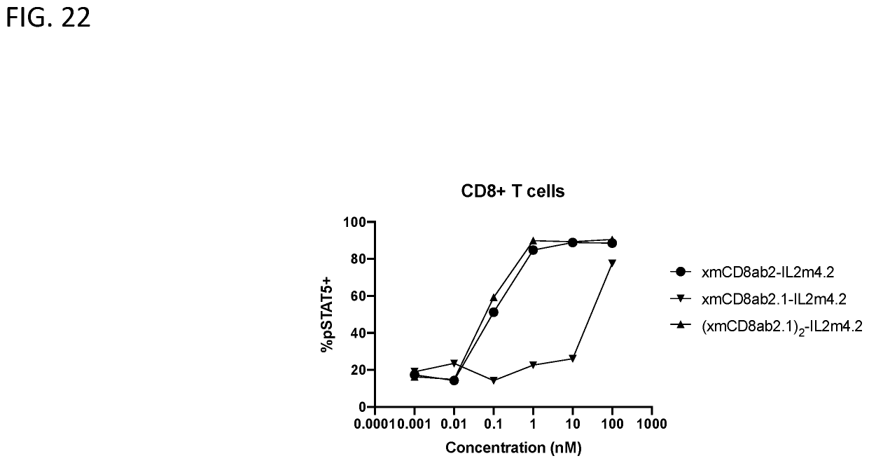

A STAT5 assay was performed to compare the potency of a bivalent low affinity fusion and a high affinity monovalent fusion. Splenocytes from B6 mice bearing B16 tumors were incubated with a fusion protein for 30 min in RPMI media after which cells were stained for cell surface markers (CD3, CD4, CD8, CD25) and for intracellular STAT5, according to the protocol in Example 1. As shown in FIG. 22, the bivalent low affinity fusion had similar potency as a high affinity monovalent fusion. IL-2m4.2 fusion fused to high affinity xmCD8ab2 antibody (in format C) or to bivalent xmCD8ab2.1 antibody (in format A) had similar potencies on CD8+ T cells and much greater potency than monovalent xmCD8ab2.1-IL2m4 (format C) fusion.

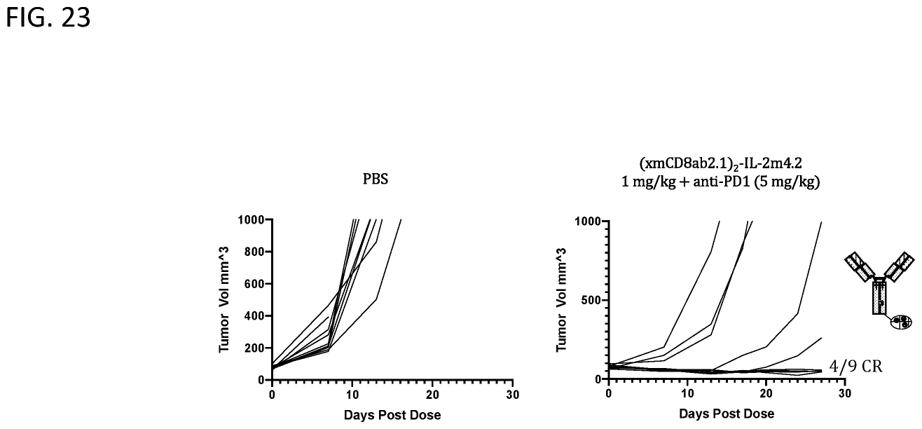

To further test the affinity of a bivalent C-terminal format (format A), bivalent xmCD8ab2.1 antibody (in format A) was tested in the B16 tumor model as described in Example 4. Mice were dosed with PBS as control or 1 mg/kg of the indicated fusion together with 5 mg/kg of anti-PD1 (9 mice per group). As shown in FIG. 23, IL-2m4.2 fusion fused to high affinity xmCD8ab2 antibody (in format C) or to bivalent xmCD8ab2.1 antibody (in format A) had similar in vivo efficacy to IL-2m4.2 fusion fused to high affinity xmCD8ab2 antibody (in format C) (see FIG. 20). Thus, bivalent C-terminal format (format A) is also very efficacious.

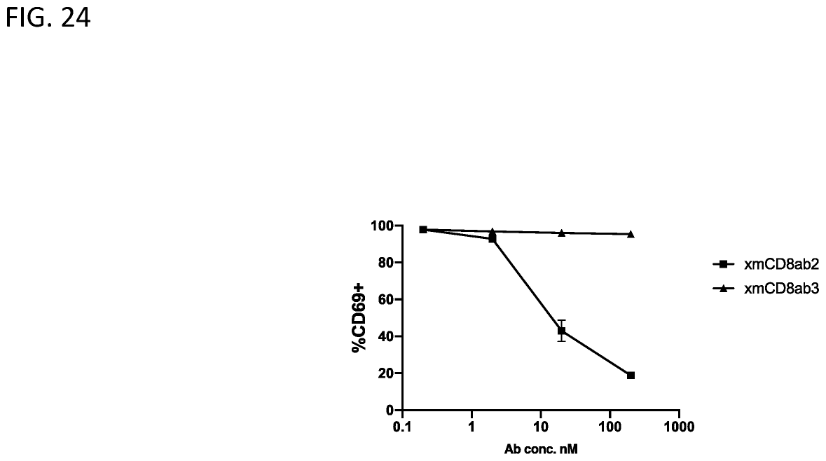

Blocking of CD8 T cell activation by CD8 antibodies was also tested. CD8+ T cells were purified from splenocytes from OT-I mice and co-cultured with EL-4-OVA line (ATCC), at 100,000 cells each for 24 hr. Cells were analyzed for upregulation of activation markers such as CD25 and CD69 by cell surface staining and flow cytometry as described in Example 3. As shown in FIG. 24, certain CD8 antibodies did not block CD8 T cell activation. The xmCD8ab3 antibody did not block CD8 T cell activation even at 200 nM concentration. The xmCD8ab3 antibody was bivalent.

Blocking of CD8 T cell activation by CD8 antibodies was also tested. CD8+ T cells were purified from splenocytes from OT-I mice and co-cultured with EL-4-OVA line (ATCC), at 100,000 cells each for 24 hr. Cells were analyzed for upregulation of activation markers such as CD25 and CD69 by cell surface staining and flow cytometry as described in Example 3. As shown in FIG. 24, certain CD8 antibodies did not block CD8 T cell activation. The xmCD8ab3 antibody did not block CD8 T cell activation even at 200 nM concentration. The xmCD8ab3 antibody was bivalent.

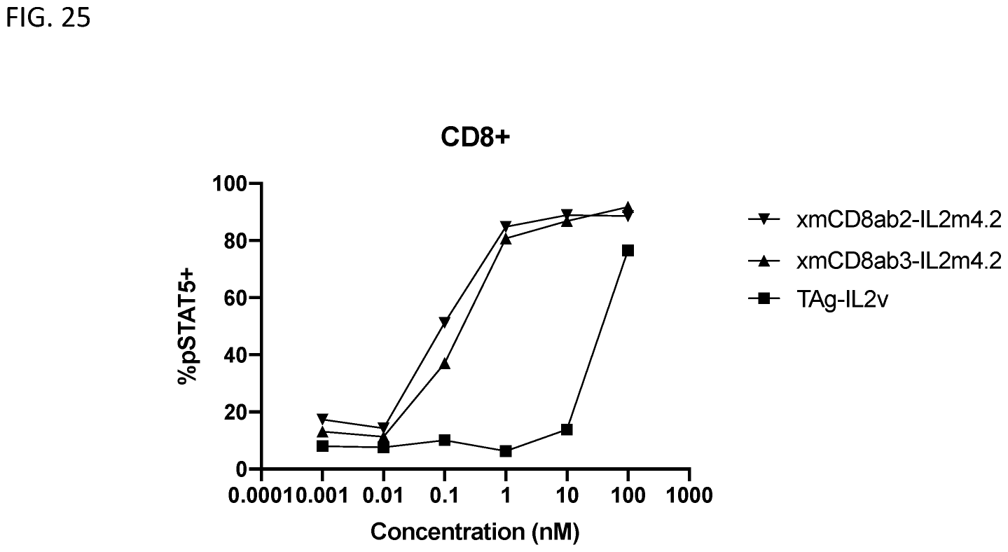

To compare the in vivo potency of the xmCD8ab2 and xmCD8ab3 fusions, splenocytes from B6 mice bearing B16 tumors were incubated with indicated protein for 30 min in RPMI media after which cells were stained for cell surface markers (CD3, CD4, CD8, CD25, NK1.1) and for intracellular STAT5, according to the protocol in Example 1. As shown in FIG. 25, xmCD8ab2-IL2m4.2 and xmCD8ab3-IL2m4.2 exhibited similar activity on CD8+ T cells in vitro.

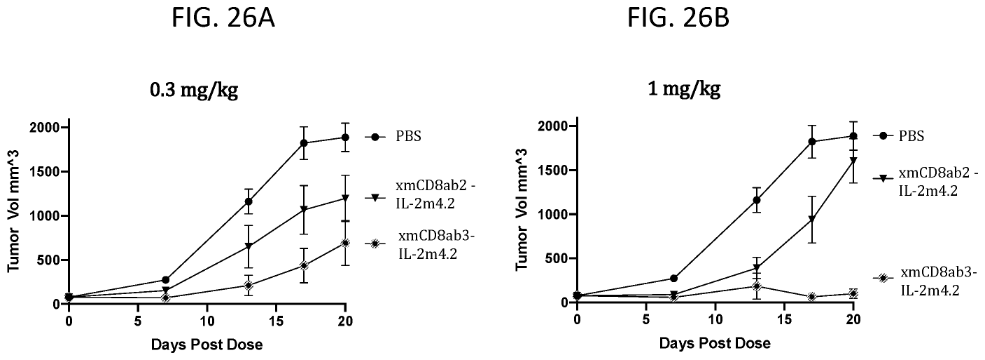

IL-2m4.2 fused to MHC non-blocking antibodies were also tested in a B16 tumor model as described in Example 4. Mice were dosed with PBS, 0.3 mg/kg (FIG. 26A) or 1 mg/kg (FIG. 26B) of the indicated fusions together with 5 mg/kg of anti-PD1 (9 per group). As shown in FIGS. 26A & 26B, the MHC non-blocking antibody was more optimal in vivo. IL-2m4.2 fusion fused to MHC non-blocking xmCD8ab3 antibody (in format C) was much more efficacious than IL-2m4.2 fusion fused to MHC blocking xmCD8ab2 antibody (in format C).

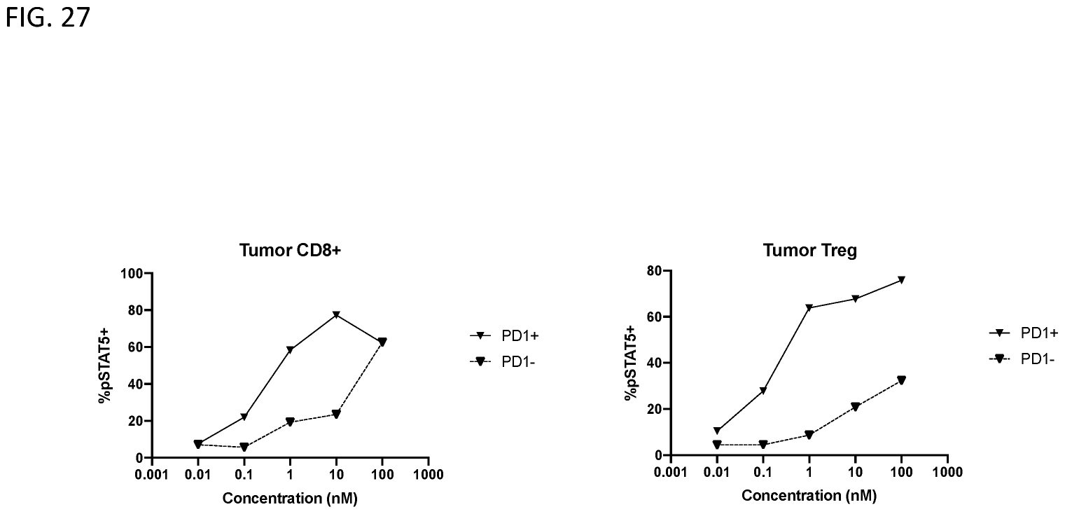

Preferential targeting of targeting of PD1+ T cells, both CD8+ and Treg by fusions of IL-2 muteins was also tested. B16 tumors 300-600 mm³ in size were removed from mice and digested to single cells. CD45+ cells were purified (Miltenyi's LS columns according to manufacturer's protocol) and stimulated with indicated fusion proteins for 30 min. Cells were stained for cell surface markers (CD3, CD4, CD8, CD25, CD49b, and PD1) and for intracellular phospho-STAT5. As shown in FIG. 27, fusion of IL-2 mutein IL2m10 with anti-PD1 antibody preferentially targeted PD1+ T cells over PD1− T cells; however both CD8+PD1+ T cells and CD4+CD25+PD1+ Treg cells were targeted.

### Example 7: Impact of IL-2 Mutations on Activity and Selectivity of CD8-IL-2 Fusions in hPBMCs

This example describes the impact of IL-2 mutations on the activity and selectivity of CD8-IL-2 fusions in hPBMCs.

### Results

To characterize the impact of IL-2 mutations on the activity of CD8-IL-2 fusions in hPBMCs, indicated IL-2 muteins were fused to xmCD8ab1 antibody and tested in STAT5 assay on hPBMCs. The xmCD8ab1 antibody does not recognize human CD8. The obtained data was used to rank each mutation according to its effect on STAT5 activity. Tables 6, 7 and 8 summarize the results of this experiment. Tregs are shown as representative examples of cells expressing IL-2Rγγ.

Table 6 depicts activity of IL-2 muteins IL-2 m1 to IL-2m10 fused to xmCD8ab1 on human Tregs as it compares to IL-2v fused to control xHA antibody, all in format B. Table 6 shows that IL-2 muteins IL-2 m1 to IL-2m10 fused to xmCD8ab1 all had significantly reduced activity on Tregs, thereby significantly reduced binding to IL-2Rα, compared to wild-type IL-2, and comparable to IL-2v which has no binding to IL-2Rα(Klein et al, Oncoimmunol. 2017; 6 (3); e1277306).

TABLE 6
Impact of IL-2 mutations on activity in hPBMCs

| xmCD8ab1 fusion | | | | | | |
|---|---|---|---|---|---|---|
| (format B) | | % STAT5+ in human Tregs | | | | |
| IL-2 mutein | Mutation | 100 nM | 10 nM | 1 nM | 0.1 nM | EC50 |
| IL-2m1 | F42A | 93.3 | 90.6 | 87.7 | 44.4 | <0.1 |
| IL-2m2 | R38A, F42A | 91.9 | 91.3 | 66.1 | 15 | 0.37 |
| IL-2m3 | R38D, F42A | 76 | 85.7 | 45 | 4.24 | 0.66 |
| IL-2m4 | R38E, F42A | 89.4 | 84.6 | 34.8 | 6.02 | 1.54 |
| IL-2m5 | F42A, E62Q | 87.4 | 85.5 | 41.4 | 6.46 | 1.09 |
| IL-2m6 | F42A, E68A | 87 | 80.3 | 55.4 | 12.2 | 0.56 |
| IL-2m7 | F42A, E68Q | 84.3 | 85.3 | 83 | 58.4 | <0.1 |
| IL-2m8 | F42A, E68K | 73.4 | 75.9 | 27.2 | 13 | 1.95 |
| IL-2m9 | F42A, E68R | 89.3 | 75 | 28.1 | 8.64 | 2.64 |
| IL-2m10 | R38A, F42K | 94.1 | 88.1 | 39.1 | 3.11 | 1.28 |
| IL-2v* | F42A, Y45A, | 92.8 | 84.8 | 28.9 | 8.77 | 2.27 |
| | L72G | | | | | |

*IL-2v was fused to control xHA antibody.

Table 7 depicts the activity of IL-2 muteins IL-2m10.1 to IL-2m10.11 fused to xmCD8ab1 on human Tregs as compared to IL-2m10 fused to xmCD8ab1, all in format B. Table 7 shows that IL-2m10.1 to IL-2m10.11 fused to xmCD8ab1 all have significantly reduced activity on Tregs compared to IL-2m10. IL-2m10 has reduced to no activity on IL2Ra, therefore additional mutations present in IL-2m10.1 to IL-2m10.11 reduced activity of the molecules by decreasing the binding to IL-2Rγγ.

TABLE 7
Impact of IL-2 mutations on activity
of CD8-IL-2 fusions in hPBMCs

| xmCD8ab1 fusion | | | | | | |
|---|---|---|---|---|---|---|
| (format B) | | | | | | |
| | Added | % STAT5+ in human Tregs | | | | |
| IL-2 mutein | mutation | 100 nM | 10 nM | 1 nM | 0.1 nM | EC50 |
| IL-2m10 | — | 94.1 | 88.1 | 39.1 | 3.11 | 1.28 |
| IL-2m10.1 | T123A | 91.3 | 59.4 | 7.56 | 7.95 | 7.72 |
| IL-2m10.2 | T123E | 92.5 | 82.6 | 20.8 | 8.28 | 2.97 |
| IL-2m10.3 | T123K | 92.1 | 64.6 | 10.3 | 6.88 | 6.11 |
| IL-2m10.4 | T123Q | 85.1 | 68.2 | 22.2 | 10.6 | 3.80 |
| IL-2m10.5 | S127A | 90.1 | 79.7 | 18.8 | 11.1 | 3.37 |
| IL-2m10.6 | S127E | 91.3 | 71.3 | 17.6 | 10.2 | 4.52 |
| IL-2m10.7 | S127K | 87.3 | 70/9 | 19.8 | 12.7 | 4.16 |
| IL-2m10.8 | S127Q | 85.3 | 76.2 | 19.6 | 10.7 | 3.18 |
| IL-2m10.9 | Q126S | 85.8 | 27.6 | 3.61 | 4.25 | 35.2 |
| IL-2m10.10 | Q126T | 76.8 | 24.3 | 3.21 | 5.7 | 39.7 |
| IL-2m10.11 | Q126E | 43.5 | 17.1 | 13.1 | 8.12 | 64.7 |

Table 8 depicts the activity of IL-2 muteins IL-2m4.1 to IL-2m4.6 and IL-2m4.9 to IL-2m4.24 fused to xmCD8ab1 on human Tregs as compared to IL-2m4 fused to xmCD8ab1, all in format B. IL-2m4 has reduced to no activity on IL2Rα, therefore additional mutations present in the IL-2 mutein fusion proteins shown in Table 8 reduced activity of the molecules by decreasing the binding to IL-2Rγγ. Decrease in activity on Tregs and therefore in binding to IL-2Rbg was observed for all of the IL-2 mutein fusions except for IL-2m4.9, IL-2m4.10, IL-2m4.12, and IL-2m4.16.

TABLE 8
Impact of IL-2 mutations on activity
of CD8-IL-2 fusions in hPBMCs

| xmCD8ab1 fusion | | | | | | |
|---|---|---|---|---|---|---|
| (format C) | | | | | | |
| | Added | % STAT5+ in human Tregs | | | | |
| IL-2 mutein | mutation | 300 nM | 30 nM | 3 nM | 0.3 nM | EC50 |
| IL-2m4* | — | 94.8 | 78.3 | 24.8 | 4.57 | 2.822 |
| IL-2m4.9 | E15A | 94.3 | 83.1 | 52.9 | 11.2 | 2.396 |
| IL-2m4.10 | R81E | 94.1 | 94.6 | 73.7 | 47.4 | 1.899 |
| IL-2m4.11 | E95S | 93.2 | 77.4 | 22.3 | 8.52 | 10.05 |
| IL-2m4.12 | R81Q | 92.7 | 90.2 | 53.4 | 26.7 | 3.652 |
| IL-2m4.13 | E95A | 92.5 | 79.5 | 25 | 6.99 | 8.21 |
| IL-2m4.14 | M23A | 90 | n.d. | 18.2 | 3.63 | 13.76 |
| IL-2m4.15 | S87A | 90 | 75.2 | 26 | 3.54 | 7.338 |
| IL-2m4.16 | E15Q | 87.5 | 85.6 | 47.9 | 15.3 | 3.026 |
| IL-2m4.17 | V91A | 83.3 | 59.9 | 11 | 6.33 | 16.88 |
| IL-2m4.2 | V91T | 81.4 | 36.2 | 6.98 | 4.29 | 54.24 |
| IL-2m4.1 | I92A | 77.1 | 33.7 | 6.7 | 4.92 | 58.04 |
| IL-2m4.6 | V91E | 60.6 | 13.8 | 7.48 | 2.5 | ~150 |
| IL-2m4.18 | E95R | 58.7 | 13.7 | 3.22 | 3.47 | ~150 |
| IL-2m4.4 | N88S | 29.1 | 9.33 | 4.15 | 5.34 | >250 |
| IL-2m4.19 | H16E | 21.4 | 2.76 | 2.62 | 2.43 | >250 |
| IL-2m4.5 | N88A | 18.4 | 2.62 | 4.55 | 3.37 | >250 |
| IL-2m4.20 | H16D | 15.3 | 3.72 | 2.4 | 3.27 | >250 |
| IL-2m4.3 | N88T | 4.07 | 1.98 | 2.55 | 3.65 | >1000 |
| IL-2m4.21 | D20N | 4.0 | 3.8 | 4.48 | 3.14 | >1000 |
| IL-2m4.22 | N88R | 2.65 | 3 | 3.44 | 2.75 | >1000 |
| IL-2m4.23 | N88I | 2.38 | 2.17 | 3.24 | 3.82 | >1000 |
| IL-2m4.24 | N88E | 1.79 | 3.04 | 1.64 | 4.93 | >1000 |

The selective stimulation of human CD8+ T cells by the CD8-IL-2 mutein fusions was also tested. Indicated IL-2 muteins were fused to previously published anti-human CD8 antibody clone OKT8 (xhCD8ab) in format C and tested in STAT5 assay on hPBMCs. Tables 9 and 10 summarize the results of this experiment. CD8-IL2 mutein fusions selectively and potently stimulated human CD8 T cells. IL-2m4 had reduced to no activity on IL2Ra, therefore additional mutations present in the IL-2 mutein fusion proteins shown in Table 9 reduced activity of the molecules by decreasing the binding to IL-2Rbg. Decrease in activity on Tregs and therefore in binding to IL-2Rbg was observed for all of the IL-2 mutein fusions except for IL-2m4.26, IL-2m4.27, IL-2m4.28 and IL-2m4.29.

Certain mutations were not useful because they resulted in very weak CD8 T cell activation (i.e. IL-2 mutein containing N88E mutation only very weakly activated CD8 T cells in the context of the fusion with xhCD8ab while N88A/S/T generated CD8-IL2 molecules with potent activity on CD8 T cells).

TABLE 9
Stimulation of human Tregs by CD8-IL-2 molecules.

| xhCD8ab fusion | | | | | | | |
|---|---|---|---|---|---|---|---|
| (format C) | | % STAT5+ in human Tregs | | | | | |
| IL-2 mutein | Mut. | 100 nM | 10 nM | 1 nM | 0.1 nM | 0.01 nM | EC50 |
| IL-2m4.26 | E15R | 96.4 | 98.1 | 83.2 | 22.4 | 4.25 | 0.262 |
| IL-2m4.27 | E15K | 98 | 97.8 | 59 | 9.76 | 4.69 | 0.748 |
| IL-2m4.28 | M23E | 97.6 | 93.3 | 53.4 | 6.73 | 1.85 | 0.866 |
| IL-2m4.29 | M23Q | 96.9 | 93.2 | 50.2 | 7.21 | 7.77 | 1.083 |
| IL-2m4.30 | M23R | 96.5 | 63.6 | 16.9 | 6.5 | 2.32 | 6.438 |
| IL-2m4.31 | D87K | 94.2 | 63.4 | 13.3 | 4.18 | 3.35 | 6.376 |
| IL-2m4.32 | M23K | 99 | 63.1 | 17.2 | 3.81 | 2.67 | 6.844 |
| IL-2m4.33 | D84L | 93.2 | 36.5 | 6.02 | 2.79 | 3.39 | 22.96 |
| IL-2m4.34 | D84N | 92 | 32.7 | 11.1 | 3.5 | 1.9 | 28.44 |
| IL-2m4.35 | D84V | 91.4 | 32.4 | 6.12 | 5.71 | 6.96 | 32.63 |
| IL-2m4.6 | V91E | 84 | 24.6 | 7.29 | 5.79 | 3.95 | 49.83 |
| IL-2m4.36 | D84H | 82.9 | 17.9 | 5.75 | 9.15 | 6.37 | 196.9 |
| IL-2m4.37 | D84Y | 78.6 | 18.1 | 4.51 | 3.12 | 5.93 | 93.42 |
| IL-2m4.38 | Q126S | 79.8 | 33.1 | 15.8 | 9.04 | 7.54 | 26.71 |
| IL-2m4.39 | Q126E | 41.7 | 8.68 | 6.38 | 3.59 | 2.81 | >250 |
| IL-2m4.4 | N88S | 37.9 | 11.6 | 6.51 | 7.69 | 8.24 | >250 |
| IL-2m4.5 | N88A | 35.7 | 8.61 | 3.86 | 3.7 | 10.9 | >250 |
| IL-2m4.20 | H16D | 30.7 | 6.99 | 4.7 | 1.94 | 4.21 | >250 |
| IL-2m4.19 | H16E | 31 | 5.23 | 1.6 | 4.04 | 2.39 | >250 |
| IL-2m4.40 | D84R | 17.9 | 3.72 | 2.14 | 2.58 | 6.54 | >1000 |
| IL-2m4.21 | N88T | 9.52 | 7.42 | 4.84 | 7.79 | 3.57 | >1000 |
| IL-2m4.41 | D84K | 10.7 | 3.16 | 5.65 | 2.81 | 2.5 | >1000 |
| IL-2m4.23 | N88R | 6.65 | 4.12 | 4.38 | 4.85 | 3.34 | >1000 |
| IL-2m4.25 | N88E | 3.41 | 3.32 | 2.55 | 3.36 | 1.84 | >1000 |

TABLE 10
Stimulation of human CD8+ T cells by CD8-IL-2 molecules.

| xhCD8ab fusion | | | | | | | |
|---|---|---|---|---|---|---|---|
| (format C) | | % STAT5 in human CD8+ T cells | | | | | |
| IL-2 mutein | Mut. | 100 nM | 10 nM | 1 nM | 0.1 nM | 0.01 nM | EC50 |
| IL-2m4.26 | E15R | 93.8 | 92.4 | 88.2 | 79.7 | 73.2 | <0.01 |
| IL-2m4.27 | E15K | 94 | 90.6 | 81.2 | 76.4 | 72.6 | <0.01 |
| IL-2m4.28 | M23E | 95.4 | 91.7 | 82.2 | 76.5 | 70.5 | <0.01 |
| IL-2m4.29 | M23Q | 93.5 | 89.2 | 76.9 | 71.6 | 67.2 | <0.01 |
| IL-2m4.30 | M23R | 91.1 | 80.6 | 76.1 | 74.1 | 65.3 | <0.01 |
| IL-2m4.31 | D87K | 90.5 | 79.7 | 72.4 | 72.5 | 62.2 | <0.01 |
| IL-2m4.32 | M23K | 92.1 | 84.1 | 77.1 | 74.3 | 59.2 | <0.01 |
| IL-2m4.33 | D84L | 89.6 | 78.3 | 76 | 73.3 | 48.9 | 0.027 |
| IL-2m4.34 | D84N | 87.8 | 76.9 | 73.6 | 75 | 44.2 | 0.007 |
| IL-2m4.35 | D84V | 88.5 | 78.7 | 75.3 | 72.5 | 42.4 | 0.020 |
| IL-2m4.6 | V91E | 85.7 | 79.2 | 75.7 | 71.9 | 49 | 0.029 |
| IL-2m4.36 | D84H | 84.9 | 76.4 | 76 | 69.9 | 34.4 | 0.016 |
| IL-2m4.37 | D84Y | 85.1 | 78.1 | 75.4 | 70.4 | 29 | 0.012 |
| IL-2m4.38 | Q126S | 84.8 | 80.5 | 78.8 | 68 | 33.1 | 0.027 |
| IL-2m4.4 | N88S | 80.3 | 75.6 | 77.4 | 56.3 | 18.1 | 0.042 |
| IL-2m4.39 | Q126E | 70.6 | 71 | 67.3 | 40 | 6.03 | 0.072 |
| IL-2m4.5 | N88A | 77.1 | 75.7 | 71.3 | 48.1 | 18.4 | 0.077 |
| IL-2m4.20 | H16D | 76.4 | 76.5 | 71.9 | 50.3 | 8.04 | 0.046 |
| IL-2m4.19 | H16E | 71.8 | 75.9 | 72.3 | 38.7 | 4.3 | 0.080 |
| IL-2m4.40 | D84R | 76.1 | 74 | 67.2 | 35.7 | 7.37 | 0.118 |
| IL-2m4.21 | N88T | 75.5 | 74.8 | 64.5 | 18.5 | 4.13 | 0.259 |
| IL-2m4.41 | D84K | 74.9 | 76.8 | 64.7 | 23.3 | 3.46 | 0.213 |
| IL-2m4.23 | N88R | 68.2 | 61.1 | 21.2 | 6.95 | 5.68 | 2.54 |
| IL-2m4.25 | N88E | 12.1 | 6.78 | 3.83 | 3.75 | 3.37 | >100 |

Tables 11 and Table 12 depict the activity of IL-2m10.12 mutein containing Q126A mutation in the context of the fusion to either control antibody (xHA) or xhCD8ab antibody. This mutation in the context of CD8-IL2 fusion decreased the activity of the CD8-IL2 fusion on human Tregs compared to that containing the IL-2m10 mutein (Tables 6 and 7) but enabled potent and selective activation of CD8+ T cells.

TABLE 11

| Fusion (format B) | | | | | | | |
|---|---|---|---|---|---|---|---|
| | | Added | % STAT5+ in human Tregs | | | | |
| Antibody | IL-2 mutein | mutation | 100 nM | 10 nM | 1 nM | 0.1 nM | EC50 |
| xHA | IL-2m10.12 | Q126A | 77.7 | 21.8 | 4.88 | 5.38 | 55.1 |
| xhCD8ab | IL-2m10.12 | Q126A | 66.6 | 19.9 | 8.39 | 6.46 | 60.8 |

TABLE 12

| Fusion (format B) | | | | | | | |
|---|---|---|---|---|---|---|---|
| | | Added | % STAT5+ in human CD8+ T cells | | | | |
| Antibody | IL-2 mutein | mutation | 100 nM | 10 nM | 1 nM | 0.1 nM | EC50 |
| xHA | IL-2m10.12 | Q126A | 19.4 | 0.79 | 0.60 | 0.67 | >100 |
| xhCD8ab | IL-2m10.12 | Q126A | 68.1 | 69.1 | 68.2 | 55.9 | <0.1 |

What is claimed is:

1. A fusion protein comprising two moieties, wherein:
i) The first moiety comprises an antibody heavy chain VH—CH1-hinge-CH2-CH3 monomer wherein VH is a variable heavy chain and CH2-CH3 is a Fc domain, an antibody light chain VL-CL wherein VL is a variable light chain and CL is a constant light chain, and a mutant IL-2 polypeptide, wherein the N-terminus of the mutant IL-2 polypeptide is fused to the C-terminus of the Fc domain via a linker;
ii) The second moiety comprises an antibody heavy chain VH—CH1-hinge-CH2-CH3 monomer and an antibody light chain VL-CL;
and wherein, both the first and second moiety bind to an epitope on one antigen selected from the following group: human CD8α, human CD8β, and human PD1.

2. A fusion protein comprising two moieties, wherein:
i) The first moiety is a polypeptide comprising an antibody hinge-CH2-CH3 monomer wherein CH2-CH3 is a Fc domain, and a mutant IL-2 polypeptide, wherein the N-terminus of the mutant IL-2 polypeptide is fused to the C-terminus end of the Fc domain via a linker;
ii) The second moiety is a polypeptide comprising an antibody heavy chain VH—CH1-hinge-CH2-CH3 monomer and an antibody light chain VL-CL;
and wherein the second moiety binds to an epitope on one antigen selected from the following group: human CD8α, human CD8β, and human PD1.

3. A fusion protein comprising two moieties, wherein:
i) The first moiety is a polypeptide comprising an antibody hinge-CH2-CH3 monomer wherein CH2-CH3 is a Fc domain, and a mutant IL-2 polypeptide, wherein the C-terminus of the mutant IL-2 polypeptide is fused to the N-terminus end of the Fc domain via a linker;
ii) The second moiety is a polypeptide comprising an antibody heavy chain VH—CH1-hinge-CH2-CH3 monomer and an antibody light chain VL-CL;
and wherein the second moiety binds to an epitope on one antigen selected from the following group: human CD8α, human CD8β, and human PD1.

4. A fusion protein comprising two moieties, wherein:
i) The first moiety comprises an antigen-binding domain that binds to human CD8α or human CD8β;
ii) The second moiety comprises a mutant IL-2 polypeptide;
and wherein the second moiety is linked to the first moiety via a linker.

5. The fusion protein of claim 4 wherein said first moiety comprises (a) an antibody or antigen-binding fragment thereof comprising one or two heavy chain polypeptides and one or two light chain polypeptides; (b) a single chain antibody or single chain variable fragment (scFv); or (c) a VHH antibody.

6. The fusion protein of any one of claims 1 to 5 wherein the fusion protein activates CD8+ T cells with 10-fold or greater potency, as compared to activation of NK cells.

7. The fusion protein of claim 6 wherein the fusion protein activates CD8+ T cells with 50-fold or greater potency, as compared to activation of NK cells.

8. The fusion protein of any one of claims 1-7 wherein said mutant IL-2 polypeptide exhibits reduced binding affinity by 50% or more to IL-2Rα polypeptide having an amino acid sequence of SEQ ID NO:2, compared to the binding affinity of the wild-type IL-2 polypeptide with an amino acid sequence of SEQ ID NO:1.

9. The fusion protein of claim 8 wherein said mutant IL-2 polypeptide exhibits reduced binding affinity by 50% or more to IL-2Rβ polypeptide having an amino acid sequence of SEQ ID NO:3, compared to the binding affinity of the wild-type IL-2 polypeptide with an amino acid sequence of SEQ ID NO:1.

10. The fusion protein of claim 8 or claim 9 wherein said mutant IL-2 polypeptide exhibits reduced binding affinity by 50% or more to IL-2Rγ polypeptide having an amino acid sequence of SEQ ID NO:4, compared to the binding affinity of the wild-type IL-2 polypeptide with an amino acid sequence of SEQ ID NO:1.

11. The fusion protein of any one of claims 1-10 wherein said mutant IL-2 polypeptide comprises the sequence of SEQ ID NO:1 with one or more or two or more amino acid substitutions relative to SEQ ID NO:1, and wherein the one or more or two or more substitution(s) comprise substitution(s) at positions of SEQ ID NO:1 selected from the group consisting of: Q11, H16, L18, L19, D20, Q22, R38, F42, K43, Y45, E62, P65, E68, V69, L72, D84, S87, N88, V91, 192, T123, Q126, S127, I129, and S130.

12. The fusion protein of claim 11, wherein the one or more or two or more substitution(s) comprise an F42A or F42K amino acid substitution relative to SEQ ID NO:1.

13. The fusion protein of claim 11 or claim 12, wherein the one or more or two or more substitution(s) further comprise an R38A, R38D, R38E, E62Q, E68A, E68Q, E68K, or E68R amino acid substitution relative to SEQ ID NO:1.

14. The fusion protein of any one of claims 11-13, wherein the one or more or two or more substitution(s) further comprise an H16E, H16D, D20N, M23A, M23R, M23K, S87K, S87A, D84L, D84N, D84V, D84H, D84Y, D84R, D84K, N88A, N88S, N88T, N88R, N88I, V91A, V91T, V91E, I92A, E95S, E95A, E95R, T123A, T123E, T123K, T123Q, Q126A, Q126S, Q126T, Q126E, S127A, S127E, S127K, or S127Q amino acid substitution relative to SEQ ID NO:1.

15. The fusion protein of any one of claims 11-14 wherein the one or more or two or more substitution(s) further comprise the amino acid mutation C125A compared to SEQ ID NO:1.

16. The fusion protein of any one of claims 1-10 wherein said mutant IL-2 polypeptide comprises an amino acid sequence selected from the group consisting of SEQ ID Nos:18-88.

17. The fusion protein of any one of claims 1-10 wherein said mutant IL-2 polypeptide comprises the amino acid sequence of SEQ ID NO:1 with one of the following sets of amino acid substitutions (relative to the sequence of SEQ ID NO:1): R38E and F42A; R38D and F42A; F42A and E62Q; R38A and F42K; R38E, F42A, and N88S; R38E, F42A, and N88A; R38E, F42A, and V91E; R38E, F42A, and D84H; H16D, R38E and F42A; H16E, R38E and F42A; R38E, F42A and Q126S; R38D, F42A and N88S; R38D, F42A and N88A; R38D, F42A and V91E; R38D, F42A, and D84H; H16D, R38D and F42A; H16E, R38D and F42A; R38D, F42A and Q126S; R38A, F42K, and N88S; R38A, F42K, and N88A; R38A, F42K, and V91E; R38A, F42K, and D84H; H16D, R38A, and F42K; H16E, R38A, and F42K; R38A, F42K, and Q126S; F42A, E62Q, and N88S; F42A, E62Q, and N88A; F42A, E62Q, and V91E; F42A, E62Q, and D84H; H16D, F42A, and E62Q; H16E, F42A, and E62Q; F42A, E62Q, and Q126S; R38E, F42A, and C125A; R38D, F42A, and C125A; F42A, E62Q, and C125A; R38A, F42K, and C125A; R38E, F42A, N88S, and C125A; R38E, F42A, N88A, and C125A; R38E, F42A, V91E, and C125A; R38E, F42A, D84H, and C125A; H16D, R38E, F42A, and C125A; H16E, R38E, F42A, and C125A; R38E, F42A, C125A and Q126S; R38D, F42A, N88S, and C125A; R38D, F42A, N88A, and C125A; R38D, F42A, V91E, and C125A; R38D, F42A, D84H, and C125A; H16D, R38D, F42A, and C125A; H16E, R38D, F42A, and C125A; R38D, F42A, C125A, and Q126S; R38A, F42K, N88S, and C125A; R38A, F42K, N88A, and C125A; R38A, F42K, V91E, and C125A; R38A, F42K, D84H, and C125A; H16D, R38A, F42K, and C125A; H16E, R38A, F42K, and C125A; R38A, F42K, C125A and Q126S; F42A, E62Q, N88S, and C125A; F42A, E62Q, N88A, and C125A; F42A, E62Q, V91E, and C125A; F42A, E62Q, and D84H, and C125A; H16D, F42A, and E62Q, and C125A; H16E, F42A, E62Q, and C125A; F42A, E62Q, C125A and Q126S; F42A, N88S, and C125A; F42A, N88A, and C125A; F42A, V91E, and C125A; F42A, D84H, and C125A; H16D, F42A, and C125A; H16E, F42A, and C125A; and F42A, C125A and Q126S.

18. The fusion protein of any one of claims 1-17 wherein the fusion protein binds human CD8, and wherein the binding of the fusion protein to CD8 does not block the interaction of CD8 with MEC class I.

19. The fusion protein of any one of claims 1-18 wherein said first and second Fc domains comprise the following Fc mutations according to EU numbering: L234A, L235A, G237A, and K322A.

20. The fusion protein of any one of claims 1-19 wherein:
(a) said first Fc domain comprises the following amino acid substitutions: Y349C and T366W, and wherein said second Fc domain comprises the following amino acid substitutions: S354C, T366S, L368A and Y407V, according to EU numbering; or
(b) said second Fc domain comprises the following amino acid substitutions: Y349C and T366W, and wherein said first Fc domain comprises the following amino acid substitutions: S354C, T366S, L368A and Y407V, according to EU numbering.

21. The fusion protein of any one of claims 1-20, wherein the fusion protein has one or more of the following properties:
(a) wherein the fusion protein binds human CD8, and wherein the binding of the fusion protein to CD8 does not block the interaction of CD8 with MEC class I; and
(b) activates CD8+ T cells with 10-fold or greater potency, as compared to activation of NK cells.

22. The fusion protein of any one of claims 6, 7, and 21, wherein potency of activation of CD8+ T cells and NK cells is measured by EC50 of cell activation, as assessed by cell proliferation.

23. One or more isolated polynucleotides encoding the mutant IL-2 polypeptides or fusion protein of any one of claims 1-22.

24. One or more vectors, particularly expression vectors, comprising the polynucleotides of claim claim 23.

25. A host cell comprising the polynucleotides of claim 23.

26. A pharmaceutical composition comprising the fusion protein according to any one of claims 1-22 and a pharmaceutically acceptable carrier.

27. The fusion protein of any one of claims 1-22 or composition of claim 26 for use as a medicament.

28. A method of treating cancer or chronic infection comprising administering an effective amount of the fusion protein according to any one of claims 1-22 or the composition of claim 26 to a patient.

29. A method of treating cancer comprising administering an effective amount of the fusion protein according to any one of claims 1-22 or the composition of claim 26 to a patient in combination with a T cell therapy, cancer vaccine, chemotherapeutic agent, or immune checkpoint inhibitor (ICI).

30. The method of claim 29, wherein the ICI is an inhibitor of PD-1, PD-L1, or CTLA-4.


SEQUENCE LISTING (88 sequences)

SEQ ID NO: 1 - protein, 133 aa, Artificial Sequence [sequence omitted]

SEQ ID NO: 2 - protein, 272 aa, Artificial Sequence
  Synthetic Construct
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 3 - protein, 551 aa, Artificial Sequence
  Synthetic Construct
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 4 - protein, 369 aa, Artificial Sequence
  Synthetic Construct
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 5 - protein, 5 aa, Artificial Sequence
  Synthetic Construct
  VARIANT 1, 2, 3, 4: Can be present in repeats of 1, 2, 3, 4, 5, 6, 7, 8, 9, 10, 11, or 12
  VARIANT 5: Can be present in repeats of 1, 2 or 3, or can be absent
GGGSG

SEQ ID NO: 6 - protein, 6 aa, Artificial Sequence
  Synthetic Construct
  VARIANT 1, 2, 3, 4, 5: Can be present in repeats of 1, 2, 3, 4, 5, 6, 7, 8, 9, 10, 11, or 12
  VARIANT 6: Can be present in repeats of 1, 2 or 3, or can be absent
GGGGSG

SEQ ID NO: 7 - protein, 7 aa, Artificial Sequence
  Synthetic Construct
  VARIANT 1, 2, 3, 4, 5, 6: Can be present in repeats of 1, 2, 3, 4, 5, 6, 7, 8, 9, 10, 11, or 12
  VARIANT 7: Can be present in repeats of 1, 2 or 3, or can be absent
GGGGGSG

SEQ ID NO: 8 - protein, 6 aa, Artificial Sequence
  Synthetic Construct
  VARIANT 1, 2, 3, 4, 5: Can be present in repeats of 2, 3, or 4
  VARIANT 6: Absent
GGGGSG

SEQ ID NO: 9 - protein, 16 aa, Artificial Sequence
  Synthetic Construct
  VARIANT 16: Absent
GGGGSGGGGSGGGGSG

SEQ ID NO: 10 - protein, 117 aa, Rattus norvegicus
EVQLVESGGGLVQPGRSLKLSCAASGFTFSNYYMAWVRQAPTKGLEWVAYINTGGGTTYYRDSVKGRFTISRDDAKSTLYLQMDSLRSEDTATYYCTTAIGYYFDYWGQGVMVTVSS

SEQ ID NO: 11 - protein, 107 aa, Rattus norvegicus
DIQLTQSPASLSASLGETVSIECLASEDIYSYLAWYQQKPGKSPQVLIYAANRLQDGVPSRFSGSGSGTQYSLKISGMQPEDEGDYFCLQGSKFPYTFGAGTKLELK

SEQ ID NO: 12 - protein, 117 aa, Rattus norvegicus
EVKLQESGPSLVQPSQTLSLTCSVSGFSLISDSVHWVRQPPGKGLEWMGGIWADGSTDYNSALKSRLSISRDTSKSQGFLKMNSLQTDDTAIYFCTSNRESYYFDYWGQGTMVTVSS

SEQ ID NO: 13 - protein, 106 aa, Rattus norvegicus
DIQMTQSPASLSASLGDKVTITCQASQNIDKYIAWYQQKPGKAPRQLIHYTSTLVSGTPSRFSGSGSGRDYSFSISSVESEDIASYYCLQYDTLYTFGAGTKLELK

SEQ ID NO: 14 - protein, 117 aa, Artificial Sequence
  Synthetic Construct
EVKLQESGPSLVQPSQTLSLTCSVSGFSLISDSVHWVRQPPGKGLEWMGGIWADGSTDYNSALKSRLSISRDTSKSQGFLKMNSLQTDDTAIYFCTSARESYYFDYWGQGTMVTVSS

SEQ ID NO: 15 - protein, 106 aa, Artificial Sequence
  Synthetic Construct
DIQMTQSPASLSASLGDKVTITCQASQNIDKYIAWYQQKPGKAPRQLIHYTSTLVSGTPSRFSGSGSGRDYSFSISSVESEDIASYYCLQYATLYTFGAGTKLELK

SEQ ID NO: 16 - protein, 121 aa, Mus musculus
EVQLVESGGALVQPGRSLKLSCAASGLTFSDCYMAWVRQTPTKGLEWVSYISSDGGSTYYGDSVKGRFTISRDNAKSTLYLQMNSLRSEDMATYYCACATDLSSYWSFDFWGPGTMVTVSS

SEQ ID NO: 17 - protein, 107 aa, Mus musculus
DIQMTQSPSSLPVSLGERVTISCRASQGISNNLNWYQQKPDGTIKPLIYHTSNLQSGVPSRFSGSGSGTDYSLTISSLEPEDFAMYYCQQDATFPLTFGSGTKLEIK

SEQ ID NO: 18 - protein, 133 aa, Artificial Sequence
  Synthetic Construct
APTSSSTKKTQLQLEHLLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 19 - protein, 133 aa, Artificial Sequence
  Synthetic Construct
APTSSSTKKTQLQLEHLLLDLQMILNGINNYKNPKLTDMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 20 - protein, 133 aa, Artificial Sequence
  Synthetic Construct
APTSSSTKKTQLQLEHLLLDLQMILNGINNYKNPKLTRMLTAKFYMPKKATELKHLQCLEEQLKPLEEVLNLAQSKNFHLRPRDLISNINVIVLELKGSETTFMCEYADETATIVEFLNRWITFCQSIISTLT

SEQ ID NO: 21 - protein, 133 aa, Artificial Sequence
  Synthetic Construct
APTSSSTKKTQLQLEHLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 22 - protein, 133 aa, Artificial Sequence
  Synthetic Construct
APTSSSTKKTQLQLEHLLLDLQMILNGINNYKNPKLTEMLTAKFYMPKKATELKHLQCLEEELKPLEEVLNLAQSKNFHLRPRDLISSINVIVLELKGSETTFMCEYADETATIVEFLNRWITFCQSIISTLT

SEQ ID NO: 23 - protein, 133 aa, Artificial Sequence
  Synthetic Construct
APTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFCQSIISTLT

SEQ ID NO: 24 - protein, 133 aa, Artificial Sequence
  Synthetic Construct
APTSSSTKKTQLQLEHLLLDLQMILNGINNYKNPKLTEMLTAKFYMPKKATELKHLQCLEEELKPLEEVLNLAQSKNFHLRPRDLISNINEIVLELKGSETTFMCEYADETATIVEFLNRWITFCQSIISTLT

SEQ ID NO: 25 - protein, 133 aa, Artificial Sequence
  Synthetic Construct
APTSSSTKKTQLQLEHLLLDLQMILNGINNYKNPKLTEMLTAKFYMPKKATELKHLQCLEEELKPLEEVLNLAQSKNFHLRPRHLISNINVIVLELKGSETTFMCEYADETATIVEFLNRWITFCQSIISTLT

SEQ ID NO: 26 - protein, 133 aa, Artificial Sequence
  Synthetic Construct
APTSSSTKKTQLQLEDLLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 27 - protein, 133 aa, Artificial Sequence
  Synthetic Construct
APTSSSTKKTQLQLEELLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 28 - protein, 133 aa, Artificial Sequence
  Synthetic Construct
APTSSSTKKTQLQLEHLLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFCSSIISTLT

SEQ ID NO: 29 - protein, 133 aa, Artificial Sequence
  Synthetic Construct
APTSSSTKKTQLQLEHLLLDLQMILNGINNYKNPKLTDMLTAKFYMPKKATELKHLQCLEEELKPLEEVLNLAQSKNFHLRPRDLISSINVIVLELKGSETTFMCEYADETATIVEFLNRWITFCQSIISTLT

SEQ ID NO: 30 - protein, 133 aa, Artificial Sequence
  Synthetic Construct
APTSSSTKKTQLQLEHLLLDLQMILNGINNYKNPKLTDMLTAKFYMPKKATELKHLQCLEEELKPLEEVLNLAQSKNFHLRPRDLISAINVIVLELKGSETTFMCEYADETATIVEFLNRWITFCQSIISTLT

SEQ ID NO: 31 - protein, 133 aa, Artificial Sequence
  Synthetic Construct
APTSSSTKKTQLQLEHLLLDLQMILNGINNYKNPKLTDMLTAKFYMPKKATELKHLQCLEEELKPLEEVLNLAQSKNFHLRPRDLISNINEIVLELKGSETTFMCEYADETATIVEFLNRWITFCQSIISTLT

SEQ ID NO: 32 - protein, 133 aa, Artificial Sequence
  Synthetic Construct
APTSSSTKKTQLQLEHLLLDLQMILNGINNYKNPKLTDMLTAKFYMPKKATELKHLQCLEEELKPLEEVLNLAQSKNFHLRPRHLISNINVIVLELKGSETTFMCEYADETATIVEFLNRWITFCQSIISTLT

SEQ ID NO: 33 - protein, 133 aa, Artificial Sequence
  Synthetic Construct
APTSSSTKKTQLQLEDLLLDLQMILNGINNYKNPKLTDMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 34 - protein, 133 aa, Artificial Sequence
  Synthetic Construct
APTSSSTKKTQLQLEELLLDLQMILNGINNYKNPKLTDMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 35 - protein, 133 aa, Artificial Sequence
  Synthetic Construct
APTSSSTKKTQLQLEHLLLDLQMILNGINNYKNPKLTDMLTAKFYMPKKATELKHLQCLEEELKPLEEVLNLAQSKNFHLRPRDLISNINVIVLELKGSETTFMCEYADETATIVEFLNRWITFCSSIISTLT

SEQ ID NO: 36 - protein, 133 aa, Artificial Sequence
  Synthetic Construct
APTSSSTKKTQLQLEHLLLDLQMILNGINNYKNPKLTAMLTKKFYMPKKATELKHLQCLEEELKPLEEVLNLAQSKNFHLRPRDLISSINVIVLELKGSETTFMCEYADETATIVEFLNRWITFCQSIISTLT

SEQ ID NO: 37 - protein, 133 aa, Artificial Sequence
  Synthetic Construct
APTSSSTKKTQLQLEHLLLDLQMILNGINNYKNPKLTAMLTKKFYMPKKATELKHLQCLEEELKPLEEVLNLAQSKNFHLRPRDLISAINVIVLELKGSETTFMCEYADETATIVEFLNRWITFCQSIISTLT

SEQ ID NO: 38 - protein, 133 aa, Artificial Sequence
  Synthetic Construct
APTSSSTKKTQLQLEHLLLDLQMILNGINNYKNPKLTAMLTKKFYMPKKATELKHLQCLEEELKPLEEVLNLAQSKNFHLRPRDLISNINEIVLELKGSETTFMCEYADETATIVEFLNRWITFCQSIISTLT

SEQ ID NO: 39 - protein, 133 aa, Artificial Sequence
  Synthetic Construct
APTSSSTKKTQLQLEHLLLDLQMILNGINNYKNPKLTAMLTKKFYMPKKATELKHLQCLEEELKPLEEVLNLAQSKNFHLRPRHLISNINVIVLELKGSETTFMCEYADETATIVEFLNRWITFCQSIISTLT

SEQ ID NO: 40 - protein, 133 aa, Artificial Sequence
  Synthetic Construct
APTSSSTKKTQLQLEDLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 41 - protein, 133 aa, Artificial Sequence
  Synthetic Construct
APTSSSTKKTQLQLEELLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 42 - protein, 133 aa, Artificial Sequence
  Synthetic Construct
APTSSSTKKTQLQLEHLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFCSSIISTLT

SEQ ID NO: 43 - protein, 133 aa, Artificial Sequence
  Synthetic Construct
APTSSSTKKTQLQLEHLLLDLQMILNGINNYKNPKLTRMLTAKFYMPKKATELKHLQCLEEQLKPLEEVLNLAQSKNFHLRPRDLISSINVIVLELKGSETTFMCEYADETATIVEFLNRWITFCQSIISTLT

SEQ ID NO: 44 - protein, 133 aa, Artificial Sequence
  Synthetic Construct
APTSSSTKKTQLQLEHLLLDLQMILNGINNYKNPKLTRMLTAKFYMPKKATELKHLQCLEEQLKPLEEVLNLAQSKNFHLRPRDLISAINVIVLELKGSETTFMCEYADETATIVEFLNRWITFCQSIISTLT

SEQ ID NO: 45 - protein, 133 aa, Artificial Sequence
  Synthetic Construct
APTSSSTKKTQLQLEHLLLDLQMILNGINNYKNPKLTRMLTAKFYMPKKATELKHLQCLEEQLKPLEEVLNLAQSKNFHLRPRDLISNINEIVLELKGSETTFMCEYADETATIVEFLNRWITFCQSIISTLT

SEQ ID NO: 46 - protein, 133 aa, Artificial Sequence
  Synthetic Construct
APTSSSTKKTQLQLEHLLLDLQMILNGINNYKNPKLTRMLTAKFYMPKKATELKHLQCLEEQLKPLEEVLNLAQSKNFHLRPRHLISNINVIVLELKGSETTFMCEYADETATIVEFLNRWITFCQSIISTLT

SEQ ID NO: 47 - protein, 133 aa, Artificial Sequence
  Synthetic Construct
APTSSSTKKTQLQLEDLLLDLQMILNGINNYKNPKLTRMLTAKFYMPKKATELKHLQCLEEQLKPLEEVLNLAQSKNFHLRPRDLISNINVIVLELKGSETTFMCEYADETATIVEFLNRWITFCQSIISTLT

SEQ ID NO: 48 - protein, 133 aa, Artificial Sequence
  Synthetic Construct
APTSSSTKKTQLQLEELLLDLQMILNGINNYKNPKLTRMLTAKFYMPKKATELKHLQCLEEQLKPLEEVLNLAQSKNFHLRPRDLISNINVIVLELKGSETTFMCEYADETATIVEFLNRWITFCQSIISTLT

SEQ ID NO: 49 - protein, 133 aa, Artificial Sequence
  Synthetic Construct
APTSSSTKKTQLQLEHLLLDLQMILNGINNYKNPKLTRMLTAKFYMPKKATELKHLQCLEEQLKPLEEVLNLAQSKNFHLRPRDLISNINVIVLELKGSETTFMCEYADETATIVEFLNRWITFCSSIISTLT

SEQ ID NO: 50 - protein, 133 aa, Artificial Sequence
  Synthetic Construct
APTSSSTKKTQLQLEHLLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 51 - protein, 133 aa, Artificial Sequence
  Synthetic Construct
APTSSSTKKTQLQLEHLLLDLQMILNGINNYKNPKLTDMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 52 - protein, 133 aa, Artificial Sequence
  Synthetic Construct
APTSSSTKKTQLQLEHLLLDLQMILNGINNYKNPKLTRMLTAKFYMPKKATELKHLQCLEEQLKPLEEVLNLAQSKNFHLRPRDLISNINVIVLELKGSETTFMCEYADETATIVEFLNRWITFAQSIISTLT

SEQ ID NO: 53 - protein, 133 aa, Artificial Sequence
  Synthetic Construct
APTSSSTKKTQLQLEHLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 54 - protein, 133 aa, Artificial Sequence
  Synthetic Construct
APTSSSTKKTQLQLEHLLLDLQMILNGINNYKNPKLTEMLTAKFYMPKKATELKHLQCLEEELKPLEEVLNLAQSKNFHLRPRDLISSINVIVLELKGSETTFMCEYADETATIVEFLNRWITFAQSIISTLT

SEQ ID NO: 55 - protein, 133 aa, Artificial Sequence
  Synthetic Construct
APTSSSTKKTQLQLEHLLLDLQMILNGINNYKNPKLTEMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 56 - protein, 133 aa, Artificial Sequence
  Synthetic Construct
APTSSSTKKTQLQLEHLLLDLQMILNGINNYKNPKLTEMLTAKFYMPKKATELKHLQCLEEELKPLEEVLNLAQSKNFHLRPRDLISNINEIVLELKGSETTFMCEYADETATIVEFLNRWITFAQSIISTLT

SEQ ID NO: 57 - protein, 133 aa, Artificial Sequence
  Synthetic Construct
APTSSSTKKTQLQLEHLLLDLQMILNGINNYKNPKLTEMLTAKFYMPKKATELKHLQCLEEELKPLEEVLNLAQSKNFHLRPRHLISNINVIVLELKGSETTFMCEYADETATIVEFLNRWITFAQSIISTLT

SEQ ID NO: 58 - protein, 133 aa, Artificial Sequence
  Synthetic Construct
APTSSSTKKTQLQLEDLLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 59 - protein, 133 aa, Artificial Sequence
  Synthetic Construct
APTSSSTKKTQLQLEELLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 60 - protein, 133 aa, Artificial Sequence
  Synthetic Construct
APTSSSTKKTQLQLEHLLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFASSIISTLT

SEQ ID NO: 61 - protein, 133 aa, Artificial Sequence
  Synthetic Construct
APTSSSTKKTQLQLEHLLLDLQMILNGINNYKNPKLTDMLTAKFYMPKKATELKHLQCLEEELKPLEEVLNLAQSKNFHLRPRDLISSINVIVLELKGSETTFMCEYADETATIVEFLNRWITFAQSIISTLT

SEQ ID NO: 62 - protein, 133 aa, Artificial Sequence
  Synthetic Construct
APTSSSTKKTQLQLEHLLLDLQMILNGINNYKNPKLTDMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 63 - protein, 133 aa, Artificial Sequence
  Synthetic Construct
APTSSSTKKTQLQLEHLLLDLQMILNGINNYKNPKLTDMLTAKFYMPKKATELKHLQCLEEELKPLEEVLNLAQSKNFHLRPRDLISNINEIVLELKGSETTFMCEYADETATIVEFLNRWITFAQSIISTLT

SEQ ID NO: 64 - protein, 133 aa, Artificial Sequence
  Synthetic Construct
APTSSSTKKTQLQLEHLLLDLQMILNGINNYKNPKLTDMLTAKFYMPKKATELKHLQCLEEELKPLEEVLNLAQSKNFHLRPRHLISNINVIVLELKGSETTFMCEYADETATIVEFLNRWITFAQSIISTLT

SEQ ID NO: 65 - protein, 133 aa, Artificial Sequence
  Synthetic Construct
APTSSSTKKTQLQLEDLLLDLQMILNGINNYKNPKLTDMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 66 - protein, 133 aa, Artificial Sequence
  Synthetic Construct
APTSSSTKKTQLQLEELLLDLQMILNGINNYKNPKLTDMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 67 - protein, 133 aa, Artificial Sequence
  Synthetic Construct
APTSSSTKKTQLQLEHLLLDLQMILNGINNYKNPKLTDMLTAKFYMPKKATELKHLQCLEEELKPLEEVLNLAQSKNFHLRPRDLISNINVIVLELKGSETTFMCEYADETATIVEFLNRWITFASSIISTLT

SEQ ID NO: 68 - protein, 133 aa, Artificial Sequence
  Synthetic Construct
APTSSSTKKTQLQLEHLLLDLQMILNGINNYKNPKLTAMLTKKFYMPKKATELKHLQCLEEELKPLEEVLNLAQSKNFHLRPRDLISSINVIVLELKGSETTFMCEYADETATIVEFLNRWITFAQSIISTLT

SEQ ID NO: 69 - protein, 133 aa, Artificial Sequence
  Synthetic Construct
APTSSSTKKTQLQLEHLLLDLQMILNGINNYKNPKLTAMLTKKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 70 - protein, 133 aa, Artificial Sequence
  Synthetic Construct
APTSSSTKKTQLQLEHLLLDLQMILNGINNYKNPKLTAMLTKKFYMPKKATELKHLQCLEEELKPLEEVLNLAQSKNFHLRPRDLISNINEIVLELKGSETTFMCEYADETATIVEFLNRWITFAQSIISTLT

SEQ ID NO: 71 - protein, 133 aa, Artificial Sequence
  Synthetic Construct
APTSSSTKKTQLQLEHLLLDLQMILNGINNYKNPKLTAMLTKKFYMPKKATELKHLQCLEEELKPLEEVLNLAQSKNFHLRPRHLISNINVIVLELKGSETTFMCEYADETATIVEFLNRWITFAQSIISTLT

SEQ ID NO: 72 - protein, 133 aa, Artificial Sequence
  Synthetic Construct
APTSSSTKKTQLQLEDLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 73 - protein, 133 aa, Artificial Sequence
  Synthetic Construct
APTSSSTKKTQLQLEELLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 74 - protein, 133 aa, Artificial Sequence
  Synthetic Construct
APTSSSTKKTQLQLEHLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFASSIISTLT

SEQ ID NO: 75 - protein, 133 aa, Artificial Sequence
  Synthetic Construct
APTSSSTKKTQLQLEHLLLDLQMILNGINNYKNPKLTRMLTAKFYMPKKATELKHLQCLEEQLKPLEEVLNLAQSKNFHLRPRDLISSINVIVLELKGSETTFMCEYADETATIVEFLNRWITFAQSIISTLT

SEQ ID NO: 76 - protein, 133 aa, Artificial Sequence
  Synthetic Construct
APTSSSTKKTQLQLEHLLLDLQMILNGINNYKNPKLTRMLTAKFYMPKKATELKHLQCLEEQLKPLEEVLNLAQSKNFHLRPRDLISAINVIVLELKGSETTFMCEYADETATIVEFLNRWITFAQSIISTLT

SEQ ID NO: 77 - protein, 133 aa, Artificial Sequence
  Synthetic Construct
APTSSSTKKTQLQLEHLLLDLQMILNGINNYKNPKLTRMLTAKFYMPKKATELKHLQCLEEQLKPLEEVLNLAQSKNFHLRPRDLISNINEIVLELKGSETTFMCEYADETATIVEFLNRWITFAQSIISTLT

SEQ ID NO: 78 - protein, 133 aa, Artificial Sequence
  Synthetic Construct
APTSSSTKKTQLQLEHLLLDLQMILNGINNYKNPKLTRMLTAKFYMPKKATELKHLQCLEEQLKPLEEVLNLAQSKNFHLRPRHLISNINVIVLELKGSETTFMCEYADETATIVEFLNRWITFAQSIISTLT

SEQ ID NO: 79 - protein, 133 aa, Artificial Sequence
  Synthetic Construct
APTSSSTKKTQLQLEDLLLDLQMILNGINNYKNPKLTRMLTAKFYMPKKATELKHLQCLEEQLKPLEEVLNLAQSKNFHLRPRDLISNINVIVLELKGSETTFMCEYADETATIVEFLNRWITFAQSIISTLT

SEQ ID NO: 80 - protein, 133 aa, Artificial Sequence
  Synthetic Construct
APTSSSTKKTQLQLEELLLDLQMILNGINNYKNPKLTRMLTAKFYMPKKATELKHLQCLEEQLKPLEEVLNLAQSKNFHLRPRDLISNINVIVLELKGSETTFMCEYADETATIVEFLNRWITFAQSIISTLT

SEQ ID NO: 81 - protein, 133 aa, Artificial Sequence
  Synthetic Construct
APTSSSTKKTQLQLEHLLLDLQMILNGINNYKNPKLTRMLTAKFYMPKKATELKHLQCLEEQLKPLEEVLNLAQSKNFHLRPRDLISNINVIVLELKGSETTFMCEYADETATIVEFLNRWITFASSIISTLT

SEQ ID NO: 82 - protein, 133 aa, Artificial Sequence
  Synthetic Construct
APTSSSTKKTQLQLEHLLLDLQMILNGINNYKNPKLTRMLTAKFYMPKKATELKHLQCLEEELKPLEEVLNLAQSKNFHLRPRDLISSINVIVLELKGSETTFMCEYADETATIVEFLNRWITFAQSIISTLT

SEQ ID NO: 83 - protein, 133 aa, Artificial Sequence
  Synthetic Construct
APTSSSTKKTQLQLEHLLLDLQMILNGINNYKNPKLTRMLTAKFYMPKKATELKHLQCLEEELKPLEEVLNLAQSKNFHLRPRDLISAINVIVLELKGSETTFMCEYADETATIVEFLNRWITFAQSIISTLT

SEQ ID NO: 84 - protein, 133 aa, Artificial Sequence
  Synthetic Construct
APTSSSTKKTQLQLEHLLLDLQMILNGINNYKNPKLTRMLTAKFYMPKKATELKHLQCLEEELKPLEEVLNLAQSKNFHLRPRDLISNINEIVLELKGSETTFMCEYADETATIVEFLNRWITFAQSIISTLT

SEQ ID NO: 85 - protein, 133 aa, Artificial Sequence
  Synthetic Construct
APTSSSTKKTQLQLEHLLLDLQMILNGINNYKNPKLTRMLTAKFYMPKKATELKHLQCLEEELKPLEEVLNLAQSKNFHLRPRHLISNINVIVLELKGSETTFMCEYADETATIVEFLNRWITFAQSIISTLT

SEQ ID NO: 86 - protein, 133 aa, Artificial Sequence
  Synthetic Construct
APTSSSTKKTQLQLEDLLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 87 - protein, 133 aa, Artificial Sequence
  Synthetic Construct
APTSSSTKKTQLQLEELLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 88 - protein, 133 aa, Artificial Sequence
  Synthetic Construct
APTSSSTKKTQLQLEHLLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFASSIISTLT